(19) United States
(12) Patent Application Publication
(10) Pub. No.: US 2004/0009165 A1
(43) Pub. Date: Jan. 15, 2004

(54) Lysosomal enzymes and lysosomal enzyme activators

(75) Inventors: Jens Sigurd Okkels, Vedbaek (DK); Anne Dam Jensen, Copenhagen NV (DK); Torben Halkier, Solroed Strand (DK); Rikke Bolding Jensen, Skibby (DK); Hans Thalsgard Schambye, Frederiksberg (DK)

Correspondence Address: MAXYGEN, INC., INTELLECTUAL PROPERTY DEPARTMENT, 515 GALVESTON DRIVE, RED WOOD CITY, CA, 94063, US

(73) Assignee: Maxygen ApS

(21) Appl. No.: 10/330,697

(22) Filed: Dec. 27, 2002

Related U.S. Application Data
(63) Continuation of application No. 09/753,126, filed on Dec. 29, 2000, now abandoned.
(60) Provisional application No. 60/217,497, filed on Jul. 11, 2000.
(60) Provisional application No. 60/211,124, filed on Jun. 12, 2000.
(60) Provisional application No. 60/210,984, filed on Jun. 12, 2000.
(60) Provisional application No. 60/174,652, filed on Jan. 6, 2000.

(30) Foreign Application Priority Data
Jun. 30, 2000 (DK) PA 2000 01027
Jun. 2, 2000 (DK) PA 2000 00865
Jun. 2, 2000 (DK) PA 2000 00866
Dec. 30, 1999 (DK) PA 1999 01891

(51) Int. Cl. A61K 38/46; C12N 9/36

(52) U.S. Cl. 424/94.62; 435/206

(57) ABSTRACT

A polypeptide selected from the group of lysosomal enzymes and lysosomal enzyme activators, comprising at least one introduced glycosylation site as compared to a corresponding parent enzyme or activator. By introducing additional glycosylation sites the resulting glycosylated lysosomal enzyme or activator obtains improved in vivo activity and thereby provides for improved treatment of lysosomal storage diseases.

### CROSS-REFERENCES TO RELATED APPLICATIONS

This application claims the benefit of and priority to Danish Patent Application PA 1999 01891 filed Dec. 30, 1999, U.S. Provisional Application No. 60/174,652 filed Jan. 6, 2000, Danish Application PA 200 00865 filed Jun. 2, 2000, U.S. Provisional Application No. 60/210,984 filed Jun. 12, 2000, U.S. Provisional Application No. 60/211,124 filed Jun. 12, 2000, Danish Application PA 2000 01027 filed Jun. 30, 2000, and U.S. Provisional Application No. 60/217,497 filed Jul. 11, 2000, the disclosures of which are incorporated herein by reference in the entirety for all purposes.

### FIELD OF INVENTION

The present invention relates to modified lysosomal enzymes and modified lysosomal enzyme activators having improved properties, methods of preparing such polypeptides and their use in therapy, in particular enzyme replacement therapy for the treatment of lysosomal storage diseases.

### BACKGROUND OF THE INVENTION

Lysosomes are acidic cytoplasmic organelles present in all animal cells. Lysosomes contain a variety of hydrolytic enzymes (lysosomal enzymes) that degrade internalized and endogenous macromolecular substrates such as sphingolipids present in the lysosymes. Deficiency of one or more of such enzymes leads to accumulation of undegraded substrate and eventually onset of a lysosomal storage disease. More than thirty distinct, inherited lysosomal storage diseases have been reported, some of which can be treated by presently available enzyme replacement therapy. Such diseases (and related lysosomal enzymes) include Fabry's disease (α-galactosidase), Farber's disease (ceramidase), Gaucher disease (glucocerebrosidase), Gₘₗ gangliosidosis (β-galactosidase), Tay-Sachs disease (β-hexosaminidase), Niemann-Pick disease (sphingomyelinase), Shindler disease (α-N-acetylgalactosaminidase), Hunter syndrome (iduronate-2-sulfatase), Sly syndrome (β-glucuronidase), Hurler and Huler/Scheie syndromes (iduronidase), I-Cell/San Filipo syndrome (mannose 6-phosphate transporter), Pombe's disease (α-glucosidase). The diseases and related enzymes are described in a variety of publications, see e.g. Scriver et al., The metabolic and molecular bases of inherited disease, volume II part 12, Lysosomal enzymes, pp. 2427-2882, New York McGraw-Hill 1995, and U.S. Pat. No. 5,929,304. For instance, U.S. Pat. No. 5,580,757 discloses expression of alpha-galactosidase.

Activators of lysosomal enzymes are known, examples of which are the Saposins. Saposin A (SapA), Saposin B (SapB), Saposin C (SapC) and Saposin D (SapD) are generated in lysosomes from a common precursor, called prosaposin, whose proteolytic cleavage begins in the late endosomes ((Nakano et al., J. Biochem. (Tokyo) 105, 152-154, 1989; Gavrieli-Rorman and Grabowski, Genomics 5, 486-492, 1989), Vielhaber et al. J. Biol. Chem. 271, 32438-32446, 1996). All Saposins appear to be involved in the lysosomal degradation of sphingolipids. A patient lacking all four saposins showed a combined sphingolipid storage disorder. So far selective deficiences of saposins are only known for SapB and SapC. Mutations affecting the coding region of SapB cause a variant form of metachromatic leukodistrophy with storage of sulfatides (Schlote et al., Eur. J. Pediatr. 150, 584-591, 1991). This, together with in vitro data, suggests SapB to be an activator of arylsulfatase A in vivo. SapC is a small 80 amino acid peptide which is an essential co-factor for the in vivo activity of GCB (Qi et al., J. Biol. Chem. 271, 6874-6880, 1996). SapC has been proposed to bind to GCB in vivo and introduce a conformational change in the enzyme thereby maximizing its catalytic activity (Grace et al., 1994 J. Biol. Chem; 269; 2283-2291; Qi & Grabowski, 1998, Biochemistry 37; 11544-11554). So far the actual physiological function of SapA and D has not been firmly established, but a role for SapA in the degradation of glucosylceramide and galactosylceramide has been hypothesized and mice studies have indicated its role in activation of galactocerebrosidase (Oral information, VIII International Congress of Inborn Errors of Metabolism, Cambridge, UK, Sep. 13-17, 2000). SapD have been suggested to be involved in the ceramide hydrolysis (Vaccaro et al. Neurochemical Research, 24, 307-314, 1999). Mice studies have indicated that SapD may be an in vivo activator of α-galactosidase (Oral information, VIII International Congress of Inborn Errors of Metabolism, Cambridge, UK, Sep. 13-17, 2000).

Gaucher's disease is an autosomal recessive disease resulting in a deficiency of the lysosomal hydrolase, acid β-glucosidase also termed glucocerebrosidase (E.C. 3.2.1.45) or GCB hereinafter. Gaucher's disease has been classified in three subtypes, cf. the table below.

| Clinical Features | Type I | Type II | Type III |
|---|---|---|---|
| Clinical Onset | Childhood/Adulthood | Infancy | Childhood |
| Hepatosplenomegaly | + | + | + |
| Hematologic | + | + | + |
| Complications | | | |
| Skeletal Involvement | + | − | + |
| Neurologic Involvement | − | + | + |
| Survival | Variable | <2 yrs | 2^{nd}-4^{th} |
| | | | decade |
| Ethnic predilection | Ashkenazic Jewish | Panethnic | Nothern |
| | | | Swedish |

There is a wide variability in the pattern and severity of disease involvement between and within each subtype. All three variants of Gaucher's disease are inherited “storage” diseases but are distinguished by the presence or absence of neurologic complications. The defect causes progressive accumulation of undegraded glycolipid substrates, particularly glucosylceramide, in reticuloendothelial cells and results in infiltration of the bone marrow, hepatosplenomegaly, and skeletal complications. Gaucher's disease is the most common inheritable lysosomal disease and occurs with a frequency of 1/40000-1/60000 in Caucasians and 1/1000 in Ashkenazi Jews.

The only existing treatment is enzyme substitution that has become available in the last decade. Initially, enzyme purified from human placentas (Ceredase™) was used, but patients are currently being switched to recombinantly produced enzyme, termed Cerezyme™. The enzyme is dispensed intraveneously (IV) up to three times a week. The treatment appears to be effective in removing many of the symptoms as well as correcting the paraclinical abnormalities except the neurological symptoms seen in type 2 and 3.

GCB is necessary for the breakdown of a particular fatty substance, glucosylceramide, to glucose and ceramide, by hydrolysis of the O-β-D-glucosidic linkage. It has been shown that the in vitro activity of the protein is elevated by the presence of acidic lipids, such as phosphatidylserine, and SapC. The enzyme is a lysosomal membrane protein but although the enzyme has substantial hydrophobic properties, no evidence for a transmembrane segment has been found. It has been shown by fluorescence spectroscopy, that the protein binds lipids and enters the membrane to some degree (Qi & Grabowski, 1998, Biochemistry 37; 11544-11554). It has been suggested that the role of SapC is to bind to GCB and introduce a conformational change in the enzyme thereby maximizing the catalytic activity (Qi & Grabowski, 1998, Biochemistry 37; 11544-11554).

The gene encoding human GCB was first sequenced in 1985 (Sorge et al., 1985, Proc. Natl. Acad Sci.; 2; 7289-7293). The protein consists of 497 amino acids derived from a 536-mer pro-peptide. The enzyme contains 4 glycosylation sites and 22 lysines. The recombinantly produced enzyme (Cerezyme™) differs from the placental enzyme (Ceredase™) in position 495 where an arginine has been substituted with a histidine. Furthermore, the oligosaccharide composition differs between the recombinant and the placental GCB as the former has more fucose and N-acetyl-glucosamine residues while the latter retains one high mannose chain. Both types of GCBs are treated with three different glycosidases (neuraminidase, galactosidase, and β-N acetyl-glucosaminidase) to expose terminal mannoses, which enables targeting of phagocytic cells. A pharmaceutical preparation comprising the recombinantly produced enzyme is described in U.S. Pat. No. 5,549,892.

WO 89/05850 discloses a clone of GCB and its expression in invertebrate cells.

WO 90/07573 discloses a recombinant enzymatically active GCB produced by a eukaryotic cell such as an insect, yeast or mammalian cell. The enzyme comprises as least one exposed mannose residue for binding to the mannose receptor of phagocytic cells.

EP 401 362 B1 discloses the production of GCB in CHO cells. The GCB is indicated to include an oligosaccharide moiety with at least one exposed mannose residue and preferably 2-4 mannose residues.

U.S. Pat. No. 5,433,946 discloses lectin-lysosomal enzyme conjugates and their use in treatment of lysosomal storage diseases. Glucocererbrosidase is mentioned as one enzyme among many to be modified and used in accordance with the teaching of U.S. Pat. No. 5,433,946.

U.S. Pat. No. 5,929,304 discloses production of lysosomal enzymes, exemplified by GCB, in transgenic plant cells.

U.S. Pat. No. 5,705,153 discloses GCB conjugates with non-antigenic polymers such as polyethylene glycol. The conjugates are claimed to exhibit enhanced turnover time and prolonged in vivo activity.

The drawbacks of the previously suggested forms of GCB have been an insufficient targeting of GCB to phagocytic cells. It has been shown that while 50-60% of administrated enzyme in mice was taken up by the liver, only approximately 10% was correctly targeted to liver phagocytic cells (Kupffer cells) (Bijsterbosch et al., 1996, Eur. J. Biochem, 237; 344-349 and Friedmann et al.,1999, Blood, 93; 2807-2816). This incorrect targeting, combined with a short half-life in serum (minutes) and in lysosomes (2-12 hours), results in a non-optimal treatment of Gaucher patients.

Doebber et al., J. Biol. Chem., 257, pp2193⁻²199, 1982 reports enhanced macrophage uptake of synthetically glycosylated human placental GCB.

One drawback associated with existing lysosomal enzyme replacement therapy treatment is that the in vivo bioactivity of the enzyme is undesirably low, e.g. because of low uptake and/or reduced targeting to lysosomes of the specific cells where the substrate is accumulated, and/or a short functional in vivo half-life in the lysosomes. Because of the low in vivo bioactivity frequent injections are required in current therapy. Accordingly, a need exists for providing lysosomal enzymes with improved in vivo activity.

### SUMMARY OF THE INVENTION

The object of the present invention is to improve the in vivo bioactivity of lysosomal enzymes and thereby provide an improved treatment of lysosomal storage diseases. This is achieved by providing modified lysosomal enzymes and/or modified lysosomal enzyme activators with improved properties, such as improved uptake in lysosomal cells and improved functional in vivo half-life.

In one aspect the invention relates to a polypeptide selected from the group of lysosomal enzymes and lysosomal enzyme activators, which polypeptide comprises at least one introduced glycosylation site as compared to a corresponding, preferably naturally-occurring, parent enzyme or activator. By introducing additional glycosylation sites increased and/or specific glycosylation may be achieved which is contemplated to lead to an improved uptake in the relevant cells or organelles and increased functional in vivo half-life (presumably as a consequence of reduced proteolytic degradation).

In another aspect the invention relates to a chimeric polypeptide comprising a lysosomal enzyme unit linked to at least one unit of an activator for said enzyme or a targeting polypeptide capable of targeting phagocytic cells. Thereby, the uptake and in vivo activity is improved as compared to the lysosomal enzyme in itself.

The invention also provides for a conjugated polypeptide, the polypeptide part of which is selected from the group of a lysosomal enzyme and a lysosomal enzyme activator and has at least one introduced and/or at least one removed attachment group for a macromolecular moiety as compared to a corresponding parent polypeptide, the polypeptide part being conjugated to at least one macromolecular moiety different from an oligosaccharide moiety. Of particular interest is a macromolecular moiety that is a polymer molecule such as PEG.

In still further aspects, the invention relates to a nucleotide sequence encoding a polypeptide of the invention, a vector and host cell comprising said nucleotide sequence, as well as a method of producing the polypeptide.

In a further aspect, the invention relates to a method of improving at least one property of a lysosomal enzyme, such as increasing in vivo activity thereof, which method comprises introducing an additional glycosylation site (or attachment group for a non-oligosaccharide moiety) into the lysosomal enzyme, preferably at a position exposed at the surface of the protein, and producing the modified lysosomal enzyme under conditions ensuring that the enzymes is glycosylated (or conjugated to the non-oligosaccharide moiety).

In still further aspects, the invention relates to a pharmaceutical composition comprising a polypeptide of the invention and a pharmaceutically acceptable diluent, carrier or excipient and to the use of the polypeptide for the treatment or prevention of a lysosomal storage disease treatable by the polypeptide or for the manufacture of a medicament for treatment or prevention of such disease.

The general principle of the present invention is illustrated herein predominantly by modification of GCB and accordingly, a specific object is to provide enzymatically active forms of GCB with increased in vivo activity, in particular with increased targeting to phagocytic cells and/or increased lysosomal activity. However, it is generally believed that the concept described herein for modification of GCB is generally applicable to other lysosomal enzymes.

### BRIEF DESCRIPTION OF THE DRAWINGS

FIG. 1: Uptake (Dosis-respons) in J774E cells of selected GCB polypeptides compared to Cerezyme. Different concentrations (400 mU/ml-15 mU/ml) of the GCB polypeptides were incubated with the cells in absence (closed symbols) or in the presence of yeast mannan (open symbols) as described in Methods section. The amount of GCB polypeptide taken up by the cells was determined by GCB Activity Assay. A; Raw data. B; Data corrected for mannose baseline.

FIG. 2. Stability of selected GCB polypeptides in J774E cells compared to Cerezyme™. Briefly, cells were incubated with 40 mU/ml enzyme for 1 hr before washing the cells and then measuring the amount of enzyme left in the cells after 30 min, 1 hr, 2 hr, 3 hr, 4 hr, and 5 hr. using the GCB Activity Assay.

FIG. 3. Activation of GCB polypeptides and Cerezyme™ in response to increasing amount of phosphatidyl serine from Bovine brain using the assay described in Methods.

FIG. 4. Activation of GCB polypeptides and Cerezyme™ in response to increasing amounts of SapC. The assay was done at pH 4.7 and in the presence of 5 μg/ml phosphatidyl serine and increasing amounts of SapC. For details, see Methods. A; Raw data curves and B; normalized curves.

FIG. 5: A schematic drawing showing the principle of random introduction of glycosylation sites (as further described in Example 2).

FIG. 6: SDS-PAGE of PEGylated wtGCB. Mark 12™ is a Mw marker, available from Novex, San Diego, Calif. 5×, 20× and 120×, respectively, indicates a 5, 20 and 120 times molar excess of PEG relative to the number of lysine residues.

FIG. 7: Uptake in J774E cells of PEGylated wt GCB.

FIG. 8: Preferred oligosaccharide structures

### DETAILED DISCLOSURE OF THE INVENTION

Definitions

In the present context, the term “polypeptide” is intended to indicate any structural form (e.g. the primary, secondary or tertiary structure) of an amino acid sequence comprising more than 5 amino acid residues. Thus, the term is intended to include the folded form of the polypeptide, otherwise termed “protein”. The term polypeptide is used herein about any polypeptide of the invention in any form, whether a chimeric polypeptide or a polypeptide comprising a peptide addition. The “GCB polypeptide” is a polypeptide exhibiting GCB activity, i.e. a polypeptide which is capable of degrading a glycolipid substrate, in particular 4-MU-glucopyranoside or p-nitrophenyl-glucopyranoside as described in the Methods section hereinafter. Typically the GCB polypeptide comprises more than 100 amino acid residues such as more than 300 amino acid residues, e.g. 100-500 amino acid residues. A “SapC polypeptide” is a polypeptide exhibiting SapC activity, i.e. capability of activating a GCB polypeptide, e.g. demonstrated by use of the SapC activation assay of GCB described in the Methods section herein. Analogously, a “SapA polypeptide” is a polypeptide exhibiting SapA activity, a “SapB polypeptide” is a polypeptide exhibiting SapB activity and a “SapD polypeptide” is a polypeptide exhibiting SapD activity, such activities being determined by methods known in the art. Furthermore, the “polypeptide” may be derivatized and thus be in the form of a “conjugated polypeptide” comprising a macromolecular moiety.

The term “conjugated polypeptide” is intended to indicate a heterogeneous (in the sense of composite) molecule formed by the covalent attachment of one or more polypeptide(s) to one or more macromolecular moieties such as polymer molecules or oligosaccharide moieties. The term covalent attachment means that the polypeptide and the macromolecular moiety are either directly covalently joined to one another, or else are indirectly covalently joined to one another through an intervening moiety or moieties, such as a bridge, spacer, or linkage moiety or moieties. Preferably, the conjugated polypeptide is soluble at relevant concentrations and conditions, i.e. soluble in physiological fluids such as blood. The term “non-conjugated polypeptide” may be used about the polypeptide part of the conjugate. A glycosylated polypeptide constitutes one example of a conjugated polypeptide as used herein. Another example is a PEGylated polypeptide.

The term “wildtype” or “wt” is used about any naturally-occurring lysosomal enzyme or lysosomal enzyme activator, either it be isolated from its natural source or produced recombinantly (in the latter case the wt polypeptide has the amino acid sequence of the corresponding polypeptide isolated from its natural source). Thus, the term is used about any naturally-occurring human or other (e.g. primate or murine) lysosomal enzyme or activator, including allelic or other naturally-occuring variants or functional fragments exhibiting the relevant lysosomal enzyme or activator activity, preferably at least 25% of the activity of the corresponding wt enzyme or activator.

In the case of GCB it is well known that numerous naturally-occurring GCBs exist which differ from each other in one or more amino acid residues and the term “wtGCB” is intended to mean any such naturally-occurring GCB. For instance, the wtGCB is an endogenous enzyme purified from human cells, in particular human placenta, or an enzyme produced recombinantly on the basis of a gene or cDNA sequence encoding such naturally-occurring GCB. Specific examples of “wtGCB” cDNA sequences (as defined in the present context) are those described by Sorge et al., Proc. Natl. Acad. Sci. USA 82, 7289-7293, 1985 and in U.S. Pat. No. 5,879,680, the amino acid sequences of which are comprised in SEQ ID NO 1.

The term “parent” is used about the starting polypeptide to be modified in accordance with the invention. The parent polypeptide may be a wt polypeptide or a variant or functional fragment thereof. Typically, a “variant” shows at least 80% sequence identity with an amino acid sequence encoding the relevant wt polypeptide, in particular at least 90% identity, such as at least 95% identity. For instance, a GCB polypeptide variant shows at least 80% sequence identity with the amino acid sequence shown in SEQ ID NO 1, in particular at least 90% identity, such as at least 95% identity with said sequence. The sequence identity is calculated from the most optimal alignment of the relevant sequences using a suitable program (e.g. CLUSTAL W). A “functional fragment” of a full-length wt or variant polypeptide is typically deleted in one or more amino acid residues of the N- and/or C-terminal end, while retaining the qualitative activity of the full-length polypeptide. For instance, a functional fragment of a full-length GCB polypeptide comprises, e.g. at least 100 amino acid residues, such as 250-490 amino acid residues, and has GCB activity, preferably at least 25% of the GCB activity of the corresponding full-length GCB polypeptide. A functional fragment of a lysosomal enzyme comprises at least the catalytic site of the enzyme.

The term “increased in vivo activity” is defined as 1) increased or prolonged activity in patients such that a lower dosage and/or less frequent infusions lead to equal or better treatment efficacy as compared to that obtained by the unmodified enzyme or by conventional GCB or other lysosomal enzyme therapy, 2) increased or prolonged activity in mononuclear cells, more preferably in the isolated lysosomes, harvested from patients treated with a polypeptide of the invention as compared to that obtained by a reference molecule, 3) increased or prolonged activity in phagocytic cells, e.g. Kupfer cells or peritoneal macrophages, isolated from mice pre-treated with a polypeptide of the invention as compared to that obtained by a reference molecule, 4) increased or prolonged activity in macrophage like cell lines, more preferably in isolated lysosomes therefrom, after exposure to a polypeptide of the invention (essentially as described below in the experimental section) as compared to that obtained by a reference molecule, 5) improved uptake of the polypeptide in the lysosomes of phagocytic cells, e.g. macrophage like cells, as compared to a reference molecule, 6) increased half-life of the polypeptide in the lysosomes as compared to that of a reference molecule, and/or 7) increased stability in serum and/or in phagocytic cells/lysosomes, e.g. seen as decreased sensitivity to proteolytic degradation, increased half-life and the like, as compared to a reference molecule.

The “reference molecule” is normally the parent polypeptide or an available commercial product comprising the parent polypeptide. For instance, in the case of a GCB polypeptide, the reference molecule is typically Cerezyme™ or Ceredase™ or a recombinantly produced wtGCB, e.g. the enzyme resulting from expression of the cDNA sequence shown in U.S. Pat. No. 5,879,680 in an sf9 insect cell (e.g. as described in Example 1 hereinafter).

Increased or prolonged activity as used above is conveniently measured in terms of increased functional in vivo half-life. The term “functional in vivo half-life” is used in its normal meaning, i.e. the time in which 50% of the enzyme activity of the polypeptide is retained under in vivo conditions, e.g. under the conditions mentioned above. Preferably, the term is applied to the enzyme activity in macrophage like cells isolated from patients or animals treated with the enzyme or in lysosomes isolated from these cells.

The term “increased” as used about the in vivo activity, or the serum or the functional in vivo half-life is used to indicate that the relevant activity or half-life of the polypeptide is statistically significant increased relative to that of a reference molecule. Preferably, the increased in vivo activity (i.e. any of the specific properties listed above or any combination of two or more of such properties) of a polypeptide of the invention is at least 110% of that of a reference molecule (e.g. the unmodified enzyme), in particular at least 120%, such as at least 130% or 140%, when measured under comparable conditions. Even more preferably, the increased in vivo activity is at least 150%, such as at least 160% or at least 170% or at least 200% of that of a reference molecule (e.g. the unmodified enzyme). For instance, the functional in vivo half-life is at least 10% higher, such as at least 50% higher, preferably at least 100% higher than that of a wt parent polypeptide, e.g. wtGCB.

The term “immunogenicity” as used in connection with a polypeptide of the invention is intended to indicate the ability of the polypeptide to induce a response from the immune system. The immune response may be a cell or antibody mediated response (see, e.g., Roitt: Essential Immunology (8^{th} Edition, Blackwell) for further definition of immunogenicity). Normally, reduced antibody reactivity will be an indication of reduced immunogenicity.

The term “reducing the immunogenicity” is intended to indicate that the polypeptide of the invention gives rise to a measurably lower immune response than a reference molecule as determined under comparable conditions. The reduced immunogenicity may be determined by use of any suitable method known in the art, e.g. in vivo or in vitro.

The term “attachment group” is intended to indicate a functional group of an amino acid residue group capable of attaching a macromolecular moiety such as a polymer molecule, an oligosaccharide moiety, a lipophilic molecule or an organic derivatizing agent. Useful attachment groups and their matching macromolecular moieties are apparent from the table below.

| | | Examples of | Conjugation | |
|---|---|---|---|---|
| Attachment | | macromolecular | method/Activated | |
| group | Amino acid | moiety | PEG | Reference |
| —NH₂ | N-terminal, Lys | Polymer, e.g. PEG | mPEG-SPA | Shearwater Inc. |
| | | | Tresylated | Delgado et al, |
| | | | mPEG | critical reviews |
| | | | | in Therapeutic |
| | | | | Drug Carrier |
| | | | | Systems |
| | | | | 9(3,4): 249-304 |
| | | | | (1992) |
| —COOH | C-term, Asp, Glu | Polymer, e.g. PEG | mPEG-Hz | Shearwater Inc |
| | | (Oligosaccharide | (In vitro | |
| | | moiety) | glycosylation) | |
| —SH | Cys | Polymer, e.g. PEG, | PEG- | Shearwater Inc |
| | | | vinylsulphone | Delgado et al, |
| | | | PEG-maleimide | critical reviews |
| | | | | in Therapeutic |
| | | Oligosaccharide | | Drug Carrier |
| | | moiety | In vitro | Systems |
| | | | glycosylation | 9(3,4): 249-304 |
| | | | | (1992) |
| —OH | Ser, Thr, OH—, | Oligosaccharide | In vivo O-linked | |
| | Lys | moiety | glycosylation | |
| —CONH₂ | Asn as part of an | Oligosaccharide | In vivo N- | |
| | N-glycosylation | moiety | glycosylation | |
| | site | | | |
| | | Polymer, e.g. PEG | | |
| Aromatic | Phe, Tyr, Trp | Oligosaccharide | In vitro | |
| residue | | moiety | glycosylation | |
| —CONH₂ | Gln | Oligosaccharide | In Vitro | Yan and Wold, |
| | | moiety | glycosylation | Biochemistry, |
| | | | | 1984, Jul 31; |
| | | | | 23(16): 3759-65 |
| Guanidino | Arg | Oligosaccharide | In vitro | Lundblad and |
| | | moiety | glycosylation | Noyes, Chimical |
| | | | | Reagents for |
| | | | | Protein |
| | | | | Modification, |
| | | | | CRC Press Inc. |
| | | | | Boca Raton, FI |
| Imidazole ring | His | Oligosaccharide | In vitro | As for guanidine |
| | | moiety | glycosylation | |

For in vivo N-glycosylation, the term “attachment group” is used in an unconventional way to indicate the amino acid residues constituting an N-glycosylation site (with the sequence N-X′-S/T/C-X″, wherein X′ is any amino acid residue except proline, X″ any amino acid residue that may or may not be identical to X′ and preferably is different from proline, N is asparagine and S/T/C is either serine, threonine or cysteine, preferably serine or threonine, and most preferably threonine). Although the asparagine residue of the N-glycosylation site is the one to which the oligosaccharide moiety is attached during in vivo glycosylation, such attachment cannot be achieved unless the other amino acid residues of the N-glycosylation site is present. Accordingly, when the macromolecular moiety is an oligosaccharide moiety and the conjugation is to be achieved by N-glycosylation, the term “amino acid residue comprising an attachment group for the macromolecular moiety” as used in connection with alterations of the amino acid sequence of the parent GCB is to be understood as amino acid residues constituting an N-glycosylation site is/are to be altered in such a manner that either a functional N-glycosylation site is introduced into the amino acid sequence or removed from said sequence. Normally, the term “glycosylation site” is used herein about an attachment group for an oligosaccharide moiety.

The term “macromolecular moiety” (which may also be termed non-peptide moiety) is intended to indicate any molecule, different from a peptide polymer composed of amino acid monomers and linked together by peptide bonds, which molecule is capable of conjugating to an attachment group of the polypeptide of the invention. Examples of such molecule include oligosaccharides (attached by in vivo or in vitro glycosylation) and polymers (as further described in the section entitled “Conjugation to a non-oligosaccharide macromolecular moiety”. The term “polymer molecule” may be used interchangeably with “polymeric group”. Except where the number of macromolecular moieties, such as polymeric groups, in the conjugate is expressly indicated, every reference to a macromolecular moiety referred to herein is intended as a reference to one or more such moieties of the conjugate.

The term “introduce” used in relation to an amino acid residue comprising an attachment group for a macromolecular moiety, e.g. a glycosylation site, is primarily intended to mean substitution of one or more existing amino acid residues, but may also mean insertion or deletion of an additional amino acid residue. The term “remove” is primarily intended to mean substitution of the amino acid residue(s) to be removed with (an)other amino acid residue(s), but may also mean deletion (without substitution) of the amino acid residue to be removed.

In the present application, amino acid names and atom names (e.g. CA, CB, NZ, N, O, C, etc) are used as defined by the Protein DataBank (PDB) which are based on the IUPAC nomenclature (IUPAC Nomenclature and Symbolism for Amino Acids and Peptides (residue names, atom names e.t.c.), Eur. J. Biochem., 138, 9-37 (1984) together with their corrections in Eur. J. Biochem., 152, 1 (1985). The term “amino acid residue” is intended to indicate an amino acid residue contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The terminology used for identifying amino acid positions/substitutions is illustrated as follows: K7 (indicates position #7 occupied by a lysine residue in the amino acid sequence shown in SEQ ID NO 1). K7N (indicates that the lysine residue of position 7 has been replaced with an asparagine). The numbering of amino acid residues made herein is made relative to the amino acid sequence shown in SEQ ID NO 1. Multiple substitutions are indicated with a “+”, e.g. K7N+F9T means an amino acid sequence which comprises a substitution of the lysine residue in position 7 with an asparagine and a substitution of the phenylalanine residue in position 9 with a threonine residue.

The Polypeptide of the Invention

Introduction of Glycosylation Site(s)

One important modification of lysosomal enzymes and lysomal enzyme activators described herein is related to changing the glycosylation profile of the enzymes and activators, with respect to the number of attached oligosaccharide moieties, and/or the composition of the oligosaccharide moieties. In particular, the invention is focused on providing a modified lysosomal enzyme or lysosomal enzyme activator with an increased number of high-mannose oligosaccharide moieties as compared to the corresponding parent, e.g., wt enzyme or activator.

Conveniently, the glycosylation profile of the lysosomal enzyme or lysosomal enzyme activator is altered by introducing and/or removing glycosylation sites in the amino acid sequence of the enzyme or activator, and producing the modified enzyme or activator under conditions providing for the desired glycosylation. The glycosylation is described further below in the section entitled “Glycosylation”.

In a first aspect the polypeptide of the invention is selected from the group of lysosomal enzymes and lysosomal enzyme activators comprising at least one introduced glycosylation site as compared to a corresponding parent, preferably naturally-occurring, enzyme or activator. In other words, the polypeptide of the invention has an amino acid sequence that differs from that of a parent polypeptide in that it comprises at least one introduced glycosylation site.

i) Introduction of Glycosylation Site in Mature Sequence

In one embodiment the glycosylation site(s) is introduced into the amino acid sequence of the mature form of the parent lysosomal enzyme or activator. For instance, for modification of GCB the glycosylation site is introduced within the amino acid sequence shown in SEQ ID NO 1. For instance, for modification of SapC, the glycosylation site is introduced within the amino acid sequence shown in SEQ ID NO 3.

The type of glycosylation site to be introduced is selected so as to provide the desired glycosylation profile.

The glycosylation site may be an in vitro or in vivo glycosylation site. For instance, the in vitro glycosylation site is selected from the group consisting of the N-terminal amino acid residue of the polypeptide, the C-terminal residue of the polypeptide, lysine, cysteine, arginine, glutamine, aspartic acid, glutamic acid, serine, tyrosine, histidine, phenylalanine and tryptophan, i.e. any of the attachment groups apparent from the table above in the definitions section. Of particular interest is an in vitro glycosylation site that is an epsilon-amino group, in particular as part of a lysine residue. Preferably, the glycosylation site is an in vivo glycosylation site. The introduction of an in vivo glycosylation site is normally performed by insertion, deletion or substitution of one or more amino acid residues that are selected so that a functional N— or O-glycosylation site is introduced into the amino acid sequence. Preferably, the amino acid residue(s) are inserted or substituted so that the resulting glycosylation site is located on the surface of the protein. For instance, it is desirable that the N-residue of an N-glycosylation site or the S or T residue of an O-glycosylation site is located at the surface of the polypeptide. Since charged amino acids are normally located on the surface of the protein, at least one of the amino acid residues to be modified in order to introduce a glycosylation site is preferably a charged amino acid residue or an amino acid residue located between position −4 and +4 relative to a charged amino acid residue (i.e. up to four amino acid residues located towards the N-terminal of the polypeptide relative to the charged amino acid residue, or up to 4 amino acids located towards the C-terminal of the polypeptide relative to the charged amino acid residue). Such residue is preferably selected from the group consisting of E, D, R, K, and H, and is most preferably K. It is understood that one or more of the amino acid residues located between position −4 and +4 relative to a charged amino acid residue may be modified in order to generate an in vivo (N- or O-) glycosylation site or an in vitro glycosylation site.

Furthermore, in order to ensure efficient glycosylation it is preferred that the in vivo glycosylation site, in particular the N residue of the N-glycosylation site or the S or T residue of the O-glycosylation site, is located in the N-terminal part of the lysosomal enzyme or activator, preferably in the part which precedes (and thus is outside) the last 50 C-terminal residues of the polypeptide. Also of preference is to introduce the in vivo glycosylation site in a position wherein only one mutation is required to create the site (i.e. where any other amino acid residues required for creating a functional glycosylation site are already present in the polypeptide). Further considerations as to the choice of position for introduction of an additional glycosylation site include that the amino acid residue to be introduced is not conserved in amino acid sequences homologous to the wt lysosomal enzyme or activator and/or is not found in the relevant position of the mutated lysomal enzyme of any lysosomal storage disease patient.

In order to increase the likelihood of the polypeptide being O-glycosylated it may be advantageous to introduce appropriate O-glycosylation sites into the polypeptide sequence. The peptide signal sequence for protein O-glycosylation is not fully characterized, although an in vitro study proposed that the sequence motif, XTPXP, serves as a signal for mucin-type O-glycosylation. Asada et al. Glycoconj J 16(7):321-326, 1999 showed that the AATPAP sequence (SEQ ID NO:5) acts as an efficient O-glycosylation signal, in vivo in CHO-cells. In yeast cells O-glycosylation of serine and threonine residues have been reported in many cases but with no clear consensus sequence for O-glycosylation. In one case a serine residue was O-glycosylated by inserting eight amino acid residues (TGRGDSPA; SEQ ID NO:6) into lysozyme (Yamada et al., Biochemistry 33(13), 3885-3889, 1994). New introduced O-glycosylation sites may therefore also be chosen from these sequences. Furthermore, such sites can be constituted by serine and/or threonine rich regions, i.e. amino acid regions comprising at least two serine and/or threonine residues in a stretch of 10 amino acid residues, in particular at least three, four, five or six such residues in a stretch of 10 amino acid residues, or at least two such residues in a stretch of 8, 6 or 4 amino acid residues. The O-glycosylation site is preferably introduced by substitution of one or more amino acid residues located in position −5 to +5, such as −4 to +4 of any of the N-residues listed above in connection with introduction of N-glycosylation sites.

The in vivo glycosylation site is preferably an N-glycosylation site. N-glycosylation is a convenient way of achieving glycosylation, provides a desirable glycosylation profile when expressed in certain host cells, and is believed not to give rise to profound immunogenicity problems.

The polypeptide of the invention may comprise at least one introduced glycosylation site within the mature sequence, in particular 1-5 introduced glycosylation sites.

ii) Introduction of Glycosylation Site by Means of Peptide Addition

Furthermore, in addition to or as an alternative to introducing glycosylation site(s) within the amino acid sequence of the mature lysosomal enzyme or lysosomal enzyme activator, additional glycosylation site(s) may be introduced by means of a peptide addition. In this case the polypeptide comprises or consists or consists essentially of the primary structure,

NH₂—X—P—COOH or NH₂—P—X—COOH,

wherein

X is a peptide addition comprising or contributing to a glycosylation site, and P is the polypeptide to be modified, i.e. a lysosomal enzyme or activator thereof, e.g. a parent polypeptide as defined herein or a modified polypeptide having introduced and/or removed glycosylation sites in the mature part of the polypeptide.

In the context of a peptide addition the term “comprising a glycosylation site” is intended to mean that a complete glycosylation site is present in the peptide addition, whereas the term “contributing to a glycosylation site” is intended to cover the situation, wherein at least one amino acid residue of an N-glycosylation site is present in the peptide addition, whereas the other amino acid residue of said site is present in the polypeptide P, whereby the glycosylation site can be considered to bridge the peptide addition and the polypeptide.

Usually, the peptide addition is fused to the N-terminal or C-terminal end of the polypeptide P as reflected in the above shown structure so as to provide an N- or C-terminal elongation of the polypeptide P. However, it is also possible to insert the peptide addition within the amino acid sequence of the polypeptide P whereby the polypeptide comprises, consists or consists essentially of the primary structure NH₂—Pₓ—X—P_{y}—COOH wherein

Pₓ is an N-terminal part of the relevant polypeptide P,

P_{y} is a C-terminal part of said polypeptide P, and

X is a peptide addition comprising or contributing to a glycosylation site.

In order to minimize structural changes effected by the insertion of the peptide addition within the sequence of the polypeptide P, it is desirable that it be inserted in a non-structural part thereof. For instance, Pₓ is a non-structural N-terminal part of a mature polypeptide P, and P_{y} is a structural C-terminal part of said mature polypeptide, or Pₓ is a structural N-terminal part of a mature polypeptide P, and P_{y} is a non-structural C-terminal part of said mature polypeptide.

The term “non-structural part” is intended to indicate a part of either the C- or N-terminal end of the folded polypeptide (e.g. protein) that is outside the first structural element, such as an α-helix or a β-sheet structure. The non-structural part can easily be identified in a three-dimensional structure or model of the polypeptide. If no structure or model is available, a non-structural part typically comprises or consists of the first or last 1-20 amino acid residues, such as 1-10 amino acid residues of the amino acid sequence constituting the mature form of the polypeptide.

When the peptide addition comprises only few amino acid residues, e.g. 1-5 such as 1-3 amino acid residues, and in particular 1 amino acid residue, the peptide addition can be inserted into a loop structure of the polypeptide P and thereby elongate said loop.

In principle the peptide addition X can be any stretch of amino acid residues ranging from a single amino acid residue to a mature protein. Usually, the peptide addition X comprises 1-500 amino acid residues, such as 2-500, normally 2-50 or 3-50 amino acid residues, such as 3-20 amino acid residues. The length of the peptide addition to be used for modification of the polypeptide P is dependent of or determined on the basis of a number of factors including the type of polypeptide to be modified and the desired effect to be achieved by the modification. The peptide addition may be designed by a site-specific or random approach, e.g as out-lined in further detail in the “Other Methods of the. Invention” section below and as exemplified in the Examples section herein.

Typically, the peptide addition X comprises 1-20, such as 1-10 glycosylation sites. For instance, the peptide addition X comprises 1, 2, 3, 4, 5, 6, 7, 8, 9 or 10 glycosylation sites. It is well known that one frequently occurring consequence of modifying an amino acid sequence of, e.g., a human protein is that new epitopes are created by such modification.: Macromolecular moieties may be used to to shield any new epitopes created by the peptide addition, and therefore it is desirable that sufficient glycosylation sites (or attachment groups for any other desirable macromolecular moiety) are present to enable shielding of all epitopes introduced into the sequence. This is e.g. achieved when the peptide addition X comprises at least one glycosylation site within a stretch of 30 contiguous amino acid residues, such as at least one glycosylation sites within 20 amino acid residues or at least one attachment group within 10 amino acid residues, in particular 1-3 attachment groups within a stretch of 10 contiguous amino acid residues in the peptide addition X.

Thus, in one embodiment the peptide addition X comprises at least two glycosylation sites, wherein two of said amino acid residues are separated by at most 10 amino acid residues, none of which comprises the glycosylation site in question.

Preferably, the glycosylation site of the peptide addition is an in vivo glycosylation site, preferably an N-glycosylation site. Accordingly, the peptide addition X comprises at least one N-glycosylation site, typically at least two N-glycosylation sites. For instance, the peptide addition X has the structure X₁—N—X₂-T/S/C-Z, wherein X₁ is a peptide comprising at least one amino acid residue or is absent, X₂ is any amino acid residue different from P, and Z is absent or a peptide comprising at least one amino acid residue. For instance, X₁ is absent, X₂ is an amino acid residue selected from the group consisting of I, A, G, V and S (all relatively small amino acid residues), and Z comprises at least 1 amino acid residue. For instance, Z can be a peptide comprising 1-50 amino acid residues and, e.g., 1-10 glycosylation sites.

Alternatively, XI comprises at least one amino acid residue, e.g. 1-50 amino acid residues, X₂ is an amino acid residue selected from the group consisting of I, A, G, V and S, and Z is absent. For instance, X₁ comprises 1-10 glycosylation sites.

For instance, the peptide addition for use in the present invention can comprise a peptide sequence selected from the group consisting of INAT/S, GNIT/S, VNIT/S, SNIT/S, ASNIT/S (SEQ ID NO:7), NIT/S, SPINAT/S (SEQ ID NO:8), ASPINAT/S (SEQ ID NO:9), ANIT/SANIT/SANI (SEQ ID NO:10), ANIT/SGSNIT/SGSNIT/S (SEQ ID NO:11), ASNST/SNNGT/SLNAT/S (SEQ ID NO:12), ANHT/SNET/SNAT/S (SEQ ID NO:13), GSPINAT/S (SEQ ID NO:14), ASPINAT/SSPINAT/S (SEQ ID NO:15), ANNT/SNYT/SNWT/S (SEQ ID NO:16), ATNIT/SLNYT/SANT/ST (SEQ ID NO:17), AANST/SGNIT/SINGT/S (SEQ ID NO:18), AVNWT/SSNDT/SSNST/S (SEQ ID NO:19), GNAT/S, AVNWT/SSNDT/SSNST/S (SEQ ID NO:20), ANNT/SNYT/SNST/S (SEQ ID NO:21), and ANNTNYTNWT (SEQ ID NO:22), wherein T/S is either a T or an S residue, preferably a T residue.

The peptide addition can comprise one or more of these peptide sequences, i.e. at least two of said sequences either directly linked together or separated by one or more amino acid residues, or can contain two or more copies of any of these peptide sequence. It will be understood that the above specific sequences are given for illustrative purposes and thus do not constitute an exclusive list of peptide sequences of use in the present invention.

In a more specific embodiment the peptide addition X is selected from the group consisting of INAT/S, GNIT/S, VNIT/S, SNIT/S, ASNIT/S (SEQ ID NO:7), NIT/S, SPINAT/S (SEQ ID NO:8), ASPINAT/S (SEQ ID NO:9), ANIT/SANIT/SANI (SEQ ID NO:10), and ANIT/SGSNIT/SGSNIT/S (SEQ ID NO:11), wherein T/S is either a T or an S residue, preferably a T residue.

In one embodiment, the peptide addition X has an N residue in position −2 or −1, and the polypeptide P or Pₓ has a T or an S residue in position +1 or +2, respectively, the residue numbering being made relative to the N-terminal amino acid residue of P or Pₓ, whereby an N-glycosylation site is formed. For instance, the polypeptide has a T or S residue in position 2, preferably a T residue, and the peptide addition is AN or comprises AN as the C-terminal amino acid residues.

Removal of Glycosylation Site

In addition or as an alternative to introducing a glycosylation site it may be desirable to remove one or more glycosylation sites of the parent polypeptide, for instance if such glycosylation site is located at the catalytic site of a parent lysosomal enzyme and thus, when glycosylated, will lead to reduced or no enzymatic activity. Accordingly, the polypeptide of the invention may lack at least one glycosylation site present in the parent naturally-occurring enzyme or activator, typically a glycosylation site located in a functional site of the parent polypeptide such as a catalytic site of the lysosomal enzyme. The glycosylation site to be removed may be an in vivo or in vitro glycosylation site. When removing a glycosylation site this is preferably done by substitution, preferably to a conversative substitutions. Conservative substitution tables providing functionally similar amino acids are well known in the art. The table below sets forth six groups which contain amino acids that are “conservative substitutions” for one another.

| | | | | |
|---|---|---|---|---|
| 1 | Alanine (A) | Serine (S) | Threonine (T) | |
| 2 | Aspartic acid (D) | Glutamic acid (E) | | |
| 3 | Asparagine (N) | Glutamine (Q) | | |
| 4 | Arginine (R) | Lysine (K) | | |
| 5 | Isoleucine (I) | Leucine (L) | Methionine (M) | Valine (V) |
| 6 | Phenylalanine (F) | Tyrosine (Y) | Tryptophan (W) | |

Number of Glycosylation Sites

Irrespectively of how additional glycosylation sites are provided (whether in the mature part of the polypeptide or by means of a peptide addition), the polypeptide of the invention normally comprises 1, 2, 3, 4, 5, 6, 7, 8, 9, 10, 11, 12, 13, 14, 15 or more introduced glycosylation sites, in particular N-glycosylation sites, the upper limit being determined by the number of introduced glycosylation sites that can be introduced without substantially reducing the in vivo activity of the resulting polypeptide; Preferably, the polypeptide comprises 2-10 introduced glycosylation sites, e.g. at least 2-3 introduced glycosylation sites, such as 4-5 introduced glycosylation sites, in particular N-glycosylation sites. Analogously, 0-15 glycosylation sites may have been removed from the parent polypeptide, typically 0-5. The total number of glycosylation sites present in the polypeptide of the invention is normally in the range of 1-20, such as 3-15. For instance, the polypeptide of the inventon comprises 1, 2, 3, 4, 5, 6, 7, 8, 9, 10, 11, 12, 13, 14, 15 or more glycosylation sites.

Chimeric Polypeptides

In a further aspect the invention relates to a chimeric polypeptide comprising a lysosomal enzyme unit linked to one or more units of an activator of said enzyme. The term “unit” is intended to indicate a polypeptide having the activity of the enzyme or activator, respectively. For instance, a lysosomal enzyme unit comprises the amino acid sequence of the mature lysosomal enzyme, in case of GCB, e.g. the amino acid sequence of SEQ ID NO 1, optionally modified by one or more amino acid changes. Likewise, an activator unit comprises, e.g., the amino acid sequence of a mature activator, in the case of SapC, e.g. the amino acid sequence of SEQ ID NO 3, optionally modified by one or more amino acid changes.

The enzyme and/or activator constituents of the chimeric polypeptide may be any polypeptide exhibiting the relevant lysosomal enzyme or activator activity. For instance, the lysosomal enzyme constituent is a wt lysosomal enzyme or a variant or functional fragment thereof, or a modified lysosomal enzyme as described herein having introduced glycosylation site(s). Analogously, the activator may be a wt lysosomal enzyme activator or a variant or functional fragment thereof, or a modified activator as described herein having introduced glycosylation site(s).

While the enzyme and activator units may be linked by any type of linkage, in particular a covalent linkage, such as by chemical cross-linking using cross-linking agents known in the art, or by di-sulphide bridges, it is particularly preferred that the polypeptide constituents are linked via a peptide bond or a peptide linker (and thus that the chimeric polypeptide is a fusion polypeptide). If used, the linker peptide must be of a type (length, amino acid composition, amino acid sequence, etc) that is adequate to link the two (or more) polypeptide constituents in such a way that they assume a conformation relative to one another so that the resulting polypeptide has the relevant lysosomal enzyme activity. Furthermore, the linker peptide is typically designed to increase the stability of the polypeptide towards proteolytic degradation, e.g by use of special amino acid sequences or residues. The peptide linker sequence may comprise one or more glycosylation sites. For instance, the linker can contain the sequence NAT providing an N-glycosylation site.

The linker may, e.g., be 0-50 amino acid residues long. For instance, the linker peptide predominantly includes the amino acid residues Gly, Ser, Ala or Thr. A typical linker comprises 1-30 amino acid residues, such as a sequence of about 2-20 or 3-15 amino acid residues. The amino acid residues selected for inclusion in the linker peptide should exhibit properties that do not interfere significantly with the activity of the chimeric polypeptide. Thus, the linker peptide should on the whole not exhibit a charge which wouid be inconsistent with the lysosomal enzyme activity of the chimeric polypeptide, or interfere with internal folding, or form bonds or other interactions with amino acid residues in one or more of the polypeptide constituents which would seriously impede the binding of the chimeric polypeptide to the mannose receptor.

Specific linkers for use in the present invention may be designed on the basis of known naturally occurring as well as artificial polypeptide linkers (see, e.g., Hallewell et al. (1989), J. Biol. Chem. 264, 5260-5268; Alfthan et al. (1995), Protein Eng. 8, 725-731; Robinson & Sauer (1996), Biochemistry 35, 109-116; Khandekar et al. (1997), J. Biol. Chem. 272, 32190-32197; Fares et al. (1998), Endocrinology 139, 2459-2464; Smallshaw et al. (1999), Protein Eng. 12, 623-630; U.S. Pat. No. 5,856,456). For instance, linkers used for creating single-chain antibodies, e.g. a 15mer consisting of three repeats of a Gly-Gly-Gly-Gly-Ser: (SEQ ID NO:23) amino acid sequence ((Gly₄Ser)₃), are contemplated to be useful in the present invention. Furthermore, phage display technology as well as selective infective phage technology can be used to diversify and select appropriate linker sequences (Tang et al., J. Biol. Chem. 271, 15682-15686, 1996; Hennecke et al. (1998), Protein Eng. 11, 405-410). Also, the Arc repressor phage display has been used to optimise the linker length and composition for increased stability of the single-chain protein (Robinson and Sauer (1998), Proc. Natl. Acad. Sci. USA 95, 5929-5934).

Another way of obtaining a suitable linker is by optimizing a simple linker—e.g. ((Gly₄Ser)ₙ)—through random mutagenesis.

It will be clear from the present specification that whatever the nature of the linker, it should be one which is not readily susceptible to cleavage by e.g. proteases or chemical agents, since cleavage of the chimeric polypeptide to result in its polypeptide constituents is not desired in the present context.

In a further aspect the invention relates to a chimeric polypeptide comprising a lysosomal enzyme unit linked to one or more second polypeptide units, the second polypetide being capable of targeting phagocytic cells, preferably macrophages or macrophage like cells. The term “polypeptide targeting” is intended to indicate a polypeptide that is recognized and taken up by receptors present on phagocytic cells. Preferably, the lysosomal enzyme unit and the second polypeptide unit(s) are linked by a peptide bond or a peptide linker,

Examples of targeting polypeptides include the Fc region of immunoglobulins. Three classes of receptors for the Fc region of IgG have been identified in mice and humans (for a review see Fridman et al. Immunological Reviews 125, 49-76, 1992). The Fe receptor, FcγRI, bind monomeric IgG with high affinity and this receptor is found on monocytes, neutrophils and macrophages. The FcγR receptors mediate a large spectrum of functions. In macrophages they enable phagocytosis of IgG-coated particles, endocytosis of immune complexes to lysosomes (Ukkonen et al. J. Exp. Med. 163, 952-971, 1986) etc. A chimeric polypeptide comprising a lysosomal enzyme and the Fe part of IgG may therefore result in specific targeting of the chimeric polypeptide to macrophages by FcγR mediated endocytosis and may therefore be used in treatment of the relevant lysosomal storage disease, such as Gaucher's disease. Examples of chimeric polypeptides comprising Fc and a second polypeptide are described by Liu et al., Biochem. Biophys. Res. Comm. 197, 1094-1102, 1993, Dwyer et al., J. Biol. Chem. 274, 9738-9743, 1999 or Wang et al., Protein Engineering, 7, 715-722, 1994. Instead of a chimeric polypeptide either a monoclonal or polyclonal antibody against the lysosomal enzyme may be coadministered with the enzyme and result in Fc mediated uptake into macrophages.

Similarly may other receptors that are relative specific for macrophages be used for uptake of the lysosomal enzyme, such as GCB, by fusing the enzyme with the ligand for the receptor. Examples of such ligands are chemokines targeting a chemokine receptor specific for macrophages or lipoprotein targeting the scavenger receptor.

The chimeric polypeptide comprising the lysosomal enzyme and the second polypeptide may further comprise one or more units of an activator for the lysosomal enzyme in question.

The chimeric polypeptide of the invention may comprise more than one unit of the activator for the lysososomal enzyme and may comprise more than one type-of activator. Typically, the chimeric polypeptide comprises 1-5 units of the activator. The order of activator and lysosomal enzyme is not believed to be critical and thus the activator may be added N- and/or C-terminally to the lysosomal enzyme, or within a non-structural part thereof.

Specific Chimeric Polypeptides of the Invention

In a specific embodiment the Iysosomal enzyme unit of a chimeric polypeptide of the invention is a GCB polypeptide. Thus, for instance, the chimeric polypeptide comprises a GCB polypeptide and at least one unit of a targeting polypeptide and/or at least one unit of a GCB activator (i.e. a polypeptide that is capable of increasing the in vivo activity of the GCB polypeptide). For instance, the targeting polypeptide is Fc and/or the GCB activator is SapA or SapC, preferably SapC. The chimeric polypeptide can comprise, e.g. 1-5 GCB activator units, of which at least one is preferably SapC. For instance, the chimeric polypeptide comprises 1, 2, 3, or 4 units of SapC and 0, 1 or 2 units of SapA.

The activator may be located N-terminally or C-terminally to the GCB polypeptide. Specific examples of a chimeric polypeptide according to this embodiment are chimeric polypeptides comprising the following structure: GCB-SapA-SapC, SapA-GCB-SapC, SapC-GCB-SapA, SapC-GCB-SapC, wherein, preferably, the units are linked by a peptide bond or peptide linker as described elsewhere herein.

It will be understood that the chimeric polypeptids described in this section exhibits GCB activity, and when relevant further has the activity of SapC.

The GCB polypeptide unit may be a wtGCB or a functional-fragment or variant thereof as described herein. In particular, the GCB polypeptide may be a GCB polypeptide of the invention as described herein. For instance, a fragment of wildtype or mutant GCB can be used, which lacks at least one, e.g. 1-20, such as 1-10 amino acid residues at the C-terminus (when the GCB is positioned at the N-terminal part of the chimeric polypeptide and/or is linked to an activator in its C-terminal end) or N-terminus (when the GCB is positioned at the C-terminal part of the chimeric polypeptide or linked to an activator in its N-terminal end).

Other examples of chimeric polypeptides of the invention include a chimeric polypeptide comprising an Arylsulphatase A unit and at least one unit of Fc or SapB, e.g. 1-5 copies added at the N- and/or C-terminal of the lysosomal enzyme, and a chimeric polypeptide comprising an alpha-galactosidase unit and at least one unit of Fc or Sap B and/or SapD. e.g. 1-5 copies added at the N-and/or C-terminal of the alpha-galactosidase unit.

The Parent Polypeptide

The parent polypeptide to be modified in accordance with the general principle outlined above may be any lysosomal enzyme or lysosomal enzyme activator. Preferably, the lysosomal enzyme or activator is one that binds to a mannose receptor or a mannose-6-phosphate receptor. Examples of such lysosomal enzymes include of glucocerebrosidase (GCB), α-L-iduronidase, acid α-glucosidase, α-galactosidase, acid sphingomyelinase, galactocerebrosidase, arylsulphatase A, sialidase, and hexosaminidase. Examples of activators include SapA, SapB, SapC, SapD, and GM-2 activator (the latter activates hexosaminidase). These enzymes and activators are well-known in the art and the skilled person will be aware of how to clone the genes encoding these enzyme for use in modification according to the present invention.

A GCB Polypeptide of the Invention

In a preferred embodiment the lysosomal enzyme to be modified is a GCB polypeptide, and thus the polypeptide of the invention is a GCB polypeptide.

The present application is believed to be the first disclosure of a modified GCB polypeptide that has an amino acid sequence that differs from that of a wtGCB polypeptide by at least one amino acid residue, and has an increased in vivo activity relative to said wtGCB

In particular, the present application is believed to constitute the first disclosure of a GCB polypeptide comprising an amino acid sequence that differs from that of a parent GCB polypeptide in that at least one amino acid residue comprising an attachment group for a macromolecular moiety has been introduced or at least one amino acid residue comprising an attachment group for a macromolecular moiety has been removed, in order to render the polypeptide more susceptible to conjugation to such macromolecular moiety. The term “differs” as used in the present application is intended to allow for additional differences being present. Such GCB polypeptide is of particular interest for preparing a conjugated polypeptide, further comprising at least one covalently attached macromolecular moiety of a type capable of attaching to the introduced or removed amino acid residue.

Of particular interest is a GCB polypeptide comprising the modifications described above in the section entitled “introduction of glycosylation site(s)”. Accordingly, in one embodiment the GCB polypeptide is a glycosylated GCB polypeptide, which comprises at least one introduced glycosylation site as compared to a parent GCB polypeptide (whether it be in the mature part of the GCB polypeptide or as a peptide addition thereto).

In one embodiment, the parent GCB polypeptide to be modified according to the invention comprises or is constituted by an amino acid sequence that corresponds to that of a wtGCB, in particular the sequence shown in SEQ ID NO 1 in which the amino acid residue located in position 495 is either H or R, or a variant or functional fragment thereof. Thus, the GCB polypeptide of the invention may comprise or be a sequence of amino acids corresponding to the sequence of a wtGCB except for the modification(s) introduced into the sequence in accordance with the invention.

For convenience, the wtGCB having the amino acid sequence shown in SEQ ID NO 1 is used as the backbone for the modifications disclosed in the present section. However, it will be understood that other GCBs may constitute parent GCB polypeptides to be modified in accordance with the invention. Such parent polypeptides are conveniently modified in positions, which are equivalent to those identified in SEQ ID NO 1. An “equivalent position” is intended to indicate a position in the amino acid sequence of a given GCB, which is homologous (i.e. corresponding in position in either primary or tertiary structure) to a position in the amino acid sequence shown in SEQ ID NO 1. The “equivalent position” is conveniently determined on the basis of an alignment of members of the GCB sequence family, e.g. using the program CLUSTALW version 1.74 using default parameters (Thompson et al., 1994, CLUSTAL W: improving the sensitivity of progressive multiple sequence alignment through sequence weighting, position-specific gap penalties and weight matrix choice, Nucleic Acids Research, 22:4673-4680) or from published alignments. For instance, O'Neill et al., PNAS 86, 5049-5053, 1989 discloses an alignment of human and murine GCB genes.

When the attachment group to be introduced is a glycosylation site the modified GCB of the invention can be produced with an increased glycosylation as compared to that achievable through the four native N-glycosylation sites of wtGCB.

For instance, in order to introduce an N-glycosylation site into a parent GCB polypeptide of the invention the polypeptide comprises one or more substitutions, relative to the amino-acid sequence shown in SEQ ID NO: 1 or an equivalent position of another backbone, selected from the group consisting of K7N+F9T, K7N+*9T, K7N+*9S (*9T and *9S represent an insertion of a threonine and serine residue, respectively, between amino acid residues S8 and F9), K7N+F9S, K74N+Q76T, K74N+Q76S, K77N+K79T, K77N+K79S, K79N+F81T, K79N+F81S, K106N+Y108T, K106N+Y108S, K155N+K157T, K155N+K157S, K157N+P159T, K157N+P159S, K186N+N188T, K186N+N188S, K193N+S195T, K194N, K194T, K198N+Q200T, K198N+Q200S, K215N+L217T, K215N+L217S, E222N+K224T, K224N+Q226T, K224N+Q226S, K293N+L295T, K293N+L295S, K303N+V305T, K303N+V305S, K321N, K321N+T323S, K346N+W348T, K346N+W348S, K408N, K408N+T410S, K413N+P415T, K413N+P415S, K425N+1427T, K425N+1427S, K441N+D443T, K441N+D443S, K466N+V468T, K466N+V468S, K473N+P475T and K473N+P475S.

Additionally or alternatively, the polypeptide may comprise a substitution to an asparagine residue in one or more of the positions selected from the group consisting of P6, G10, Y11, C23, T36, Y40, T43, E50, A95, L105, Y108, M133, D137, P171, L175, W179, K194, H206, L240, A269, E235, F337, V343, E349, L354, Q362, S364, V398, H422, E429, V437, D453, R463, T482, G486, P28, L34, E41, T61, L66, A84, I130, T132, A136, S181, E152, P178, L185, H206, G255, A291, G250, V295, K321, G325, P332, I367, G377, D405, K408, P465, L480 and 1489 of the amino acid sequence shown in SEQ ID NO:1.

A preferred polypeptide of the invention comprises at least one of the following sets of mutations or any other specific mutations listed in Table 3 in Example 6 below.

K194N;

K224N+Q226T;

E41N;

E222N+k224T;

K303N+V305T;

E41N+K 194N+K224N+Q226T;

K194N+E222N+K224T+K303N+V305T;

E41N+K194N+K224N+Q226T+K303N+V305T;

D153N+K155T;

R163N+L165T;

T132N; and/or

I130N

Of the above mentioned specific mutants those are preferred which are outside the last 50 C-terminal amino acid residue of the parent GCB polypeptide and/or requires only one substitution to introduce an in vivo glycosylation site.

For instance, in order to introduce an in vitro glycosylation site into a parent GCB polypeptide, an amino acid residue constituting an in vitro glycosylation site, preferably a lysine residue, is introduced into one or more positions, relative to the amino acid sequence shown in SEQ ID NO: 1 or an equivalent position of another GCB backbone, selected from the group consisting of R2, R39, R44, R47, R48, R120, R131, R163, R170, R211, R257, R262, R277, R285, R339, R353, R359, R395, R433, R463, R495, R496, H60, H145, H162, H206, H223, H255, H273, H274, H290, H306, H311, H328, H365, H374, H419, H422, H451, H490, D24, D27, D87, D127, D137, D140, D141, D153, D203, D218, D258, D263, D282, D283, D298, D358, D380, D399, D405, D409, D443, D445, D453, D467, D474, E41, E50, E72, E111, E112, E151, E152, E222, E233, E235, E254, E300, E326, E340, E349, E388, E429, and E481. In vitro glycosylation sites other than lysine may be introduced in the same positions.

The GCB polypeptide of the invention having at least one introduced in vitro glycosylation site may have been further modified in that an in vitro glycosylation site present in the parent GCB polypeptide has been removed, e.g. to reduce the number of glycosylation sites to avoid too extensive glycosylation. For instance 1-5 such sites may be removed. The in vitro glycosylation site to be removed is e.g. located at a function site. In the present context the term “functional site” is intended to indicate one or more amino acid residues which is/are essential for or otherwise involved in the function or performance of GCB. Such amino acid residues are “located at” the functional site. The functional site may be determined by methods known in the art. Amino acid residues E340 and E235 of SEQ ID NO 2 have been found to be part of a functional site of wt human GCB, and any amino acid residue of the parts of SEQ ID NO 2 defined by amino acid residues 336-344 and 231-239 are contemplated to be located at a functional site.

For instance, when the in vitro glycosylation site is a lysine residue, a lysine residue present in the parent GCB can be substituted with another amino acid residue, preferably arginine, or deleted. For instance, at least one of the lysine residues located in a position selected from the group consisting of K7, K74, K77, K79, K106, K155, K157, K186, K193, K197, K215, K224, K293, K303, K321, K346, K408, K413, K425, K441, K466 and K473 of the amino acid sequence shown in SEQ ID NO:1 has been replaced with another amino acid residue, in particular a lysine residue, or deleted.

In yet another embodiment the GCB polypeptide of the invention has been modified so as to obtain reduced susceptibility to proteolytic degradation. It is presently contemplated that a proteolytic cleavage site is located around amino acid residue 136 of wtGCB. Accordingly, in one embodiment the A GCB polypeptide of the invention comprises a modification at any of amino acid residues 132-139 relative to SEQ ID NO 1, resulting in reduced susceptibility to proteolytic degradation. One convenient way of achieving shielding of a proteolytic site is by use of a macromolecular moiety, in particular a polymer or an oligosaccharide moiety. For this purpose, the GCB polypeptide according to this embodiment may be modified so as to have introduced an attachment group for said moiety (e.g. a glycosylation site) into an equivalent position of the parent GCB polypeptide relative to amino acid residues 132-139 of SEQ ID NO 1. For instance, an N-glycosylation site is introduced so that the N-residue of said site occupies any of positions 132-139. Alternatively, a proline is introduced into any such position. Specific mutations believed to provide reduced proteolytic cleavage include: A136N, A135P or A136P.

A modified SapC Polypeptide of the Invention

In another embodiment the lysosomal enzyme activator to be modified in accordance with the invention is SapC. In particular, the parent SapC polypeptide has the sequence shown in SEQ ID NO 3. While the parent SapC may be modified to introduce any attachment group for a macromolecular moiety, it is presently preferred that it be modified by introduction of a glycosylation site, in particular an in vivo glycosylation site such as an an N-glycosylation site. In this case the SapC polypeptide of the invention may comprise at least one mutation selected from the group consisting of S1N+V3T/S, D2N+Y4T/S, K13N+V15T/S, E14N, K17N+I19T/S, I19N+N21T/S, E25N+E27T/S, K26N+I28T/S, D30N+F32T/S, D33N+M35T/S, K38N+P40T/S, S42N, S44N+E46T/S, and V51N (relative to SEQ ID NO 3), wherein T/S indicates a threonine or a serine residue, preferably a threonine residue.

SapC has been expressed recombinantly in E. coli (Qi et al., J. Biol. Chem. 269, 16746-16753, 1994), but apparently not in glycosylating host cells. Accordingly, in a further aspect the invention relates to a recombinant glycosylated SapC polypeptide. The glycosylated SapC polypeptide may be wtSapC or a variant or functional fragment thereof of a modified SapC polypeptide as described in the present application.

Preferably, the SapC polypeptide of the invention has at least one of the following properties:

It enhances the in vivo activity of endogenous glucocerebrosidase activity,

It enhances the in vivo activity of glucocerebrosidase in a patient to which glucocerebrosidase has been administered,

It exhibits an increased uptake in phagocytic cells, preferably macrophages or macrophage like cells,

It exhibits increased activity or functional in vivo half-life in lysosomes or under conditions mimicking lysosomal conditions, and/or

It increases an in vitro bioactivity of glucocerebrosidase.

The Methods section comprises suitable assays for determing such activities.

The SapC polypeptide according to the invention finds particular use in therapy, alone or in combination with GCB (wtGCB or a commercially available GCB or a GCB polypeptide of the present invention), or as a constituent of a chimeric polypeptide of the invention.

Glycosylation

In most cases, the polypeptide of the invention is glycosylated (i.e. comprises an in vivo attached N- or O-linked oligosaccharide moiety or in vitro attached oligosaccharide moiety) and furthermore has an altered glycosylation profile as compared to that of the parent polypeptide. For instance, the altered glycosylation profile is a consequence of an altered, normally increased, number of attached oligosaccharide moieties and/or an altered type of attached oligosaccharide moieities.

The type of oligosaccharide moiety should normally be one that exhibits sufficient affinity for or uptake by a mannose receptor, thereby enabling the glycosylated polypeptide of the invention to exhibit improved affinity for or uptake by such receptor.

In the present context the term “mannose receptor” is intended to indicate any mannose receptor of interest in the present invention, including, in particular, a macrophage mannose receptor (of relevance for GCB) and a mannose-6-phosphate receptor (of relevance for some of the other lysosomal enzymes). Such improved affinity for or uptake by the mannose receptor is expected to result in increased uptake in phagocytic cells, preferably monocytes, macrophages (e.g: Kupffer cells, glia/mikroglia, alveolar phagocytes, reticulum cells, or other peripheral macrophages) or macrophage like cells (for instance osteoclasts, dendritic cells, or astrocytes). Also, increased lysosomal activity of the polypeptide is expected. Consequently, increased in vivo activity of the polypeptide and thereby increased therapeutic utility may result.

Furthermore, the type of oligosaccharide moiety to be attached should normally be one that does not lead to increased immunogenicity of the modified polypeptide as compared to that of the parent polypeptide, but rather equal or reduced immunogenicity as compared to the parent, in particular when the glycosylated lysosomal enzyme or activator is to be used in therapy.

The oligosaccharide moiety is preferably one provided by in vivo glycosylation. In order to achieve in vivo glycosylation of a polypeptide which has been modified by introduction of one or more glycosylation sites as described above, a nucleotide sequence encoding the polypeptide should be inserted in a glycosylating, eucaryotic expression host. The expression host cell may be selected from fungal (filamentous fungal or yeast), insect or animal cells or from transgenic plant cells. Also, the glycosylation may be achieved in the human body when using a nucleotide sequence encoding the polypeptide of the invention in gene therapy. Insect cell mediated in vivo N-glycosylation has proven to be of particular relevance for the present invention. Expression of the polypeptide in any of the above host cells may also result in the polypeptide being O-glycosylated at one or more serine or threonine residues.

It will be apparent from the description above that to obtain an improved uptake by the mannose receptor, at least one oligosaccharide chain of the glycosylated polypeptide of the invention comprises at least one exposed mannose residue. The term “mannose residue” is used generally about any functional mannose-based derivative, such as a mannosyl residue and a mannosyl phosphate group, capable of binding to a mannose receptor. The term “exposed” is intended to indicate that the oligosaccharide chain terminates with a mannose residue or that the mannose residue is located in such a position in the 3-D structure of the polypeptide, that it is readily available to bind with a mannose receptor protein. More preferably, when the polypeptide comprises more that one oligosaccharide chain, at least 50% of such chains, in particular at least 75% or all of such chains comprises at least 1 exposed mannose residue, in particular at least 2 exposed mannose residues, more preferably at least. 3 exposed mannose residues, e.g. 1-5 exposed mannose residues. For instance, at least one, such as two, three or all of the oligosaccharide chains comprises 2, 3, 4, 5 or 6 exposed mannose residues.

In addition to exposed mannose residues the oligosachharide chain(s) of the glycosylated polypeptide of the invention may comprise additional, non-exposed mannose residues. For instance, at least one of the oligosaccharide chains comprises 1-20 non-exposed mannose residues, such as 2-1 0 non-exposed mannose residues.

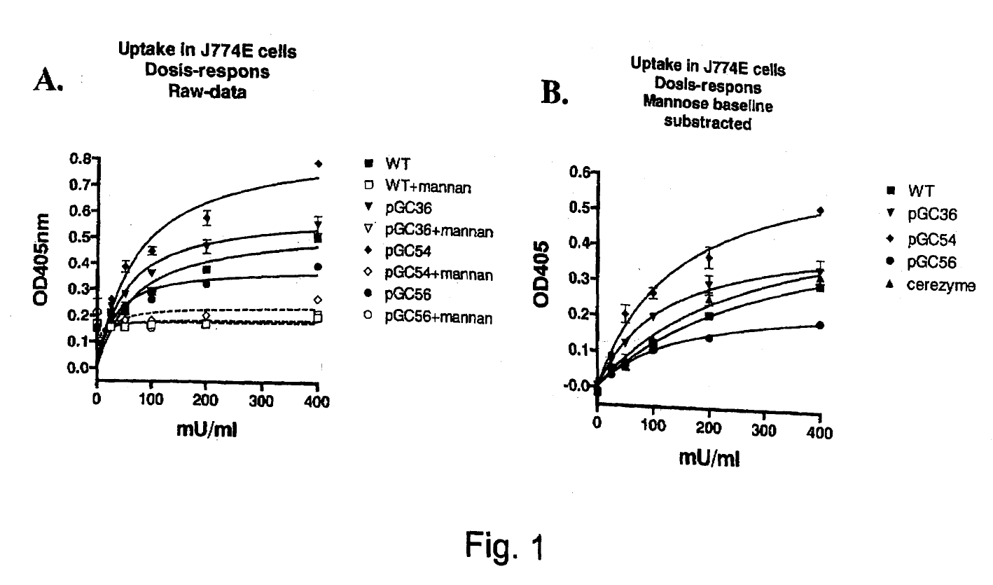

Examples of preferred oligosaccharide structures with exposed mannose residues are shown in FIG. 1 of U.S. Pat. No. 5,236,838, the contents of which are incorporated herein by reference, as well as in the Examples section herein.

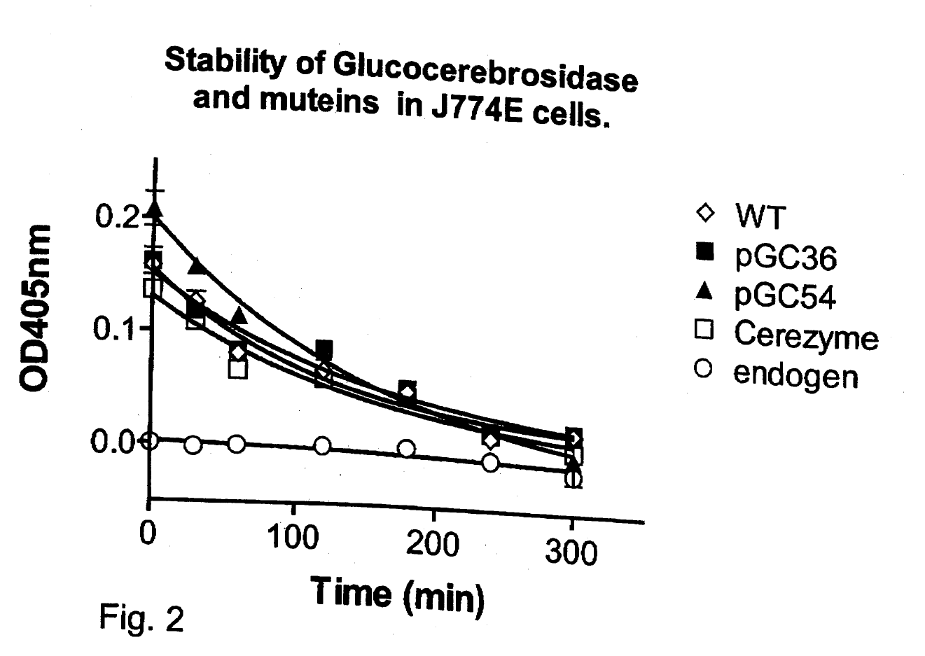

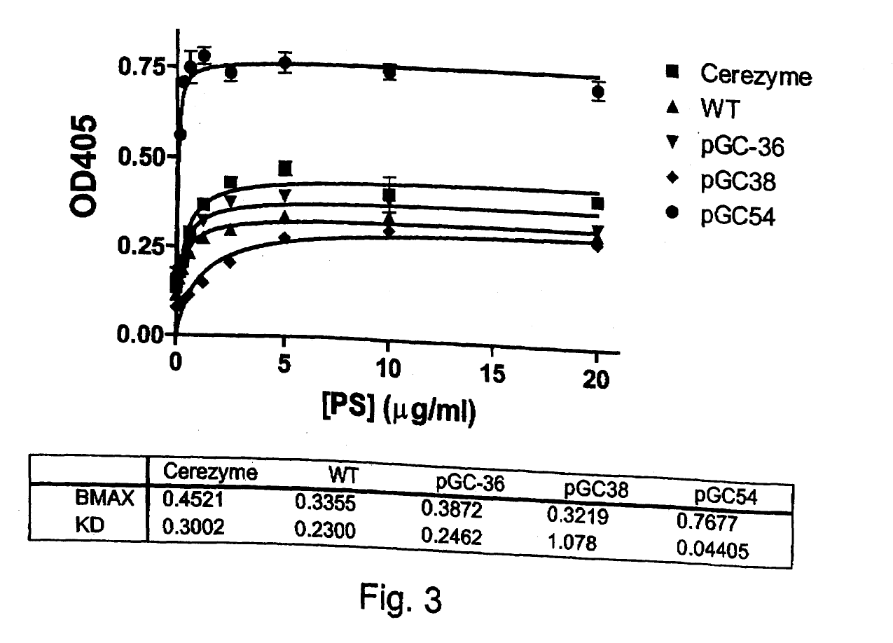

Expressed differently, the glycosylated polypeptide of the invention comprises at least one N-linked oligosaccharide chain being of the high mannose type (as defined in U.S. Pat. No. 5,218,092 or in FIG. 2 of Gemmill et al., Biochimica et Biophysica Acta 1426 (1999) 227-237, the contents of which are incorporated herein by reference). Expression in insect cells and in yeast cells has been found to provide glycosylated polypeptides with such oligosaccharides (see the examples herein). Furthermore, the polypeptide may comprise at least one O-linked oligosaccharide, e.g. having any of the structures disclosed in FIG. 3 of Gemmill et al., Biochimica et Biophysica Acta 1426 (1999) 227-237.

In one embodiment, in addition to mannose residues the glycosylated polypeptide of the invention may comprise at least one fucose residue. In another embodiment the glycosylated polypeptide is free of fucose, since, sometimes, fucose gives rise to immunogenicity. A fucose residue may be removed by subjecting the glycosylated polypeptide comprising such residue to treatment with a fucosidase and recovering the resulting fucose free glycosylated polypeptide.

In particular, a polypeptide of the invention comprises at least one oligosaccharide moiety with the following structure:

Asn-N-N-M-M₂

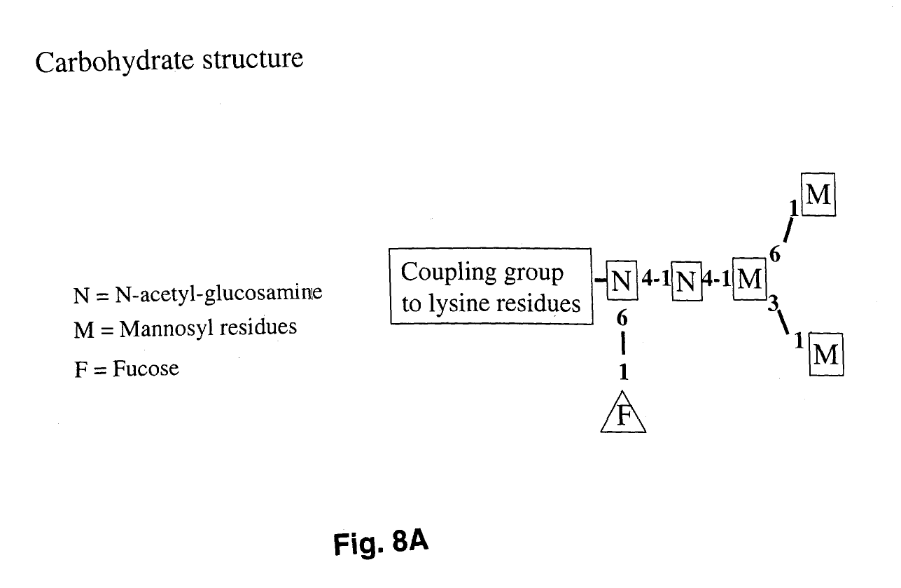

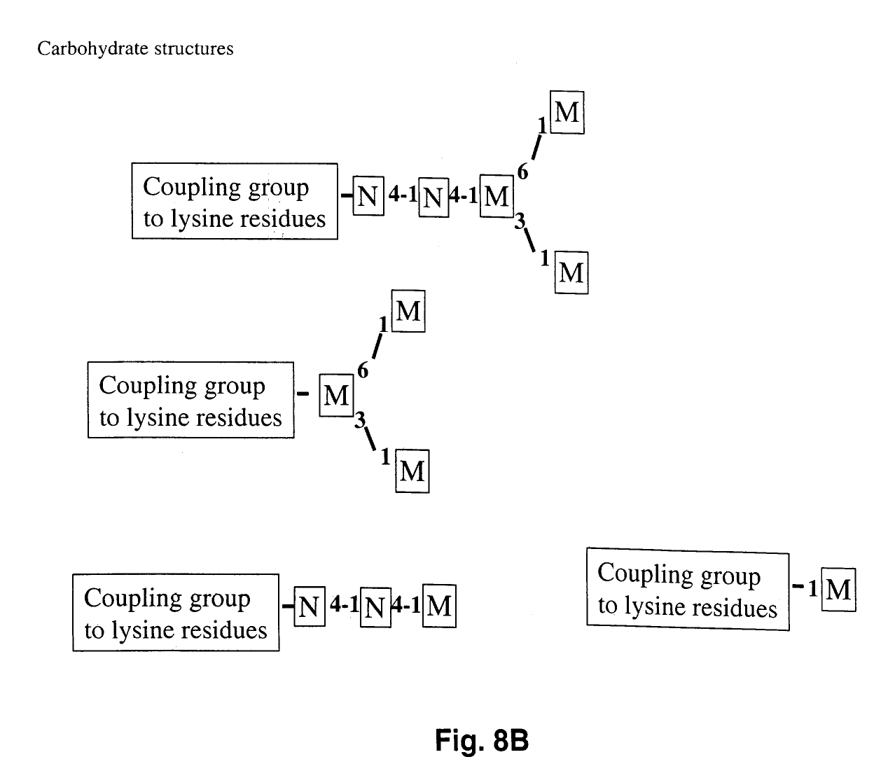

F

wherein Asn indicates the Asn residue of the polypeptide to which the oligosaccharide chain is attached, N an N-acetylglucosamine residue, F a fucose residue which may or may not be present and M-M₂ three mannose residues, two of which are linked to the same third mannose residue. Other preferred oligosaccharide structures are any of the oligosaccharides described in the Examples section hereinafter, or any of the structures shown in FIG. 8. Such structures may be provided by N-glycosylation or by in vitro glycosylation.

The nature and number of oligosaccharide moieties of a glycosylated polypeptide of the invention may be determined by a number of different methods known in the art e.g.by lectin binding studies (Reddy et al., 1985, Biochem. Med. 33: 200-210; Cummings, 1994, Meth. Enzymol. 230: 66-86; Protein Protocols (Walker ed.), 1998, chapter 9); by reagent array analysis method (RAAM) sequencing of released oligosaccharides (Edge et al., 1992, Proc. Natl. Acad. Sci. USA 89: 6338-6342; Prime et al., 1996, J. Chrom. A 720: 263-274); by RAAM sequencing of released oligosaccharides in combination with mass spectrometry (Klausen, et al., 1998, Molecular Biotechnology 9: 195-204); or by combining proteolytic degradation, glycopeptide purification by HPLC, exoglycosidase degradations and mass spectrometry (Krogh et al, 1997, Eur. J. Biochem. 244: 334-342). Specific methods for determining the glycosylation profile is described in the examples section hereinafter.

When the polypeptide is expressed in glycosylating host cells, which do not naturally provide exposed mannose residues (e.g. a mammalian cell), the glycosylated polypeptide of the invention is preferably subjected to enzymatic treatment subsequent to its expression to remove non-mannose sugar residues. The enzymatic treatment may, e.g., be as described in U.S. Pat. No. 5,549,892, the contents of which are incorporated herein by reference.

A polypeptide of the invention comprising the above defined exposed and/or non-exposed mannose residues may-be obtained by in vitro glycosylation, e.g. utilizing available attachment groups on the wild-type or modified polypeptide. Chemically synthesized oligosaccharide structures can be attached to the polypeptide using a variety of different chemistries e.g. the chemistries employed for attachment of PEG to proteins, wherein the oligosaccharide is linked to a functional group, optionally via a short spacer (see the section entitled Conjugation to a Non-Oligosaccharide Macromolecular Moiety). The in vitro glycosylation can be carried out in a suitable buffer at pH 4-7 in protein concentrations of 0.5-2 mg/ml and a volume of 0.02-2 ml. The activated mannose compound is present in 2-200 fold molar excess, and reactions are incubated at 4-25° C. for periods of 0.1-3 hours. In vitro glycosylated GCB polypeptides are purified by dialysis and standard chromatographic techniques.

Other in vitro glycosylation methods are described, for example in WO 87/05330, by Aplin etl al., CRC Crit Rev. Biochem., pp.259-306, 1981. Furthermore, Doebber et al., J. Biol. Chem., 257, pp2193-2199, 1982, the contents of which are incorporated herein by reference, describe a convenient method for attaching a synthetic Man3Lys2 glycopeptide to lysine residues by in vitro glycosylation. However, coupling of a lysine residue may result in increased immunogenicity of the resulting polypeptide, and may not always be desireable for the present purpose.

Furthermore, in vitro glycosylation to protein- and peptide-bound Gln-residues can be carried out by transglutaminases (TGases). Transglutaminases catalyse the transfer of donor amine-groups to protein- and peptide-bound Gln-residues in a so-called cross-linking reaction. The donor-amine groups can be protein- or peptide-bound e.g. as the ε-amino-group in Lys-residues or it can be part of a small or large organic molecule. An example of a small organic molecule functioning as amino-donor in TGase-catalysed cross-linking is putrescine (1,4-diaminobutane). An example of a larger organic molecule functioning as amino-donor in TGase-catalysed cross-linking is an amine-containing PEG (Sato et al., Biochemistry 35, 1996, 13072-13080).

TGases, in general, are highly specific enzymes, and not every Gln-residues exposed on the surface of a protein is accessible to TGase-catalysed cross-linking to amino-containing substances. In order to render a protein susceptible to TGase-catalysed cross-linking reactions stretches of amino acid sequence known to function very well as TGase substrates are inserted at convenient positions in the amino acid sequence encoding a GCB polypeptide. Several amino acid sequences are known to be or to contain excellent natural TGase substrates e.g. substance P, elafin, fibrinogen, fibronectin, α₂-plasmin inhibitor, α-caseins, and β-caseins and may thus be inserted into and thereby constitute part of the amino acid sequence of a polypeptide of the invention.

Normally, the glycosylated polypeptide of the invention comprises 1-15 oligosaccharide moieties, such as 1-10 or 1-6 oligosachharide moieties.

The glycosylated polypeptide of the invention may further comprise at least one non-oligosaccharide macromolecular moiety, such as a polymer molecule, e.g. PEG, attached to an attachment group present in the parent polypeptide or having been introduced (as described in the section entitled “Conjugation to a non-oligosaccharide macromolecular moiety”).

Conjugation to a Non-Oligosaccharide Macromolecular Moiety

In the present application focus has been made to modify lysosomal enzyme and lysosomal enzyme activators by introduction of additional glycosylation sites. However, the invention is not limited to modification of glycosylation sites only. Also included in the invention is modification of amino acid residues constituting an attachment group for any other suitable (non-oligosaccharide) macromolecular moiety, in particular a polymer moiety such as PEG. It will be understood that the same principles for introducing/removing attachment groups for PEG etc apply as has been described above for introduction/removal of glycosylation site. In particular, in connection with introducing/removing in vitro glycosylation sites, since such sites may also function as attachment group for non-oligosaccharide macromolecular moieties such as PEG.

Accordingly, in one aspect the polypeptide of the invention is a lysosomal enzyme or lysosomal enzyme activator that comprises an amino acid sequence that differs from that of a parent enzyme or activator by at least one introduced and/or at least one removed amino acid residue comprising an attachment group for a non-oligosaccharide macromolecular moiety, the introduction and/or removal of the attachment group being done analogously to that described in the sections “Introduction of a glycosylation site” and “Removal of a glycosylation site”. Thus, for instance, the attachment group may be introduced into the mature part of the polypeptide or by means of a peptide addition on the basis of the same principles as those described above for introduction of a glycosylation site. The polypeptide according to this aspect is preferably a conjugated polypeptide comprising at least one non-oligosaccharide macromolecular moiety attached to the relevant attachment group. The conjugated polypeptide may further comprise at least one oligosaccharide moiety (e.g. as a consequence of in vivo or in vitro glycosylation). The polypeptide according to this embodiment may be any of the glycosylated polypeptides described herein, or may be one that does not contain an additional glycosylation site (relative to the parent polypeptide).

The type of macromolecular moiety is selected on the basis of the effect it is desired to provide. For instance, for shielding of epitopes and increasing serum half-life, a polymer such as PEG has been found useful. For increasing targeting to lysosomes the macromolecular moiety is preferably a phosholipid, a lipid or a mannose-containing compound.

The attachment group to which the macromolecular moiety is conjugated may be one which is present in the parent polypeptide, e.g. wtGCB, or may be one, which has been introduced into the amino acid sequence thereof and is thus not present in parent. Thereby, the polypeptide is boosted or otherwise altered in the content of the specific amino acid residues to which the macromolecular moiety of choice binds, whereby a more efficient, specific and/or extensive conjugation is achieved. For instance, when the total number of amino acid residues comprising an attachment group for the macromolecular moiety of choice is increased a greater proportion of the polypeptide molecule is shielded and thus a lower immune response will result. In most cases the introduction of an amino acid residue will be by way of substitution of an amino acid residue.

The position into which an amino acid residue comprising an attachment group is to be introduced is as described above for introduction of an in vitro glycosylation site. The amino acid residue comprising an attachment group for the macromolecular moiety is selected on the basis of the nature of the macromolecular moiety of choice and, in most instances, on the basis of the type of macromolecular moiety and the chemistry to be used for achieving the conjugation between the polypeptide and the macromolecular moiety. For instance, when the macromolecular moiety is a polymer molecule such as a polyethylene glycol or polyalkylene oxide derived molecule an amino acid residue comprising a suitable attachment group is normally selected from the group consisting of lysine, cysteine, aspartic acid, glutamic acid and arginine. When conjugation to a lysine residue is to be achieved a suitable activated molecule is, e.g., mPEG-SPA, mPEG-SCM, mPEG-BTC from Shearwater Polymers, Inc, SC-PEG from Enzon, Inc., tresylated niPEG as described in U.S. Pat. No. 5,880,255, or oxycarbonyl-oxy-N-dicarboxyimide-PEG (U.S. Pat. No. 5,122,614).

Preferably, the amino acid residue comprising an attachment group for the macromolecular moiety of choice is introduced into a position exposed on the surface of the parent polypeptide, in particular into a position which in the parent polypeptide is occupied by a charged residue such as an arginine, histidine, lysine, glutamic acid and/or aspartic acid residue or a position located between −4 and +4 amino acid residues from such charged amino acid residue.

For instance, when lysine comprises the attachment group, modification of a parent GCB polypeptide may be achieved as described for introducion and/or removal of in vitro glycosylation sites in GCB (section entitled “A GCB polypeptide of the invention”).

In a further embodiment, the polypeptide of the invention is one, wherein at least one amino acid residue comprising an attachment group for a macromolecular moiety has been removed (as compared to the parent GCB). By removing one or more amino acid residues comprising an attachment group for a macromolecular moiety of choice it is possible to avoid conjugation to the macromolecular moiety in parts of the polypeptide in which such conjugation is disadvantageous, e.g. in amino acid residue located at or near a functional site of the polypeptide. In particular in case of a polypeptide of the invention comprising one or more additional glycosylation sites, one or more amino acid residues comprising an attachment group for the non-oligosaccharide macromolecular moiety may be removed, if located at or within 4 amino acid residues of an O- or N-glycosylation site (in the primary sequence), since conjugation at such a site may result in inactivation or reduced activity of the resulting conjugate due to impaired receptor recognition.

In a further embodiment thepolypeptide of the invention differs from a parent polypeptide, e.g. GCB, in that at least one amino acid residue comprising an attachment group for a macromolecular moiety has been introduced into the sequence and at least one amino acid residue comprising an attachment group for the same macromolecular moiety and present in the parent polypeptide has been removed from the sequence. This embodiment is considered of particular interest for increasing the serum and/or functional in vivo half-life of a polypeptide of the invention and/or for shielding of epitopes, either present in the wildtype molecule, but more likely introduced by amino acid or glycosylation modifications of the wildtype molecule. For instance, by introducing and removing selected amino acid residues it is possible to ensure an optimal distribution of sites capable of attaching the macromolecular moiety of choice, which gives rise to a conjugated polypeptide in which the macromolecular moieties are placed so as to effectively shield epitopes and other surface parts of the polypeptide without causing too much structural disruption and thereby impair the function of the polypeptide.

As indicated above the non-oligosaccharide macromolecular moiety of the conjugated polypeptide according to this embodiment of the invention is preferably a polymer molecule. It may confer desirable properties to the polypeptide, in particular increased functional in vivo half-life and/or increased serum half-life, and/or reduced immunogenicity and/or reduced susceptibility to proteolytic degradation.

The polymer molecule to be coupled to the polypeptide may be any suitable polymer molecule, such as a natural or synthetic homo-polymer or heteropolymer, typically with a molecular weight in the range of 300-100,000 Da, such as 300-20,000 Da, more preferably in the range of 500-10,000 Da, even more preferably in the range of 500-5000 Da. Examples of homo-polymers include a polyol (i.e. poly-OH), a polyamine (i.e. poly-NH₂) and a polycarboxylic acid (i.e. poly-COOH). A hetero-polymer is a polymer, which comprises one or more different coupling groups, such as, e.g., a hydroxyl group and an amine group.

Examples of suitable polymer molecules include polymer molecules selected from the group consisting of polyalkylene oxide (PAO), including polyalkylene glycol (PAG), such as polyethylene glycol (PEG) and polypropylene glycol (PPG), branched PEGs, poly-vinyl alcohol (PVA), poly-carboxylate, poly-(vinylpyrolidone), polyethylene-co-maleic acid anhydride, polystyrene-co-malic acid anhydride, dextran including carboxymethyl-dextran, or any other biopolymer suitable for reducing immunogenicity and/or increasing functional in vivo half-life and/or serum half-life. Another example of a polymer molecule is human albumin or another abundant plasma protein. Generally, polyalkylene glycol-derived polymers are biocompatible, non-toxic, non-antigenic, non-immunogenic, have various water solubility properties, and are easily excreted from living organisms.

PEG is the preferred polymer molecule to be used, since it has only few reactive groups capable of cross-linking compared, e.g., to polysaccharides such as dextran, and the like. In particular, monofunctional PEG, e.g. methoxypolyethylene glycol (mPEG), is of interest since its coupling chemistry is relatively simple (only one reactive group is available for conjugating with attachment groups on the polypeptide). Consequently, the risk of cross-linking is eliminated, the resulting polypeptide conjugates are more homogeneous and the reaction of the polymer molecules with the polypeptide is easier to control.

To effect covalent attachment of the polymer molecule(s) to the polypeptide, the hydroxyl end groups of the polymer molecule must be provided in activated form, i.e. with reactive functional groups. Suitably activated polymer molecules are commercially available, e.g. from Shearwater Polymers, Inc., Huntsville, Ala., USA. Alternatively, the polymer molecules can be activated by conventional methods known in the art, e.g. as disclosed in WO 90/13540. Specific examples of activated linear or branched polymer molecules for use in the present invention are described in the Shearwater Polymers, Inc. 1997 and 2000 Catalogs (Functionalized Biocompatible Polymers for Research and pharmaceuticals, Polyethylene Glycol and Derivatives, incorporated herein by reference). Specific examples of activated PEG polymers include the following linear PEGs: NHS-PEG (e.g. SPA-PEG, SSPA-PEG, SBA-PEG, SS-PEG, SSA-PEG, SC-PEG, SG-PEG, and SCM-PEG), and NOR-PEG), BTC-PEG, EPOX-PEG, NCO-PEG, NPC-PEG, CDI-PEG, ALD-PEG, TRES-PEG, VS-PEG, IODO-PEG, and MAL-PEG, and branched PEGs such as PEG2-NHS and those disclosed in U.S. Pat. No. 5,932,462 and U.S. Pat. No. 5,643,575, both of which references are incorporated herein by reference. Furthermore, the following publications, incorporated herein by reference, disclose useful polymer molecules and/or PEGylation chemistries: U.S. Pat. Nos. 5,824,778, 5,476,653, WO 97/32607, EP 229,108, EP 402,378, U.S. Pat. Nos. 4,902,502, US 5,281,698, US 5,122,614, US 5,219,564, WO 92/16555, WO 94/04193, WO 94/14758, WO 94/17039, WO 94/18247, WO 94/28024, WO 95/00162, WO 95/11924, WO95/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, U.S. Pat. No. 5,736,625, WO 98/05363, EP 809 996, U.S. Pat. No. 5,629,384, WO 96/41813, WO 96/07670, U.S. Pat. No. 5,473,034, U.S. Pat. No. 5,516,673, EP 605 963, U.S. Pat. No. 5,382,657, EP 510 356, EP 400 472, EP 183 503 and EP 154 316.

The conjugation of the polypeptide and the activated polymer molecules is conducted by use of any conventional method, e.g. as described in the following references (which also describe suitable methods for activation of polymer molecules): R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y.). The skilled person will be aware that the activation method and/or conjugation chemistry to be used depends on the attachment group(s) of the polypeptide as well as the functional groups of the polymer (e.g. being amino, hydroxyl, carboxyl, aldehyde or sulfydryl). The PEGylation may be directed towards conjugation to all available attachment groups on the polypeptide (i.e. such attachment groups that are exposed at the surface of the polypeptide) or may be directed towards specific attachment groups, e.g. the N-terminal amino group (U.S. Pat. No. 5,985,265). Furthermore, the conjugation may be achieved in one step or in a stepwise manner (e.g. as described in WO 99/55377).

It will be understood that the PEGylation is designed so as to produce the optimal molecule with respect to the number of PEG-molecules attached, the size and form (e.g. whether they are linear or branched) of such molecules, and where in the polypeptide such molecules are attached. For instance, the molecular weight of the polymer to be used may be chosen on the basis of the desired effect to be achieved. For instance, if the primary purpose of the conjugation is to achieve a conjugate having a high molecule weight (e.g. to reduce renal clearance and thereby increase the serum and/or functional in vivo half-life) it is usually desirable to conjugate as few high Mw polymer molecules as possible to obtain the desired molecular weight. When a high degree of epitope or proteolytic site shielding is desirable this may be obtained by use of a sufficiently high number of low molecular weight polymer (e.g. with a molecular weight of about 5,000 Da) to effectively shield all or most epitopes of the polypeptide. For instance, 1-8, such as 1-4 such polymers may be used.

Normally, the polymer conjugation is performed under conditions aiming at reacting all available polymer attachment groups with polymer molecules. Typically, the molar ratio of activated polymer molecules to polypeptide is 1000-1, in particular 200-1, preferably 100-1, such as 10-1 or 5-1 in order to obtain optimal reaction.

It is also contemplated according to the invention to couple the polymer molecules to the polypeptide through a linker. Suitable linkers are well known to the skilled person. A preferred example is cyanuric chloride (Abuchowski et al., (1977), J. Biol. Chem., 252, 3578-3581; U.S. Pat. No. 4,179,337; Shafer et al., (1986), J. Polym. Sci. Polym. Chein. Ed., 24, 375-378. Subsequent to the conjugation residual activated polymer molecules are blocked according to methods known in the art, e.g. by addition of primary amine to the reaction mixture, and the resulting inactivated polymer molecules removed by a suitable method.

Properties of a Polypeptide of the Invention

Preferably, the polypeptide of the invention has at least one of the following properties relative to the parent polypeptide or a reference molecule, the properties being measured under comparable conditions:

Increased in vivo activity;

in vitro bioactivity which is at least 25%, such as at least 50% or at least 75% of that of the parent or reference polypeptide as measured under comparable conditions,

increased affinity for a mannose receptor, mannose-6-phosphate-receptor, or other carbohydrate receptors,

increased serum or functional in vivo half-life,

reduced renal clearance,

reduced immunogenicity,

increased resistance to proteolytic cleavage,

increased targeting to and/or uptake in phagocytic cells, such as macrophages or macrophage like cells or a suborganel compartment thereof (lysosomes) or other subpopulations of human cells (e.g. muscle cells, fibroblasts, etc.) of relevance for the specific polypeptide of the invention,

improved stability in production, improved shelf life, improved formulation, e.g. liquid formulation,

improved purification, improved solubility, and/or improved expression.

Improved properties are determined by conventional methods known in the art for determining such properties or as described herein.

Methods of Preparing a Polypeptide of the Invention

The invention further comprises a method of producing the present polypeptide comprising culturing a host cell transformed or transfected with a nucleotide sequence encoding the polypeptide under conditions permitting the expression of the polypeptide, and recovering the polypeptide from the culture.

The term “nucleotide sequence” is intended to indicate a consecutive stretch of two or more nucleotide molecules. The nucleotide sequence may be of genomic, cDNA, RNA, semisynthetic, synthetic origin, or any combinations thereof.

The terms “cell”, “host cell”, “cell line” and “cell culture” are used interchangeably herein and all such terms should be understood to include progeny resulting from growth or culturing of a cell. “Transformation” and “transfection” are used interchangeably to refer to the process of introducing DNA into a cell.

Apart from recombinant production, polypeptides of the invention may be produced, albeit less efficiently, by chemical synthesis or a combination of chemical synthesis and recombinant DNA technology.

The nucleotide sequence of the invention encoding a polypeptide of the invention may be constructed by isolating or synthesizing a nucleotide sequence encoding the relevant parent polypeptide (in the case of GCB for instance wt GCB with the amino acid sequence shown in SEQ ID NO: 1) and then changing the nucleotide sequence so as to effect introduction (i.e. insertion or substitution) or removal (i.e. deletion or substitution) of the relevant amino acid residue(s). The nucleotide sequence is conveniently modified by site-directed mutagenesis in accordance with well-known methods, e.g. as described in Nelson and Long, Analytical Biochemistry 180, 147-151, 1989.

Alternatively, the nucleotide sequence may be prepared by chemical synthesis, e.g. by using an oligonucleotide synthesizer, wherein oligonucleotides are designed based on the amino acid sequence of the desired polypeptide, and preferably selecting those codons that are favoured in the host cell in which the recombinant polypeptide will be produced. For example, several small oligonucleotides coding for portions of the desired polypeptide may be synthesized and assembled by PCR, ligation or ligation chain reaction (LCR). The individual oligonucleotides typically contain 5′ or 3′ overhangs for complementary assembly.

Once assembled (by synthesis, site-directed mutagenesis or another method), the nucleotide sequence encoding the polypeptide may be inserted into a recombinant vector and operably linked to control sequences necessary for expression of the polypeptide in the desired transformed host cell.

It should of course be understood that not all vectors and expression control sequences function equally well to express the nucleotide sequence encoding a polypeptide of the invention. Neither will all hosts function equally well with the same expression system. However, one of skill in the art may make a selection among these vectors, expression control sequences and hosts without undue experimentation. For example, in selecting a vector, the host must be considered because the vector must replicate in it or be able to integrate into the chromosome. The vector's copy number, the ability to control that copy number, and the expression of any other proteins encoded by the vector, such as antibiotic markers, should also be considered. In selecting an expression control sequence, a variety of factors should also be considered. These include, for example, the relative strength of the sequence, its controllability, and its compatibility with the nucleotide sequence encoding the polypeptide, particularly as regards potential secondary structures. Hosts should be selected by consideration of their compatibility with the chosen vector, the toxicity of the product coded for by the nucleotide sequence, their secretion characteristics, their ability to fold the polypeptide correctly, their fermentation or culture requirements, and the ease of purification of the products coded for by the nucleotide sequence.

The recombinant vector may be an autonomously replicating vector, i.e. a vector which exists as an extrachromosomal entity, the replication of which is independent of chromosomal replication, e.g. a plasmid. Alternatively, the vector is one which, when introduced into a host cell, is integrated into the host cell genome and replicated together with the chromosome(s) into which it has been integrated.

The vector is preferably an expression vector, in which the nucleotide sequence encoding the polypeptide of the invention is operably linked to additional segments required for transcription of the nucleotide sequence. The vector is typically derived from plasmid or viral DNA. A number of suitable expression vectors for expression in the host cells mentioned herein are commercially available or described in the literature. Useful expression vectors for eukaryotic hosts, include, for example, vectors comprising expression control sequences from SV40, bovine papilloma virus, adenovirus and cytomegalovirus. Specific vectors are, e.g., pcDNA3.1(+)\Hyg (Invitrogen, Carlsbad, Calif., USA) and pCI-neo (Stratagene, La Jolla, Calif., USA). Useful expression vectors for yeast cells include the 2μ plasmid and derivatives thereof, the POT1 vector (U.S. Pat. No. 4,931,373), the pJSO37 vector described in (Okkels, Ann. New York Acad. Sci. 782, 202-207, 1996) and pPICZ A, B or C (Invitrogen, Carlsbad, Calif., USA). Useful vectors for insect cells include pVL941, pBG311 (Cate et al., “Isolation of the Bovine and Human Genes for Mullerian Inhibiting Substance And Expression of the Human Gene In Animal Cells”, Cell, 45, pp. 685-98 (1986), pBluebac 4.5 and pMelbac (both available from Invitrogen, Carlsbad, Calif., USA).

Other vectors for use in this invention include those that allow the nucleotide sequence encoding the polypeptide to be amplified in copy number. Such amplifiable vectors are well known in the art. They include, for example, vectors able to be amplified by DHFR amplification (see, e.g., Kaufman, U.S. Pat. No. 4,470,461, Kaufman and Sharp, “Construction Of A Modular Dihydrafolate Reductase cDNA Gene: Analysis Of Signals Utilized For Efficient Expression”, Mol. Cell. Biol., 2, pp. 1304-19 (1982)) and glutamine synthetase (“GS”) amplification (see, e.g., U.S. Pat. No. 5,122,464 and EP 338,841).

The recombinant vector may further comprise a DNA sequence enabling the vector to replicate in the host cell in question. An example of such a sequence (when the host cell is a mammalian cell) is the SV40 origin of replication. When the host cell is a yeast cell, suitable sequences enabling the vector to replicate are the yeast plasmid 2μ replication genes REP 1-3 and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g. ampicillin, kanamycin, tetracyclin, chloramphenicol, neomycin, hygromycin or methotrexate. For filamentous fungi, selectable markers include amdS, pyrG, arcB, niaD, sC.

The term “control sequences” is defined herein to include all components, which are necessary or advantageous for the expression of the polypeptide of the invention. Each control sequence may be native or foreign to the nucleic acid sequence encoding the polypeptide. Such control sequences include, but are not limited to, a leader, polyadenylation sequence, propeptide sequence, promoter, enhancer or upstream activating sequence, signal peptide sequence, and transcription terminator. At a minimum, the control sequences include a promoter operably linked to the nucleotide sequence encoding the polypeptide.

“Operably linked” refers to the covalent joining of two or more nucleotide sequences, by means of enzymatic ligation or otherwise, in a configuration relative to one another such that the normal function of the sequences can be performed. For example, the nucleotide sequence encoding a presequence or secretory leader is operably linked to a nucleotide sequence for a polypeptide if it is expressed as a preprotein that participates in the secretion of the polypeptide: a promoter or enhancer is operably linked to a coding sequence if it affects the transcription of the sequence; a ribosome binding site is operably linked to a coding sequence if it is positioned so as to facilitate translation. Generally, “operably linked” means that the nucleotide sequences being linked are contiguous and, in the case of a secretory leader, contiguous and in reading phase. Linking is accomplished by ligation at convenient restriction sites. If such sites do not exist, then synthetic oligonucleotide adaptors or linkers are used, in conjunction with standard recombinant DNA methods.

A wide variety of expression control sequences may be used in the present invention. Such useful expression control sequences include the expression control sequences associated with structural genes of the foregoing expression vectors as well as any sequence known to control the expression of genes of prokaryotic or eukaryotic cells or their viruses, and various combinations thereof.

Examples of suitable control sequences for directing transcription in mammalian cells include the early and late promoters of SV40 and adenovirus, e.g. the adenovirus 2 major late promoter, the MT-1 (metallothionein gene) promoter, the human cytornegalovirus immediate-early gene promoter (CMV), the human elongation factor 1α (EF-1α) promoter, the Drosophila minimal heat shock protein 70 promoter, the Rous Sarcoma Virus (RSV) promoter, the human ubiquitin C (UbC) promoter, the human growth hormone terminator, SV40 or adenovirus E1b region polyadenylation signals and the Kozak consensus sequence (Kozak, M. J. Mol Biol Aug. 20, 1987;196(4):947-50).

In order to improve expression in mammalian cells a synthetic intron may be inserted in the 5′ untranslated region of the nucleotide sequence encoding the polypeptide of the invention. An example of a synthetic intron is the synthetic intron from the plasmid pCI-Neo (available from Promega Corporation, WI, USA).

Examples of suitable control sequences for directing transcription in insect cells include the polyhedrin promoter, the P10 promoter, the Autographa californica polyhedrosis virus basic protein promoter, the baculovirus immediate early gene 1 promoter and the baculovirus 39K delayed-early gene promoter, and the SV40 polyadenylation sequence.

Examples of suitable control sequences for use in yeast host cells include the promoters of the yeast α-mating system, the yeast triose phosphate isomerase (TPI) promoter, promoters from yeast glycolytic genes or alcohol dehydogenase genes, the ADH2-4c promoter and the inducible GAL promoter.

Examples of suitable control sequences for use in filamentous fungal host cells include the ADH3 promoter and terminator, a promoter derived from the genes encoding Aspergillus oryzae TAKA amylase triose phosphate isomerase or alkaline protease, an A. niger α-amylase, A. niger or A. nidulans glucoamylase, A. nidulans acetamidase, Rhizomucor miehei aspartic proteinase or lipase, the TPI1 terminator and the ADH3 terminator.

The nucleotide sequence of the invention encoding a GCB polypeptide, whether prepared by site-directed mutagenesis, synthesis or other methods, may or may not also include a nucleotide sequence that encode a signal peptide. The signal peptide is present when the polypeptide is to be secreted from the cells in which it is expressed. Such signal peptide, if present, should be one recognized by the cell chosen for expression of the polypeptide. The signal peptide may be homologous (e.g. be that normally associated with human GCB) or heterologous (i.e. originating from another source than human GCB) to the polypeptide or may be homologous or heterologous to the host cell, i.e. a signal peptide normally expressed from the host cell or one which is not normally expressed from the host cell. Accordingly, the signal peptide may be prokaryotic, e.g. derived from a bacterium, or eukaryotic, e.g. derived from a mammalian, or insect, filamentous fungal or yeast cell.

The presence or absence of a signal peptide will, e.g., depend on the expression host cell used for the production of the polypeptide, the protein to be expressed (whether it is an intracellular or extracelluar protein) and whether it is desirable to obtain secretion. For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase. For use in insect cells, the signal peptide may conveniently be derived from an insect gene (cf. WO 90/05783), such as the lepidopteran Manduca sexta adipokinetic hormone precursor, (cf. U.S. Pat. No. 5,023,328), the honeybee melittin (Invitrogen, Carlsbad, Calif., USA), ecdysteroid UDP glucosyltransferase (egt) (Murphy et al., Protein Expression and Purification 4, 349-357 (1993) or human pancreatic lipase (hpl) (Methods in Enzymology 284, pp. 262-272, 1997).

A preferred signal peptide for use in mammalian cells is that of human GCB apparent from the examples hereinafter (when the polypeptide is a GCB polypeptide) or the murine Ig kappa light chain signal peptide (Coloma, M (1992) J. Imm. Methods 152:89-104). For use in yeast cells suitable signal peptides have been found to be the α-factor signal peptide from S. cereviciae. (cf. U.S. Pat. No. 4,870,008), the signal peptide of mouse salivary amylase (cf. 0. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), and the yeast aspartic protease 3 (YAP3) signal peptide (cf. M. Egel-Mitani et al., Yeast 6, 1990, pp. 127-137).

Any suitable host may be used to produce the polypeptide of the invention, including bacteria, fungi (including yeasts), plant, insect, mammal, or other appropriate animal cells or cell lines, as well as transgenic animals or plants. When a non-glycosylating organism such as E. coli is used, and the polypeptide of the invention is to be a glycosylated polypeptide, the expression in E. coli is preferably followed by suitable in vitro glycosylation.

Examples of bacterial host cells include grampositive bacteria such as strains of Bacillus, e.g. B. brevis or B. subtilis, Pseudomonas or Streptomyces, or gramnegative bacteria, such as strains of E. coli. The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278).

Examples of suitable filamentous fungal host cells include strains of Aspergillus, e.g. A. oryzae, A. niger, or A. nidulans, Fusarium or Trichoderma. Fungal cells may be transformed by a process involving protoplast formation, transformation of the protoplasts, and regeneration of the cell wall in a manner known per se. Suitable procedures for transformation of Aspergillus host cells are described in EP 238 023 and U.S. Pat. No. 5,679,543. Suitable methods for transforming Fusarium species are described by Malardier et al., 1989, Gene 78: 147-156 and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

The host cell is preferably selected from a group of host cells capable of generating the desired glycosylation of the polypeptide for improved lysosomal activity. Thus, the host cell may advantageously be selected from a yeast cell, insect cell, or mammalian cell.

Examples of suitable yeast host cells include strains of Saccharomyces, e.g. S. cerevisiae, Schizosaccharomyces, Klyveromyces, Pichia, such as P. pastoris or P. methanolica, Hansenula, such as H. polymorpha or yarrowia. Of particular interest are yeast glycosylation mutant cells, e.g. derived from S. cereviciae, P. pastoris or Hansenula spp. (e.g. the S. cereviciae glycosylation mutants och1, ochi mnm1 or och1 mnm1 alg3 described by Nagasu et al. Yeast 8, 535-547, 1992 and Nakanisho-Shindo et al. J. Biol. Chem. 268, 26338-26345, 1993). Methods for transforming yeast cells with heterologous DNA and producing heterologous polypeptides therefrom are disclosed by Clontech Laboratories, Inc, Palo Alto, Calif., USA (in the product protocol for the Yeastmaker Yeast Tranformation System Kit), and by Reeves et al., FEMS Microbiology Letters 99 (1992) 193-198, Manivasakam and Schiestl, Nucleic Acids Research, 1993,. Vol. 21, No. 18, pp. 4414-4415 and Ganeva et al., FEMS Microbiology Letters 121 (1994) 159-164.

Examples of suitable insect host cells include a Lepidoptora cell line, such as Spodoptera frugiperda (Sf9 or Sf21) or Trichoplusia ni cells (High Five) (U.S. Pat. No. 5,077,214). Transformation of insect cells and production of heterologous polypeptides therein may be performed as described by Invitrogen, Carlsbad, Calif., USA.

Examples of suitable mammalian host cells include Chinese hamster ovary (CHO) cell lines, (e.g CHO-K1; ATCC CCL-61), Green Monkey cell lines (COS) (e.g. COS 1 (ATCC CRL-1650), COS 7 (ATCC CRL-1651)); mouse cells (e.g. NS/O), Baby Hamster Kidney (BHK) cell lines (e.g. ATCC CRL-1632 or ATCC CCL-10), and human cells (e.g. HEK 293 (ATCC CRL-1573)), as well as plant cells in tissue culture. Additional suitable cell lines are known in the art and available from public depositories such as the American Type Culture Collection, Rockville, Md. Of particular interest for the present purpose are a mammalian glycosylation mutant cell line, such as CHO-LEC1, CHOL-LEC2 or CHO-LEC18 (CHO-LEC1: Stanley et al. Proc. Natl. Acad. USA 72, 3323-3327, 1975 and Grossmann et al., J. Biol. Chem. 270, 29378-29385, 1995, CHO-LEC18: Raju et al. J. Biol. Chem. 270, 30294-30302, 1995).

In a specific aspect the invention relates to a glycosylation mutant derived from yeast, e.g. Saccharomyces cerevisiae, Pichia pastoris or Hansenula spp. or a mammalian glycosylation mutant cell line as mentioned above comprising a heterologous nucleotide sequence encoding a lysosomal enzyme or a lysosmal enzyme activator, in particular GCB polypeptide. The lysosomal enzyme may be a wt enzyme or a polypeptide as described in the present invention. Likewise the activator may be a wt activator or a polypeptide as described herein. The mammalian glycosylation mutant cell line is preferably CHO-LEC1.

Methods for introducing exogeneous DNA into mammalian host cells include calcium phosphate-mediated transfection, electroporation, DEAE-dextran mediated transfection, liposome-mediated transfection, viral vectors and the transfection method described by Life Technologies Ltd, Paisley, UK using Lipofectamin 2000. These methods are well known in the art and e.g. described by Ausbel et al. (eds.), 1996, Current Protocols in Molecular Biology, John Wiley & Sons, New York, USA. The cultivation of mammalian cells are conducted according to established methods, e.g. as disclosed in (Animal Cell Biotechnology, Methods and Protocols, Edited by Nigel Jenkins, 1999, Human Press Inc, Totowa, N.J., USA and Harrison M A and Rae I F, General Techniques of Cell Culture, Cambridge University Press 1997).

In the production methods of the present invention, the cells are cultivated in a nutrient medium suitable for production of the polypeptide using methods known in the art. For example, the cell may be cultivated by shake flask cultivation, small-scale or large-scale fermentation (including continuous, batch, fed-batch, or solid state fermentations) in laboratory or industrial fermenters performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The resulting polypeptide may be recovered by methods known in the art. For example, the polypeptide may be recovered from the nutrient medium by conventional procedures including, but not limited to, centrifugation, filtration, extraction, spray drying, evaporation, or precipitation.

The polypeptides may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J-C Janson and Lars Ryden, editors, VCH Publishers, New York, 1989). Specific methods for purifying GCB polypeptides are disclosed in U.S. Pat. No. 5,236,838 and Osiecki-Newman et al., Enzyme 35, 147-153, 1986.

Other Methods of the Invention

Introduction of Glycosylation Sites

While glycosylation sites (or other attachment groups as described herein) can be introduced by a strictly directed approach (e.g. based on site-directed mutagenesis), it is also possible to use a random approach based on random mutagenesis, recombination, shuffling, or any other technology. For instance, a nucleotide sequence encoding a polypeptide of the invention (optionally including an N- or C-terminal peptide addition and/or being a chimeric polypeptide) can be constructed from two or more nucleotide sequences encoding the polypeptide, the sequences being sufficiently homologous to allow recombination between the sequences, in particular in the part thereof where the glycosylation site or other attachment group (or peptide addition) is to be introduced. The combination of nucleotide sequences or sequence parts is conveniently conducted by methods known in the art, for instance methods which involve homologous cross-over such as disclosed in U.S. Pat. No. 5,093,257, or methods which involve gene shuffling, i.e., recombination between two or more homologous nucleotide sequences resulting in new nucleotide sequences having a number of nucleotide alterations when compared to the starting nucleotide sequences. In order for homology based nucleic acid shuffling to take place the relevant parts of the nucleotide sequences are preferably at least 50% identical, such as at least 60% identical, more preferably at least 70% identical, such as at least 80% identical. The recombination can be performed in vitro or in vivo. Examples of suitable in vitro gene shuffling methods are disclosed by Stemmer et al (1994), Proc. Natl. Acad. Sci. USA; vol. 91, pp. 10747-10751; Stemmer (1994), Nature, vol. 370, pp. 389-391; Smith (1994), Nature vol. 370, pp. 324-325; Zhao et al., Nat. Biotechnol. March 1998; 16(3): 258-61; Zhao H. and Arnold, F B, Nucleic Acids Research, 1997, Vol. 25. No. 6 pp. 1307-1308; Shao et al., Nucleic Acids Research Jan 15, 1998; 26(2): pp. 681-83; and WO 95/17413. Example of a suitable in vivo shuffling method is disclosed in WO 97/07205.

Furthermore, a nucleotide sequence encoding a polypeptide of the invention can be constructed by preparing a randomly mutagenized library, conveniently prepared by subjecting a nucleotide sequence encoding the polypeptide (or, when relevant, the peptide addition) to random mutagenesis to create a large number of mutated nucleotide sequences. While the random mutagenesis can be entirely random, both with respect to where in the nucleotide sequence the mutagenesis occurs and with respect to the nature of mutagenesis, it is preferably conducted so as to randomly mutate only the part of the sequence in which a glycosylation site or other attachment group is to be introduced or the part encoding the peptide addition. The random mutagenesis can be directed towards introducing certain types of amino acid residues, in particular amino acid residues containing a glycosylation site or other attachment group, at random into the polypeptide molecule or at random into a peptide addition part thereof. Besides substitutions, random mutagenesis can also cover random introduction of insertions or deletions. Preferably, the insertions are made in reading frame, e.g., by performing multiple introduction of three nucleotides as described by Hallet et al., Nucleic Acids Res. 1997, 25(9):1866-7 and Sondek and Shrotle, Proc Natl. Acad. Sci USA 1992, 89(8):3581-5.

The random mutagenesis (either of the whole nucleotide sequence or a part thereof, e.g. the part encoding the peptide addition) can be performed by any suitable method. For example, the random mutagenesis is performed using a suitable physical or chemical mutagenizing agent, a suitable oligonucleotide, PCR generated mutagenesis or any combination of these mutagenizing agentsand/or other methods according to state of the art technology, e.g. as disclosed in WO 97/07202.

Error prone PCR generated mutagenesis, e.g. as described by J. O. Deshler (1992), GATA 9(4): 103-106 and Leung et al., Technique (1989) Vol. 1, No. 1, pp. 11-15, is particularly useful for mutagenesis of longer peptide stretches (corresponding to nucleotide sequences containing more than 100 bp) or entire genes, and are preferably performed under conditions that increase the misincorporation of nucleotides.

Random mutagenesis based on doped or spiked oligonucleotides or by specific sequence oligonucleotides, is of particular use for mutagenesis of the part of the nucleotide sequence encoding the peptide addition.

Random mutagenesis of the part of the nucleotide sequence encoding the peptide addition can be performed using PCR generated mutagenesis, in which one or more suitable oligonucleotide primers flanking the area to be mutagenized are used. In addition, doping or spiking with oligonucleotides can be used to introduce mutations so as to remove or introduce glycosylation sites. State of the art knowledge and computer programs (e.g. as described by Siderovski D P and Mak T W, Comput. Biol. Med. (1993) Vol. 23, No. 6, pp. 463-474 and Jensen et al. Nucleic Acids Research, 1998, Vol. 26, No. 3) can be used for calculating the most optimal nucleotide mixture for a given amino acid preference. The oligonucleotides can be incorporated into the nucleotide sequence encoding the peptide addition by any published technique using e.g. PCR, LCR or any DNA polymerase or ligase.

According to a convenient PCR method the nucleotide sequence encoding the polypeptide of the invention or, e.g., a peptide addition thereof, is used as a template and, e.g., doped or specific oligonucleotides are used as primers. In addition, cloning primers localized outside the targetted region can be used. The resulting PCR product can either directly be cloned into an appropriate expression vector or gel purified and amplified in a second PCR reaction using the cloning primers and cloned into an appropriate expression vector.

In addition to the random mutagenesis methods described herein, it is occasionally useful to employ site specific mutagenesis techniques to modify one or more selected amino acids in the polypeptide, in particular to optimise the polypeptide with respect to the number of glycosylation sites.

Furthermore, random elongation mutagenesis as described by Matsuura et al, Nature Biotechnology, 1999, Vol. 17, 58-61, can be used to construct a nucleotide sequence encoding the polypeptide of the invention having a C-terminal peptide addition. Construction of a nucleotide sequence encoding the polypeptide of the invention having an N-terminal peptide addition can be constructed in an analogous way.

Also, the methods disclosed in WO 97/04079, the contents of which are incorporated herein by reference, can be used for constructing a nucleotide sequence encoding a polypeptide of the invention.

The nucleotide sequence(s) or nucleotide sequence region(s) to be mutagenized is typically present on a suitable vector such as a plasmid or a bacteriophage, which as such is incubated with or otherwise exposed to the mutagenizing agent. The nucleotide sequence(s) to be mutagenized can also be present in a host cell either by being integrated into the genome of said cell or by being present on a vector harboured in the cell. Alternatively, the nucleotide sequence to be mutagenized is in isolated form. The nucleotide sequence is preferably a DNA sequence such as a cDNA, genomic DNA or synthetic DNA sequence.

Subsequent to the incubation with or exposure to the mutagenizing agent, the mutated nucleotide sequence, normally in amplified form, is expressed by culturing a suitable host cell carrying the nucleotide sequence under conditions allowing expression to take place. The host cell used for this purpose is one, which has been transformed with the mutated nucleotide sequence(s), optionally present on a vector, or one which carried the nucleotide sequence during the mutagenesis, or any kind of gene library.

Constructing a Peptide Addition

As a non-limiting example an N-terminal peptide addition containing N-glycosylation sites can be designed on the basis of the following formula:

Y¹(NXT/S)Y²(NXT/S)_{z}Y³-P,

wherein each of Y¹, Y² and Y³ independently is absent or 1, 2, 3 or 4 amino acid residues of any type, X a single amino acid residue of any type except for proline, Z any integer between 0 and 6, T/S a threonine or serine residue, preferably a threonine residue, and N is an asparagine residue and P is the lysosomal enzyme or activator to be modified.

In a first step about 10 different muteins are made that has the above formula. For instance, the about 10 muteins are designed on the basis that each of Y¹, Y² and Y³ independently is 1 or 2 alanine residues or is absent, Z any integer between 0 and 5, T/S threonine, and X alanine. Based on, e.g., in vitro bioactivity and half-life results obtained with these muteins (or any other relevant property), optimal number(s) of amino acids and glycosylation(s) can be determined and new muteins can be constructed based on this information. The process is repeated until an optimal glycosylated polypeptide is obtained.

Alternatively, random mutagenesis may be used for creating N-terminally extended polypeptides. For instance, a random mutagenized library is made on the basis of the above formula. Doped oligonucleotides are synthesized coding for one amino acid residue in position X (the amino acid residue being different from proline), each of Y¹, Y², and Y³ independently is 0, 1 or 2 amino acid residues of any type, Z is 2 and T is threonine and used for constructing the random mutagenized library.

As another non-limiting example an N-terminal peptide addition containing an in vitro glycosylation site can be designed on the basis of the following formula (using a lysine residue as an example of such site):

Y¹(K)Y²(K)_{z}Y³-P,

wherein each of Y¹, Y² and Y³ independently is 0, 1, 2, 3 or 4 amino acid residues of any type except lysine, Z an integer between 0 and 6, K lysine, and P is the lysosomal enzyme or activator.

In a first step about 10 different muteins are made that has the above formula. For instance, the about 10 muteins are designed on the basis that each of Y¹, Y² and Y³ independently is 1 or 2 alanine residues or is absent, Z any integer between 0 and 5, and X alanine. The muteins are then glycosylated with a suitable oligosaccharide moiety. Based on, e.g., in vitro bioactivity and half-life results obtained with these muteins (or any other relevant property), optimal number(s) of amino acids and glycosylation sites can be determined and new muteins can be constructed based on this information. The process is repeated until an optimal glycosylated polypeptide is obtained.

Alternatively, random mutagenesis may be performed by making a random mutagenized library based on the above formula. Doped oligonucleotides are synthesized coding for one amino acid residue in position X (expect proline) and each of Y¹, Y² and Y³ independently is 0, 1 or 2 amino acid residues of any type, and Z is 2 and used for constructing the random mutagenized library.

It will be understood that the above design schemes are intended for illustration purposes only and that a person skilled in the art will be aware of alternative useful routes for design of peptide addition. Furthermore, it will be understood that peptide additions with other attachment groups can be designed in an analogous way.

Furthermore, a nucleotide sequence encoding a polypeptide of the invention comprising an N- or C-terminal peptide addition can be prepared by a method comprising

a) subjecting a nucleotide sequence encoding the parent polypeptide to elongation mutagenesis,

b) expressing the mutated nucleotide sequence obtained in step a) in a suitable host cell to obtain an in vivo glycosylated polypeptide or subjecting the expressed polypeptide to in vitro glycosylation or conjugation to a non-oligosaccharide macromolecular moiety, as appropriate,

c) selecting glycosylated and/or conjugated polypeptides comprising at least one oligosaccharide or non-oligosaccharide macromolecular moiety attached to the peptide addition part of the polypeptide, and

d) isolating a nucleotide sequence encoding the polypeptide part of conjugates selected in step c).

In the present context the term “elongation mutagenesis” is intended to indicate any manner in which the nucleotide sequence encoding the parent polypeptide can be extended to further encode a peptide addition as described herein above. For instance, a nucleotide sequence encoding a peptide addition of a suitable length may be synthesized and fused to a nucleotide sequence encoding the polypeptide. The resulting fused nucleotide sequence may then be subjected to further modification by any suitable method, e.g. one which involves gene shuffling, other recombination between nucleotide sequences, random mutagenesis, random elongation mutagenesis or any combination of these methods (as described in the immediately preceding section).

The expression and conjugation steps are conducted as described in further detail elsewhere in the present application, and the selection step c) using any suitable method available in the art.

In one embodiment the above method further comprises screening conjugates resulting from step b) for at least one improved property, in particular any of those improved properties listed herein, one step prior to the selection step, and wherein the selection step c) further comprises selecting conjugates having such improved property.

Furthermore, in the above method the elongation mutagenesis can be conducted so as to enrich for codons encoding an amino acid residue comprising an attachment group for the oligosaccharide or non-oligosaccharide macromolecular moiety, in particular an in vivo glycosylation site.

Usually, when a polypeptide conjugate has been selected in a screening step of a method of the invention the nucleotide sequence encoding the polypeptide part of the conjugate is isolated and used for expression of larger amounts of the polypeptide. The amino acid sequence of the resulting polypeptide is determined and the polypeptide is subjected to conjugation in a larger scale. Subsequently, the polypeptide conjugate is assayed with respect to the property to be improved.

Assays for Biological Activity

Secondary screening can be performed to characterize the binding and uptake of the present polypeptides in macrophages. This is illustrated herein for GCB polypeptides, but a similar approach can be used for testing properties of other lysosomal enzymes.

It has been shown that GCB is taken up primarily by macrophages through the macrophage mannose receptor. Though many macrophage cell lines do not express functional macrophage mannose receptors, the murine macrophage cell line J774E has been found positive for this receptor (Blum et al., 1991, Carbohydr.Res 213, 145-153;). The uptake can either be measured by radioactively labelled GCB polypeptide or, as preferred, by enzyme activity assays on lyzed cells after uptake of the polypeptide (The combined uptake/activity assay is described in further detail in the examples section herein).

As an alternative to the murine macrophage cell line J774E, peritoneal macrophages can be isolated 6-8 weeks old BALB/CBYJ mice and used for studying the uptake of radioactively labelled GCB polypeptides (or the combined uptake/enzyme-activity assay).

In a further aspect the invention relates to an assay method for measuring the efficiency of cellular uptake of a GCB polypeptide into cultured macrophage cells, the method comprising culturing J774E Murine macrophage cells in a medium containing the GCB polypeptide for a sufficient period of time allowing for uptake of the GCB polypeptide, lysing said cells in the presence of a buffer containing a substrate for the GCB polypeptide, and measuring the amount of enzyme activity in the lysate.

The GCB to be assayed can be any GCB polypeptide, in particular a wtGCB or a functional fragment or variant thereof. In particular, the GCB polypeptide to be assayed may be a polypeptide of the present invention. In the method according to this aspect, a preferred substrate is para-nitrophenyl-glucopyranoside or 4-methylumbelliferyl-glucopyranoside.

The pharmacokinetics and dynamics of the present polypeptides may be studied to select for such polypeptides that exhibit a longer functional in vivo half-life in order to ensure infrequent dosing and prevent the low plasma levels seen with the currently available GCBs. The pharmacokinetics is studied by intravenous administration of the present polypeptides and thereafter determination of plasma clearance and cell specific distribution in liver and spleen by utilizing the GCB Activity Assay. Friedmann et al.,1999, Blood, 93; 2807-2816, have published a protocol to separate phagocytic Kupfer cells from other liver endothelial cells and thereafter study the cell specific uptake of administered GCB. Also, a suitable method is disclosed in the Methods section herein. Preferred polypeptides should either have slower plasma or lysosomal clearance and/or an improved lysosomal uptake.

Therapeutic Utility

While the polypeptide of the invention may be useful in the treatment of various types of diseases and disorders, it is presently contemplated to be of particular utility for substitution therapy in the prevention or treatment of a lysosomal storage disease treatable by the lysosomal enzyme of the polypeptide. When the polypeptide of the invention is a GCB, SapC or SapA polypeptide, the disease to be treated is preferably Gaucher's disease, in particular the Type 1 Gaucher's disease. Thus, in a preferred aspect, the present invention relates to the use of a GCB, SapC or SapA polypeptide of the invention for the manufacture of a medicament for the prevention or treatment of Gaucher's disease. Furthermore, the invention relates to a method of treating Gaucher's disease by administering, to a patient in need thereof, an effective amount of the GCB or SapC polypeptide, or a pharmaceutical composition of the invention. Analogously, when the polypeptide of the invention is alpha-galactosidase or SapB, it may be used in the treatment of Fabry's disease, when ceremidase or SapD, it may be used in the treatment of Farber's disease, when beta-galactosidase it may be used in the treatment of Gₘ₁ gangliosidosis, when beta-hexosaminidase or GM-2 activator, it may be used in the treatment of Tay-Sachs dieases, when sphingomyelinase in Niemann-Pick disase, when alpha-N-acetylgalactosaminidase for the treatment of Sly syndrome, when iduronidase for the treatment of Huler/Scheie syndrome, when galactocerebrosidase for the treatment of Batten disease, and when alpha-glucosidase for Pombe's disease.

While the polypeptide of the invention is anticipated to exhibit therapeutic utility for the same purpose, it is believed that, due to the improved lysosomal activity of the polypeptide, it may be administered in dosages that are lower than with the current treatment. For GCB, the recommended dosage by the manufacturer is 60 units/kg body weight/2 weeks. The GCB polypeptide of the invention may therefore be administered at a dose approximately paralleling that employed in therapy with human GCB such as Cerezyme™, or a lower dose and/or less frequently than Cerezyme™. The exact dose to be administered depends on the circumstances. Normally, the dose should be capable of preventing or lessening the severity or spread of the condition or indication being treated. It will be apparent to those of skill in the art that an effective amount of a polypeptide or composition of the invention depends, inter alia, upon the disease, the dose, the administration schedule, whether the polypeptide or composition is administered alone or in conjunction with other therapeutic agents, the serum half-life of the compositions, and the general health of the patient.

The polypeptide of the invention is preferably administered in a composition including a pharmaceutically acceptable carrier or excipient. “Pharmaceutically acceptable” means a carrier or excipient that does not cause any untoward effects in patients to whom it is administered. Such pharmaceutically acceptable carriers and excipients are well known in the art.

The polypeptide of the invention can be formulated into pharmaceutical compositions by well-known methods. Suitable formulations are described by Remington's Pharmaceutical Sciences by E. W. Martin and U.S. Pat. No. 5,183,746.

The pharmaceutical composition of the polypeptide of the invention may be formulated in a variety of forms, including liquid, gel, lyophilized, or any other suitable form. The preferred form will depend upon the particular indication being treated and will be apparent to one of skill in the art.

The pharmaceutical composition containing the polypeptide of the invention may be administered orally, intravenously, intramuscularly, intraperitoneally, intradermally, subcutaneously, by inhalation, or in any other acceptable manner, e.g. using PowderJect or ProLease technology. The preferred mode of administration will depend upon the particular indication being treated and will be apparent to one of skill in the art.

The pharmaceutical composition of the invention may be administered in conjunction with other therapeutic agents. These agents may be incorporated as part of the same pharmaceutical composition or may be administered separately from the polypeptide of the invention, either concurrently or in accordance with any other acceptable treatment schedule. For instance, when the polypeptide is a lysosomal enzyme such agent may be an activator thereof. When the lysosomal enzyme is GCB, SapC and/or SapA is one example of such agent. When the lysosomal enzyme is arylsulphatase A, SapB is an example of such agent. When the lysosomal enzyme is alpha-galactoisdase, SapB and/or SapD is an example of such agent. When the lysosomal enzyme is hexosaminidase, GM-2 activator is an example of such agent.

Also contemplated is the use of a nucleotide sequence encoding a polypeptide of the invention in gene therapy applications. In particular, it may be of interest to use a nucleotide sequence encoding a polypeptide having at least one introduced in vivo glycosylation site. The glycosylation of the polypeptides is thus achieved during the course of the gene therapy, i.e. after expression of the nucleotide sequence in the human body.

Both in vitro and in vivo gene therapy methodologies are contemplated. Several methods for transferring potentially therapeutic genes to defined cell populations-are known. For further reference see, e.g., Mulligan, “The Basic Science Of Gene Therapy”. Science, 260, pp. 926-31 (1993). These methods include:

Direct gene transfer, e.g., as disclosed by Wolff et al., “Direct Gene transfer Into Mouse Muscle In vivo”, Science 247, pp. 1465-68 (1990);

Liposome-mediated DNA transfer, e.g., as disclosed by Caplen et al., “Liposome-mediated CFTR Gene Transfer to the Nasal Epithelium Of Patients With Cystic Fibrosis” Nature Med., 3, pp. 39-46 (1995); Crystal, “The Gene As A Drug”, Nature Med., 1, pp.-1-5-17 (1995); Gao and Huang, “A Novel Cationic Liposome Reagent For Efficient Transfection of Mammalian Cells”, Biochem.Biophys Res. Comm., 179, pp. 280-85 (1991);

Retrovirus-mediated DNA transfer, e.g., as disclosed by Kay et al., “In vivo Gene Therapy of Hemophilia B: Sustained Partial Correction In Factor IX-Deficient Dogs”, Science, 262, pp. 117-19 (1993); Anderson, “Human Gene Therapy”, Science, 256, pp. 808-13(1992);

DNA Virus-mediated DNA transfer. Such DNA viruses include adenoviruses (preferably Ad-2 or Ad-5 based vectors), herpes viruses (preferably herpes simplex virus based vectors), and parvoviruses (preferably “defective” or non-autonomous parvovirus based vectors, more preferably adeno-associated virus based vectors, most preferably AAV-2 based vectors). See, e.g., Ali et al., “The Use Of DNA Viruses as Vectors for Gene Therapy”, Gene Therapy; 1, pp. 367-84 (1994); U.S. Pat. No. 4,797,368, and U.S. Pat. No. 5,139,941.

The invention is further described in the following examples. The examples should not, in any manner, be understood as limiting the generality of the present specification and claims.

Materials

GCB Activity Assay Buffer:

120 mM phosphate/citrate buffer, pH=5.5, 1 mM EDTA, pH=8.0, 0.25% Triton X-100, 0.25% taurocholate, 4 mM α-mercaptoethanol

pGC-12 Vector

pVL1392 (Pharmingen, USA) with GCB wt cDNA sequence (SEQ ID NO 2) inserted between EcoRV and XbaI.

TABLE 1

Sequence of primers used for cloning the wt GCB coding region and inserting signal peptides
into the pGCBmat plasmid as described in Example 1.

| | | |
|---|---|---|
| SO49 (WT-sp-BglII): | 5′-CGCAG ATCTG ATGGC TGGCA GCCTC ACAGG ATTGC-3′ | (SEQ ID NO:24) |
| SO50 (WT-stop-EcoRI): | 5′-CCGGA ATTCC CATCA CTGGC GACGC CACAG GTAGG TG-3′ | (SEQ ID NO:25) |
| SO51 (WT-mature-SacI): | 5′-ACGCG AGCTC GCCCC TGCAT CCCTA AAAGC TTCGG-3′ | (SEQ ID NO:26) |
| SO52 (SPegt-NheI/SacI-as): | 5′-GCGTT GACGG CAGTC AGAGT TGACA GAAGG GCCAG CCAGC | (SEQ ID NO:27) |
| | AAAGG ATAGT CATG-3′ | |
| SO53 (SPegt-NheI/SacI-s): | 5′-CTAGC ATGAC TATCC TTTGC TGGCT GGCCC TTCTG TCAAC | (SEQ ID NO:28) |
| | TCTGA CTGCC GTCAA CGCAG CT-3′ | |
| SO54 (SPegt-NheI/SacI-as): | 5′-CCTGC TACTG CTCCC AGCAG CAGTG AAAG AGTCC AAAGT | (SEQ ID NO:29) |
| | GGCAG CATG-3′ | |
| SO55 (SPegt-NheI/SacI-s): | 5′-CTAGC ATGCT GCCAC TTTGG ACTCT TTCAC TGCTG CTGGG | (SEQ ID NO:30) |
| | AGCAG TAGCA GGAGC T-3′ | |

TABLE 2

Primers used for introduction of N-glycosylation sites
randomly as described in Example 2 Written with the
nucleotide sequence from 5′ to 3′.

| | | |
|---|---|---|
| SO60: | CAGCTGGCCATGGGTACCCGG | (SEQ ID NO:31) |
| SO90: | CCCTCCAAATCCCTTCACTTTCTGG | (SEQ ID NO:32) |
| SO116: | GAGTTTTTGGTTCTTGCCGGGTCC | (SEQ ID NO:33) |
| SO128: | CCTTCACTGTCTGGTTCTTCTGTTCTGGC | (SEQ ID NO:34) |
| SO130: | CCGTCACGTTCTGGAACTTCTGTTCTGGC | (SEQ ID NO:35) |
| SO131: | CCAAACCAGACCTTCCAGAAAGTGAAGGG | (SEQ ID NO:36) |
| SO132: | CCTTCGTTTTGTTGAACTTCTGTTCTGGC | (SEQ ID NO:37) |
| SO133: | CCAGAAAACAAGACCCAGAAAGTGAAGGG | (SEQ ID NO:38) |
| SO134: | CCGGTTCCGTTTTCAGAGAAGTACGATTTAAG | (SEQ ID NO:39) |
| SO135: | CCAGAACAGAAGTTCCAGAAAGTGAAGGG | (SEQ ID NO:40) |
| SO136: | ATTCCAGTTTCATTGAAGTACGATTTAAG | (SEQ ID NO:41) |
| SO137: | GGTACCTTCAGCCGCTATGAGAGTACACG | (SEQ ID NO:42) |
| SO138: | ATTCCTTCGGTAGAGTTGTACGATTTAAG | (SEQ ID NO:43) |
| SO139: | GGTAACTTCAGCCGCTATGAGAGTACACG | (SEQ ID NO:44) |
| SO140: | ATTCCTTCTTCAGAGAAGTTCGATTTAAG | (SEQ ID NO:45) |
| SO141: | GGTACCAACAGCACCTATGAGAGTACACG | (SEQ ID NO:46) |
| SO142: | GGTGTCTTGTTCTTGGTATCTTCCTCTGG | (SEQ ID NO:47) |
| SO143: | GGTACCTTCAACCGCACCGAGAGTACACG | (SEQ ID NO:48) |
| SO144: | GGTATCTTGGTCTTGTTATCTTCCTCTGG | (SEQ ID NO:49) |
| SO145: | GGTACCTTCAGCAACTATACTAGTACACG | (SEQ ID NO:50) |
| SO146: | GGTATCTTGAGCGTGGTATTTTCCTCTGG | (SEQ ID NO:51) |
| SO147: | GGTACCTTCAGCCGCAATGAGAGTACACG | (SEQ ID NO:52) |
| SO148: | GGTATCTTGAGCTTGGTATCTTCCTCTGG | (SEQ ID NO:53) |
| SO149: | CCAGAGAACGATACCAAGCTCAAGATACC | (SEQ ID NO:54) |
| SO150: | CTGGGTGTAGTTGTCCCCGGGCTGTCCCTTGAGTGACC | (SEQ ID NO:55) |
| SO151: | CCAAACGAAACTACCAAGCTCAAGATACC | (SEQ ID NO:56) |
| SO152: | GTGGGTGATGTTCCCGGGCTCTCCCTTGAGTGACC | (SEQ ID NO:57) |
| SO153: | CCAGAGGAAGATACCAAGCTCAAGATACC | (SEQ ID NO:58) |
| SO154: | GTGGTAGATGTCCCCGGGCTGTCCCTTGAGTGACC | (SEQ ID NO:59) |
| SO155: | GGTCAAACAAGACACAGCCCGGGGACATCTACCAC | (SEQ ID NO:60) |
| SO156: | CTGTCAGCACCGTCTTGTTCCAGTGGGGC | (SEQ ID NO:61) |
| SO157: | GGTCACTCAAGGGACAGCCCGGGGACATCTACCAC | (SEQ ID NO:62) |
| SO158: | CTGTGGTCACGTTCTTTGCCCAGTGGGGC | (SEQ ID NO:63) |
| SO159: | GCCCAACTGGACTAAGGTGGTGCTGACAG | (SEQ ID NO:64) |
| SO160: | CTGTCAGGTTCACCTTTGCCCAGTGGGGC | (SEQ ID NO:65) |
| SO161: | GCCCCACACCGCAACCGTGGTGCTGACAG | (SEQ ID NO:66) |
| SO162: | CTGTCAGCACCACCTTTGCCCAGTGGGGC | (SEQ ID NO:67) |
| SO163: | GCCCCACTGGGCAAAGGTGGTGCTGACAG | (SEQ ID NO:68) |

Cerezyme was kindly provided by Dr. E. Beutler, Scripps Institute, CA, USA.

J774E was kindly provided by G. Grabowski, Cincinnati, Ohio, US

Methods

GCB Activity Assay using PNP-Glucopyranoside or 4-MU-Glucopyranoside Substrate

The enzymatic activity of recombinant GCB is measured using p-nitrophenyl-β-D-glucopyranoside (PNP-Glu) or the fluorescent compound 4-methylumbelliferyl-β-D-glucopyranoside (4-MUGlu) as a substrate. Hydrolysis of the PNP-Glu substrate generates p-nitrophenyl, which can be quantified by measuring absorption at 405 nm using a spectrophotometer, as previously described (Friedmann et al., 1999, Blood 93; 2807-2816). Hydrolysis of the 4MUGlu substrate generates 4-methylumbelliferone, which can be quantified by measuring fluorescence at 460 nm (exitation at 360 nm) using a PolarStar Galaxy spectrofluorometer. The assay is carried out under conditions which partially inhibit non-GCB glucosidase activities, such conditions being achieve by using a phosphate/citrate buffer pH=5.5, 0.25% Triton X-100 and 0.25% taurocholate.

The assay is run in a final volume of 200 μl, containing GCB Activity Assay Buffer and 4 mM PNP-Glu or 3 mM 4-MUGlu. The enzymatic hydrolysis is initiated by adding GCB and the reaction is allowed to proceed for 1 hour at 37° C. before being stopped by adding 50 μl 1 M NaOH and measuring absorption at 405 nm. A reference standard curve of p-nitrophenyl or 4-methylumbelliferone, assayed in parallel, is used to quantify concentrations of GCB in samples to be tested.

In Vitro Uptake and Stability of GCB Polypeptide in Macrophages

The murine monocyte/macrophage cells line, J774E (Mukhopadhyay and Stahl, Arch Biochem Biophys Dec. 1, 1995;324(1):78-84 and Diment et al., J Leukoc Biol November 1987;42(5):485-90) is used to study the uptake and stability of GCB polypeptides. Cells are grown in alpha-MEM (supplemented with 10% fetal calf serum, 1×Pen/Strep, and 60 μM 6-thioguanine), seeded (200,000 cells pr. well) in the above-mentioned media containing 10 μM conditol B epoxide, CBE (an irreversible GCB inhibitor) and incubated for 24 hr at 37° C.

Before starting the uptake assay, cells are washed in 0.5 ml HBSS (Hanks balanced salt solution). The uptake is done in a 200 μl volume, containing the appropriate concentration of GCB polypeptide (a dosis response curve is made with GCB concentrations in the range of 25-400 mU/ml). As a control, yeast mannan (final concentration 1.4 mg/ml) is added to inhibit the uptake through the macrophage mannose receptor. The cells are incubated for 1 hr at 37° C. and washed three times with 0.5 ml cold HBSS.

To measure the amount of GCB taken up by the J774E cells, cells are lyzed in 200 μl GCB Activity Assay Buffer with 4 mM PMP-Glu and incubated for 1 hr at 37° C. Then, the hydrolysis is stopped by addition of 50 μl 1M NaOH and OD405 is measured. The data are analysed by non-linear regression using GraphPad Prizm 2.0 (GraphPad Software, San Diego, Calif.)

To study the stability of GCB polypeptides in J774E cells, CBE treated cells are incubated with 400 mU/ml GCB for 1 hr at 37° C. Then, cells are washed 3 times in HBSS to remove extracellular GCB and incubated in HBSS. A time-course study is done by lyzing the cells after 30 min, 1 hr, 2 hr, 3 hr, 4 hr, and 5 hr in 200 μl GCB Activity Assay Buffer with 4 mM PNP-Glu and incubating the samples for 1 hr at 37° C. before stopping the hydrolysis with 50 μl 1 M NaOH and measuring OD405. The data are analysed by non-linear regression using GraphPad Prizm 2.0 (GraphPad Software, San Diego, Calif.).

SapC Activation of GCB Polypeptides

Phosphatidyl serine from bovine brain is prepared for assay by being dissolved in 1:1 vol methanol:chloroform, drying it down in aliqouts and stored at −20° C. The day of the assay, an aliquot is dissolved and diluted in buffer (120 mM phosphate buffer pH=4.7, 1 mM EDTA, 2 mM β-mercaptoethanol) and sonicated for 10 min. GCB polypeptide activation by SapC was done in a total volume of 200 μl containing 1.25 mU/ml GCB polypeptide, 120 mM phosphate buffer pH=4.7, 1 mM EDTA, 2 mM β-mercaptoethanol, 5 μg/ml phosphatidylserine, 4 mM PNP-Glu and SapC (produced as described in Example 4). The assay is done by pre-incubating GCB polypeptide, lipid and SapC for 20 min at room temperature before starting the assay by addition of the substrate. The reaction mixture is incubated for 1 hr at 37° C. before the hydrolysis is stopped by addition of 50 μl 1 M NaOH and measuring OD405. The data are analysed by non-linear regression using GraphPad Prizm 2.0.

Assays for Determination of Increase In Vivo Activity/Functional In Vivo Half-Life

Increased in vivo activity/functional in vivo half-life is measured using the uptake assays described below. The intracellular activity is measured at different time points after incubation with the GCB polypeptide and the time to which half of the initial activity is present is calculated using standard software programs, e.g. GraphPad Prizm 2.0.

Alternatively, activity in different liver cells after infusion of GCB polypeptide into live animals is determined (Friedman et al. Blood 93, 2807-2816, 1999). Briefly, the GCB polypeptide is infused intravenously into animals. The animals are sacrificed at different time points after the infusion and different liver cell fractions isolated using a combination of Percoll (Sigma) centrifugation and magnet-based isolation of cells with phagocytic capacity. The amount of GCB activity retained in the cells after different time points is determined using the GCB Activity Assay as described above. Furthermore, lysosomes can be isolated from these cells using further Percoll centrifugations and preferably magnetic chromatography in order to measure the lysosomal activity of the GCB polypeptide (Diettrich et Al. FEBS Letts. 1998:441;369-72).

As an example, in vivo uptake of a GCB polypeptide is determined by giving 6-8 week-old Balb/c mice a single bolus injection into the tail vein using 40 units GCB polypeptide per. gram body weight. As a control, mannosylated BSA are used to determine the endogenous level of GCB.

Measurement of serum half-life. For pharmacokinetics studies, tail vein bleeds (˜10 μl/bleed) are done every 10 seconds (up til 5-10 minutes after administration) and sera from these bleeds are assayed for GCB activity using the GCB Activity Assay. Serum concentration-time data are described by first order exponential equations and the serum half-life is calculated from this.

Organ distribution of GCB polypeptide. To determine the organ distribution, animals are killed 20 minutes post-injection. The liver, spleen, heart, lung, brain and kidneys are excised and tissue homogenates are prepared and assayed for GCB activity. The bio-distribution is given as GCB activity recovered per gram wet weight tissue.

Hepatocellular distribution: Mice are administered a single bolus tail vein injection of GCB polypeptide or mannosylated BSA (controls). 20 min, 1 h, 3 h, 8 h, 16 h, 24 h, 36 h, 72 h, and 144 h minutes postinjection, livers of anesthetized mice are perfused in situ with PBS and collagenase D and the different liver populations (parenchymal, kupffer and endothetial cells) are separated as previously described (Friedmann et al., 1999, Blood 93; 2807-2816) or by magnetic cell separation (MACS) using cdl lb microbeads (Miltenyi Biotec Inc.). These separated cell populations are then assayed for GCB activity, using the GCB Activity Assay and the data are given as: 1) GCB activity per gram liver and 2) GCB activity per 10⁶ cells per gram liver.

Isolation of Kupffer Cells

Mice were euthanized and livers perfused in situ via the portal vein, with 0.5 u/ml collagenase solution (Collagenase D No. 108882, Roche Diagnostics) for 4-5 minutes. Liver was then removed and submerged in 3 ml collagenase solution where it was gently minced and the collagenase was allowed to digest the liver tissue for 1 hour at 37° C. on a rocking table.

After 1 hour of digestion the liver solution was gently homogenizing using a 5 ml serological pipette and PBS was added to a total of 10 ml. In order to remove undigested tissue and get a single cell suspension the solution was filtered through gaze and then through a 60□m nylon mesh.

This single-cell-liver solution was centrifuged by 1800 rpm, 10 min, 18° C., supernatant removed and the pellet resuspended in PBS, 0,5% bovine serum albumin (BSA), 2 mM EDTA. For further purification the cell suspension was centrifuged through a 20% icecold Percoll solution (1,031 g/ml) at 1600 rpm, 5 min, 20° C. in a swing-bucket centrifuge without brakes. The resulting upper layer and interface, containing dead cells and debris, was removed. The purified liver cell fraction, consisting of bepatocytes, Kupffer cells and endothelial cells, was on the bottom of the tube. This fraction was washed twice with PBS, 0,5% BSA, 2 mM EDTA and centrifuged by 1600 rpm, 5 min, 20° C.

The Kupffer cell fraction was isolated according to manufacturer's instructions, using an anti-MHC Class II-conjugated magnetic bead over a LS+ MidiMACS separation column (Miltenyi Inc.). Briefly, after the last centrifugation the cell fraction was resuspended in 0.45 ml PBS, 0.5% BSA, 2 mM EDTA followed by addition of anti-MHC Class II-conjugated magnetic beads and the anti-MHC Class II-positive cells where eluted with PBS, 0.5% BSA, 2 mM EDTA. The eluted cell fraction, consisting of Kupffer cells, was finally concentrated by centrifugation 1600 rpm, 5 min, 20° C. and resuspended in a small volume of PBS 0.5% BSA, 2 mM EDTA.

Approximately 1.2×10⁶ Kupffer cells were obtain from one liver. GCB activity was determined by use of the PNP GCB Activity Assay.

Proteolytic Stability

The proteolytic stability of a GCB polypeptide is measured by incubating the polypeptide (e.g. a mutein) and the reference (e.g. wt GCB) with extracts of rat liver lysosomes at pH 4.5 to 5.0. The incubation is run from 1 to 24 hours with samples taken out every 10 to 60 minutes and the left over enzymatic activity is determined using the PNP assay. The proteolytic half-life of wt and mutein is then determined. A method for the preparation of the lysosomal extracts for digestion of proteins is given by Coffey and de Duve, J. Biol. Chem. 243, pp. 3255-3263, 1968.

Site-Directed Mutagenesis

Constructions of site-directed mutations were performed using PCR with oligonucleotides containing the desired amino acid exchanges or additions (e.g. to introduce glycosylation sites). The resulting PCR fragment was cloned into the GCB expression vector using approparite restriction enzymes and subsequently DNA sequenced in order to confirm that the construct contained the desired exchanges.

### EXAMPLES

### Example 1

Production of WT GCB

Cloning and Expression in Insect Cells

A human fibroblast cDNA library was obtained from Clontech (Human fibroblast skin cDNA cloned in lambda-gt11, cat# HL1052b). Lambda DNA was prepared from the library by standard methods and used as a template in a PCR reaction with either SO49 and SO50 as primer (amplifies the GCB coding region with the human signal peptide from the second ATG) or SO50 and SO51 as primer (amplifies the mature part of the GCB coding region) (see Table 1 in the Materials section).

The PCR products were reamplified with the same primers and agarose gel purified. Subsequently the SO49/50 PCR product was digested with BglII and EcoRI and cloned into the pBlueBac 4.5 vector (Invitrogen, Carlsbad, Calif., USA, Carlsbad, Calif., USA) digested with BamHI and EcoRI. Sequencing confirmed that the insert is identical to the wtGCB sequence as given in SEQ ID NO 2. The resulting plasmid was used for infection of insect cells with the GCB being partly secreted from the cells due to the human signal sequence as described in Martin et al., DNA 7, pp. 99-106, 1988. The SO50/51 PCR product was digested with SacI and EcoRI and cloned into the pBlueBac 4.5 vector (Invitrogen, Carlsbad, Calif., USA) digested with the same enzymes resulting in the pGCBmat plasmid. Two different signal sequences were inserted upstream of the mature GCB codons in order to increase the secreted amount of enzyme. The baculovirus ecdysteroid UDPglucosyltransferase (egt) signal sequence (Murphy et al., Protein Expression and Purification 4, 349-357, 1993) was inserted by annealling SO52 and SO53 (Table 1) and the human pancreatic lipase signal sequence (Lowe et al., J. Biol. Chem. 264, 20042, 1989) was inserted by annealling SO54 and SO55 (Table 1) and cloning them into the NheI and SacI digested pGCBmat plasmid. Infection of Spodoptera frugiperda (Sf9) cells of the resulting plasmid was done according to the protocols from Invitrogen, Carlsbad, Calif., USA.

Purification of GCB Polypeptides Produced in Insect Cells

Polypeptides with GCB activity were purified as described in U.S. Pat. No. 5,236,838, with some modifications. Cells were removed from the culture medium by centrifugation (10 min at 4000 rpm in a Sorvall RC5C centrifuge) and the supernatant microfiltrated using a 0.22 μm filter prior to purification. DTT was added to 1 mM and the culture supernatant was ultrafiltrated to approximately 1/10 of the starting volume using a Vivaflow 200 system (Vivascience). The concentrated media was centrifuged to remove possible aggregates before application on a Toyopearl Butyl650C resin (TosoHaas) previously equilibrated in 50 mM sodium citrate, 20% (v/v) ethylene glycol, 1 mM DTT, pH 5.0. This chromatographic step was performed at room temperature. The resin was washed with at least 3 column volumes of 50 mM sodium citrate, 20% (v/v) ethylene glycol, 1 mM DTT, pH 5.0 (until the absorbance at 280 nm reaches baseline level) and GCB was eluted with a linear gradient from 0% to 100% 50 mM sodium citrate, 80% (v/v) ethylene glycol, 1 mM DTT, pH 5.0. Fractions were collected and assayed for GCB activity using the Activity Assay (PNP-Glu). Usually, wt GCB starts to elute at approx. 70% (v/v) ethylene glycol.

The subsequent purification was done by either of the following two methods. #2 method results in GCB of a higher purity.

Method #1

GCB enriched fractions from the first process step were pooled and diluted approx. 4 times with a buffer containing 50 mM sodium citrate, 5 mM DTT, pH 5.0 to reduce the ethylene glycol content to 20% (or lower). In the second HIC purification step the diluted and partially purified GCB was applied on a Toyopearl phenyl resin (TosoHaas) equilibrated in 50 mM sodium citrate, 1 mM DTT, pH 5.0 (Buffer A) before use. After application, the resin was washed with at least 3 column volumes of 50 mM sodium citrate, pH 5 (until the absorbance at 280 nm reaches baseline level) and GCB was then eluted with a linear ethanol gradient from 0% to 100% buffer B (50 mM sodium citrate, 50% (v/v) ethanol, 1 mM DTT, pH 5.0). Highly purified fractions of GCB (wildtype >95% pure), identified using the GCB Activity Assay, start to elute at approx. 40% ethanol. The purified GCB bulk product was dialyzed against 50 mM sodium citrate, 0.2 M mannitol, 0.09% tween80, pH 6.1 to retain the GCB activity upon subsequent storage at 4-8° C. or at −80° C.

Method #2

GCB enriched fractions eluted from the Toyopearl butyl650C resin were pooled and applied at 4° C. on a SP sepharose resin (Amersham Pharmacia Biotech) previously equilibrated in 25 mM sodium citrate, 1 mM DTT, 10% ethylene glycol, pH 5.0. After application, the resin was washed with 25 mM sodium citrate, 1 mM DTT, 10% ethylene glycol, pH 5.0 (until absorption at 280 nm reached baseline level) and GCB was then eluted with a linear gradient from 0 to 100% 0.25 M sodium citrate, 1 mM DTT, 10% ethylene glycol, pH 5.0. GCB begins to elute around 0.15 M sodium citrate. Fractions containing GCB were pooled and applied at room temperature onto a Phenyl sepharose High Performance (Pharmacia Biotech) previously equilibrated in 25 mM sodium citrate 1 mM DTT, pH 5.0. After application, the resin was washed with 25 mM sodium citrate 1 mM DTT, pH 5.0 until absorption at 280 nm reached baseline level, and GCB was then eluted with a linear ethanol gradient from 0 to 100% 25 mM sodium citrate 1 mM DTT 50% ethanol pH 5.0. GCB typically elutes around 35% ethanol.

The purified GCB bulk product was dialyzed against either 50 mM sodium citrate, 1 mM DTT, pH 5.0 or 50 mM sodium citrate, 0.2 M mannitol, 1 mM DTT, pH 6.1 to retain the GCB activity upon subsequent storage. The purified GCB was concentrated and sterilfiltrered before storage at 4-8° C. or at −80° C. Typically, GCB purified by this method is >95% pure.

### Example 2

Random Introduction of Glycosylation Sites in wtGCB

In order to introduce glycosylation sites randomly in specified regions of the GCB cDNA, a primer was made for each glycosylation site to be introduced into the region. A series of PCRs were performed with mixtures of primers, as follows:

Equimolar amounts of the following primer mixtures were used in the PCR:

| | |
|---|---|
| Random1: | SO90 (wt) + 128 + 130 + 132 |
| Random2: | SO131 + 133 + 135 (wt) |
| Random3: | SO142 + 144 + 146 + 148 (wt) |
| Random4: | SO149 + 151 + 153 (wt) |
| Random5: | SO150 + 152 + 154 (wt) (SmaI) |
| Random6: | SO155 + 157 (wt) (SmaI) |
| Random7: | SO156 + 158 + 160 + 162 (wt) |
| Random8: | SO159 + 161 + 163 (wt) |
| RandomA: | SO60 (wt) + 134 + 136 + 138 + 140 |
| RandomB: | SO137 (wt) + 139 + 141 + 143 + 145 + 147 |

The primers are listed in Table 2 in the Materials section.

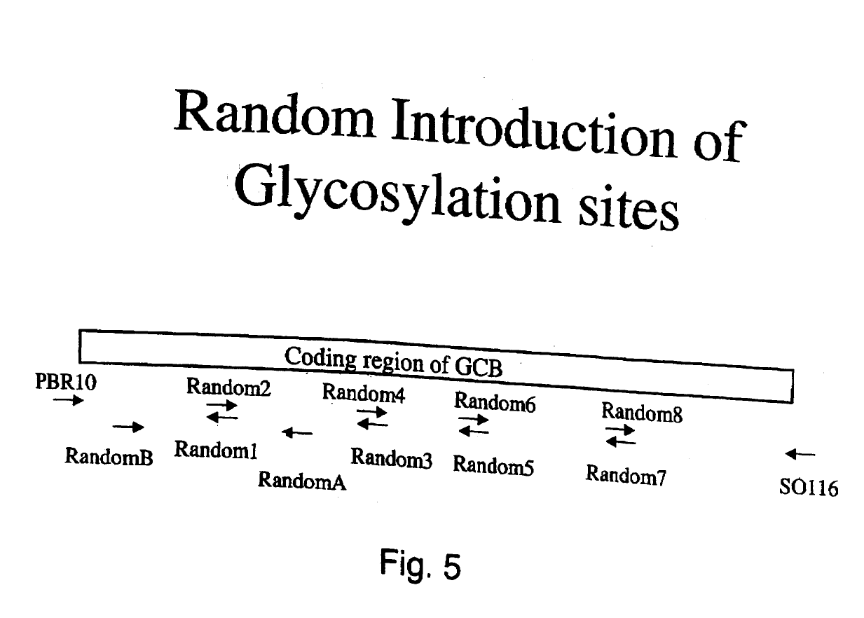

Approximately 100 ng of the wtGCB cDNA is added as template and the PCR is performed under standard conditions. The length of the resulting product is indicated in parenthesis following the primers. FIG. 5 schematically illustrates the relative locations of the primers and PCR spanning the GCB cDNA.

| | |
|---|---|
| PCR1A: Random1 + PBR10 | (390 bp) |
| PCR1B: Random2 + Random3 | (240 bp) |
| PCR1C: RandomA + RandomB | (240 bp) |
| PCR1D: Random4 + Random5 | (165 bp) |
| PCR1E: Random6 + Random7 | (310 bp) |
| PCR1F: Random8 + SO116 | (620 bp) |
| PCR2: SO116 + PBR10 | (1650 bp) |

Products from reactions PCR1A-F were purified from an agarose gel using the Qiagen agarose gel purification kit, and approximately molar amounts were used in a second round of PCR using primers SO116 and PBR10 to reassemble the entire GCB cDNA in a 1650 bp product with a variable number of introduced glycosylation sites. The product from the second PCR was digested with NheI and EcoRI to yield a 1560 bp fragment and directionally cloned into the NheI/EcoRI sites of the pGC-12 vector. The ligation wsa transformed into competent E. coli cells and 1/100 of the transformation was plated onto LB agar containing ampicillin. The remaining 9/10 is grown in LB-Amp overnight and the genomic DNA of the resulting bacteria was isolated and used to produce a plasmid library containing variant GCB cDNAs with different numbers and locations of glycosylation sites.

Plasmid minipreps were then selected at random and sequenced to determine the mutation frequency. If the sequencing revealed a suboptimal level of diversity, the process could be repeated. When a desirable level of diversity was obtained, the plasmid library was transfected into insect cells (Spodoptera frugiperda Sf9 cells) as described in, e.g., protocols published by Invitrogen, Carlsbad, Calif. The resulting transfectants are screened for enzymatic activity using the GCB Activity Assay (PNP). Individual clones are then evaluated, e.g., for enzyme activity and/or cell uptake.

### Example 3

Preparation of GCB with N-Terminal Peptide Additions Using a Site-Directed Mutagenesis Approach

Nucleotide sequences encoding the following N-terminal peptide additions were added to the nucleotide sequence shown in SEQ ID NO 2 encoding wtGCB: (A-4)+(N-3)+(I-2)+(T-1) (representing an extension to the N-terminal of the amino acid sequence shown in SEQ ID NO 1 with the amino acid residues ANIT; SEQ ID NO:69), and (A-7)+(S-6)+(P-5)+(1-4)+(N-3)+(A-2)+(T-1) (ASPINAT; SEQ ID NO:70).

A nucleotide sequence encoding the N-terminal peptide addition (A-4)+(N-3)+(1-2)+(T-1) was prepared by PCR using the following conditions:

PCR 1:

Template: 10 ng pBlueBac5 with wt GCB cDNA sequence

primer SO60 (SEQ ID NO:31): 5′-CAGCT GGCCA TGGGT ACCCG G-3′ and

primer SO85 (SEQ ID NO:71): 5′-TGGGC ATCAG GTGCC AACAT TACAG CCCGC CCCTG CATCC CTAAA AGC-3′

BIO-X-ACT™ DNA polymerase (Bioline, London, U.K.)

1×OptiBuffer™ (Bioline, London, U.K.)

30 cycles of 96° C. 30s, 55° C. 30 s, 72° C. 1 min

PCR 2:

Template: 10 ng pBlueBac5 with wt GCB,

Baculo virus forward primer (SEQ ID NO:72): 5′-TTTAC TGTTT TCGTA ACAGT TTTG-3′ and

primer SO86 (SEQ ID NO:73): 5′-GCAGG GGCGG GCTGT AATGT TGGCA CCTGA TGCCC ACGAC ACTGC CTG-3′

BIO-X-ACT™ DNA polymerase (Bioline, London, U.K.)

1×OptiBuffer™ (Bioline, London, U.K.)

30 cycles of 96° C. 30s, 55° C. 30s, 72° C. 1 min

PCR 3:

3 μl of agarose gel purified PCR1 and PCR2 products (app. 10 ng)

Baculo virus forward primer (SEQ ID NO:72): 5′-TTTAC TGTTT TCGTA ACAGT TTTG-3′

primer SO60 (SEQ ID NO:31): 5′-CAGCT GGCCA TGGGT ACCCG G-3′

BIO-X-ACT™ DNA polymerase (Bioline, London, U.K.)

1×OptiBuffer™ (Bioline, London, U.K.)

30 cycles of 96° C. 30s, 55° C. 30s, 72° C. 1 min

PCR 3 was agarose gel purified and digested with NheI and NcoI and cloned into pBluebac4.5+wtGCB digested with NheI and NcoI.

After confirmation of the correct mutations by DNA sequencing the plasmid was transfected into insect cells using the Bac-N-Blue™ transfection kit from Invitrogen, Carlsbad, Calif., USA. Expression of the muteins was tested by western blotting and by activity measurement of the muteins using the GCB Activity Assay.

Enzymatic activity in the PNP assay of wtGCB (SEQ ID NO 1) expressed in the expression vector pVLI 392 in insect cells (Sf9) using an analogous method to that described in Example 1 gave 13 units/L, while the N-terminal peptide addition ASPINAT (SEQ ID NO:70) gave 28.5 units/L.

Construction of Libraries of GCB with N-Terminal Peptide Addition

Using random mutagenesis two different libraries were constructed on the basis of GCB polypeptides with an N-terminal extension—library A with an N-terminal extension encoding the following amino acid sequence AXNXTXNXTXNXT (SEQ ID NO:74), and library B with an N-terminal extension encoding ANXTNXTNXT (SEQ ID NO:75).

Primers for library A were designed:

| | |
|---|---|
| SO167: 5′-GTGTC GTGGG CATCA GGTGC CNN(G/C)A | (SEQ ID NO:76) |
| A(C/T)(T/A/G)N(G/C) AC(A/T/C)(T/A/G)N (G/C)AA(C/T) (T/A/G)N(G/C)AC | |
| (A/T/C)(T/A/G)N(G/C)A A(C/T)(T/A/G)N(G/C) AC(A/T/C)GC CCGCC CCTGC ATCCC | |
| TAAAA GC | |
| SO168: 5′-GGCAC CTGAT GCCCA CGACA CTGCC TG | (SEQ ID NO:77) |

Primers for library B were designed using trinucleotides in the random positions.

X is a mixture of trinucleotide codons for all natural amino acid residues, except proline. The trinucleotide codons used were the same as described by Kayushin et al., Nucleic Acids Research, 24, 3748-3755, 1996.

| | |
|---|---|
| SO165: 5′-CGTGG GCATC AGGTG CCAAC (X)AC (A/T/C)AA(C/T) | (SEQ ID NO:78) |
| (X)AC (A/T/C)AA(C/T) (X)AC (A/T/C)GCCC GCCCC TGCAT CCCTA AAAGC | |
| SO166: 5′-GTTGG CACCT GATGC CCACG ACACT GCCTG | (SEQ ID NO:79) |

For both libraries:

| | |
|---|---|
| SO60: | |
| 5′-CAGCT GGCCA TGGGT ACCCG G | (SEQ ID NO:31) |
| pBR10: | |
| 5′-TTT ACT GTT TTC GTA ACA GTT TTG | (SEQ ID NO:72) |

In all PCR reactions BIO-X-ACT™ DNA polymerase (Bioline, London, U.K.) and 1*Optibuffer™ (Bioline, London, U.K.) were used. The PCR conditions were 30 cycles of 94° C. 30 s, 55° C. 1 min, and 72° C. 1 min.

Templates and primers used for preparing a nucleotide sequence encoding the N-terminal extension by the above PCR were as follows:

PCR 1A:

Template: pGC12

Primers: SO60+SO167

PCR 1B:

Template: pGC12

Primers: SO60+SO165

PCR 2A:

Template: pGC12

Primers: SO168+pBR10

PCR 2B:

Template: pGC12

Primers: SO166+pBR10

PCR 3A:

Template: 1 μl of agarose gel purified PCR 1A and 2A products

Primers: SO60+pBR10

PCR 3B:

Template: 1 μl of agarose gel purified PCR 1B and 2B products

Primers: SO60+pBR10

PCR 3A and 3B were agarose gel purified and digested with NheI and NcoI and ligated into pGC-12 digested with NheI and NcoI. The ligation mixture is transformed into competent E. coli as described in Example 2. The diversity of the library was examined by DNA sequencing of different E. coli clones and gave rise to the following amino acid sequences:

Library A:

| | | |
|---|---|---|
| 1: | AFNXTLNKTWN(F/L)T | (SEQ ID NO:80) |
| 2: | TMNNTWNWTWNWT | (SEQ ID NO:81) |
| 3: | -EXTwt | |
| 4: | ALNSTGNLTVDGT | (SEQ ID NO:82) |
| 5: | ASNSTFNLTENLT | (SEQ ID NO:83) |
| 6: | TRNVTINCTUNST | (SEQ ID NO:84) |
| 7: | -EXTwt | |
| 8: | ALNWTYNGTKNVT | (SEQ ID NO:85) |
| 9: | AANWTVNFTGNFT | (SEQ ID NO:86) |
| 10: | -EXT wt | |
| 11: | AXNXTVNSTUNVT | (SEQ ID NO:87) |
| 12: | ANNFTFNGTLNLT | (SEQ ID NO:88) |
| 13: | AGNWTANVTVNVT | (SEQ ID NO:89) |
| 14: | AGNSTSNVTGNWT | (SEQ ID NO:90) |
| 15: | AVNSTMNIHAIPP | (SEQ ID NO:91) |
| | (1 deletion-nonsens) | |
| 16: | AGNGTVNGTINGT | (SEQ ID NO:92) |
| 17: | AVNSTGNXTGNWT | (SEQ ID NO:93) |
| 18: | AGNGTUNGTSNLT | (SEQ ID NO:94) |
| 19: | -EXT wt | |
| 20: | AMNSTKNSTLNIT | (SEQ ID NO:95) |
| 21: | AFNYTSKNST | (SEQ ID NO:96) |
| 22: | -EXT wt | |
| 23: | AVNATMNWTANGT | (SEQ ID NO:97) |
| 24: | ASNSTNNGTLNAT | (SEQ ID NO:98) |
| 25: | ARNKTKNFTINLT | (SEQ ID NO 99) |
| 26: | APNITUNDTVNMT | (SEQ ID NO:100) |
| 27: | AQNKTFNFTMNCT | (SEQ ID NO:101) |
| 28: | ALNVTWNCTLNLT | (SEQ ID NO:102) |
| 29: | ALNTTWTNLT | (SEQ ID NO:103) |

Library B:

| | | |
|---|---|---|
| 1: | ANTTNFTNET | (SEQ ID NO:104) |
| 2: | ANWTNRTNCT | (SEQ ID NO:105) |
| 3: | ANWTNFTNWT | (SEQ ID NO:106) |
| 4: | PTGLIGTNFT | (SEQ ID NO:107) |
| 5: | ANWTNKTNFT | (SEQ ID NO:108) |
| 6: | ANNTNLTNAT | (SEQ ID NO:109) |
| 7: | ANYTNWTNFT | (SEQ ID NO:110) |
| 8: | ANTTNQTNDT | (SEQ ID NO:111) |
| 9: | -EXT wt | |
| 10: | ANRTNWTNTT | (SEQ ID NO:112) |
| 11: | PTATNHTNST | (SEQ ID NO:113) |
| 12: | -EXT wt | |
| 13: | ANWTNQTNQT | (SEQ ID NO:114) |
| 14: | ANWTNWTNAT | (SEQ ID NO:115) |
| 15: | ANFTNKTNMT | (SEQ ID NO:116) |
| 16: | ANHTNETNAT | (SEQ ID NO:117) |
| 17: | AN(C/W)TNFTNET | (SEQ ID NO:118) |
| 18: | ANLDKIHKUH | (SEQ ID NO:119) |
| | (insertion-nonsens) | (SEQ ID NO:110) |
| 19: | ANCFTNQTNFT | (SEQ ID NO:111) |
| 20: | ANWTNWTNEWT | (SEQ ID NO:112) |
| 21: | ANCTNWTNCT | |
| 22: | -EXT wt | |
| 23: | -EXT wt | |
| 24: | CHPYNWTNWT | (SEQ ID NO:113) |
| 25: | ANETNYTNET | (SEQ ID NO:114) |
| 26: | ANWTNWT | (SEQ ID NO:115) |
| 27: | AKPYKSYKFY (SEQ ID NO:116) | |
| | (insertion-nonsens) | |
| 28: | ANITNKITNWT | (SEQ ID NO:117) |
| 29: | ANWTNMTNIT | (SEQ ID NO:118) |
| 30: | ANNTNRTNFT | (SEQ ID NO:119) |
| 31: | ANWTNWTNWT | (SEQ ID NO:120) |
| 32: | ANWRTNHTNKT | (SEQ ID NO:121) |
| 33: | -EXT wt | |
| 34: | ANQTNITNWT | (SEQ ID NO:122) |

Library B was transfected into insect cells using the Bac-N-Blue™ transfection kit from Invitrogen, Carlsbad, Calif., USA. First, 96 plaques from Library B were picked and tested by activity measurement (PNP GCB Activity Assay). Plaques were selected as follows: 3 with high activity, 3 with medium activity and 3 with low or no activity, and virus was purified for DNA sequencing resulting in the following amino acid sequences:

High activity:

1-1: Mixed sequence

1-2 (SEQ ID NO:133): ANFTNVATNQT

1-3 (SEQ ID NO: 134): (A)(N)TTXLTN(K)T

Medium activity:

2-1 (SEQ ID NO:135): ANKTN(S/C)TNIT

2-2: Mixed sequence

2-3 (SEQ ID NO:136): ANWTNCTN(I)T

Low activity:

3-1 (SEQ ID NO:137): ANWTN(F/L)TNWT

3-2 (SEQ ID NO:138): CQLDURSTNET

3-3: No sequence

From both libraries 96 plaques were picked and tested by activity measurement (PNP GCB Activity Assay). From each library 6 plaques with high activity were selected and virus were purified for DNA sequencing. The amino acid sequence encoded by the different clones were:

Library A:

| | |
|---|---|
| 1: Mixed sequence | |
| 2: Mixed sequence | |
| 3: Mixed sequence | |
| 4: WT | |
| 5: ANNTNYTNWT | (SEQ ID NO:139) |
| 6: ANNTNYTNWT | (SEQ ID NO:140) |

Library B:

| | |
|---|---|
| 1: AANDTUNWTVNCT | (SEQ ID NO:141) |
| 2: ATNITLNYTANTT | (SEQ ID NO:142) |
| 3: WT | |
| 4: AANSTGNITINGT | (SEQ ID NO:143) |
| 5: AVNWTSNDTSNST | (SEQ ID NO:144) |

The activity of the positives after plaque purification are shown in Table X in Example 6 below.

### Example 4

Production of SAPC

Expression of a Synthetic Sap C Gene in E. coli

A plasmid expression vector for expression of Saposin C with a His-tag was kindly obtained from Dr. Gregory A. Grabowski, Cincinnati, Ohio. The plasmid is described in Qi et al. J. Biol. Chem. 269, 16746-16753, 1994, and the expression of it in the E. coli strain BL21 (DE3) was performed as described in the same paper.

Purification

Cell pellets from E. coli expressing recombinant Saposin C were solubilized in binding buffer (10 mM Tris, 0.5 M NaCl, 20 mM Imidazol, pH 7.9) containing one tablet of “Complete” protease inhibitor Cocktail (Roche) per 50 ml, and sonicated on ice on a U200S sonicator (IKA) at 80% amplitude for 4 times 20 seconds. The sonicate was centrifuged in a Sorvall RC5C centrifuge with a SS34 rotor at 12000 rpm for 15 minutes at 4° C. The supernatant was filtered through a 0.45 μm filter and applied onto a Ni-loaded HiTrap™ Chelating column (Pharmacia) previously equilibrated in binding buffer. The resin was washed with binding buffer until the absorption at 280 nm reached baseline levels, and bound protein was eluted using a linear gradient from 0-100% B buffer (10 mM Tris, 0.5 M NaCl, 0.5 M Imidazol pH 7.9). Fractions enriched in Saposin C were pooled and ammonium sulfate was added to 0.75 M before application onto a Toyopearl Butyl 650S resin previously equilibrated in 10 mM Tris pH 7.9, 0.75 M ammonium sulfate. After application, the resin was washed in 10 mM Tris pH 7.9, 0.75 M ammonium sulfate until absorption at 280 nm reached baseline levels. Bound protein was eluted using a linear gradient from 0-100% B (10 mM Tris pH 7.9 Saposin C, eluting around 0.10 M ammonium sulfate, was pooled and the buffer was exchanged on a Vivaspin20 (Vivascience) to 50 mM sodium Citrate pH 5.8. The protein sample was sterile-filtered before storage at −80° C.

### Example 5

Construction of a Saposin C-GCB Fusion Polypeptide

Fusion polypeptides of wtSaposin C (SEQ ID NO 3) and wtGCB (SEQ ID NO 1, wherein X is R) were constructed using standard cloning methods known in the art by making one nucleotide sequence expressing either of the following polypeptides:

SaposinC-linkerpeptide1-GCB or GCB-linkerpeptide2-SaposinC

The composition of specific fusion polypeptides (pGC-53, pGC-54, pGC-64, pGC-65 and pGC-73) are given in table 3 in Example 6.

An example of the amino acid sequence of the fusion polypeptide of the type SaposinC-linkerpeptide-GCB is shown as SEQ ID NO 4.

### Example 6

Properties of GCB Polypeptides of the Invention

GCB polypeptides of the invention were tested for various properties, including GCB activity, stability in J774E cells and uptake in J774E cells. Unless otherwise stated the properties were tested by use of the methods described in the Methods section herein.

In table 3 below the GCB activity of various GCB polypeptides of the invention is listed.

| | | | | Activity after |
|---|---|---|---|---|
| | | | # Glycosylation | Plaque Isolation |
| Plasmid | Vector | Mutations | sites introduced | (U/L) |
| pGC-1 | PBlueBac | 4.5Wt | 0 | 6 |
| pGC-2 | pBlueBac | 4.5K194N | 1 | 16 |
| pGC-3 | pBlueBac | 4.5K194T | 1 | 6 |
| pGC-4 | pBlueBac | 4.5K224N, Q226T | 1 | 4 |
| pGC-5 | pBlueBac | 4.5K293N, V295T | 1 | No plaques |
| pGC-6 | pBlueBac | 4.5N-termANIT (SEQ ID NO: 69) | 1 | 3 |
| pGC-7 | pBlueBac | 4.5E41N | 1 | 2 |
| pGC-8 | pVL1392 | K74N, Q76T | 1 | 31 |
| pGC-9 | pVL1392 | A84N | 1 | 0.05 |
| pGC-10 | pBlueBac | 4.5 K321N | 1 | No plaques |
| pGC-12 | pVL1392 | Wt | 0 | 13 |
| pGC-13 | pVL1392 | N-termASPINAT (SEQ ID NO: 70) | 1 | 29 |
| pGC-14 | pVL1392 | K7N, *9T | 1 | 0.2 |
| pGC-15 | pVL1392 | K106, Y108T | 1 | 0.2 |
| pGC-16 | pVL1392 | K194N, Q200T | 1 | 0.4 |
| pGC-17 | pVL1392 | H206N | 1 | 0.3 |
| pGC-18 | pVL1392 | E222N, K224T | 1 | 6 |
| pGC-19 | pVL1392 | K303N, V305T | 1 | 1.5 |
| pGC-21 | pVL1392 | K293N, V295T | 1 | 29 |
| pGC-22 | pVL1392 | K321N | 1 | 24 |
| pGC-27 | pVL1392 | T132N | 1 | 9 |
| pGC-28 | pVL1392 | I130N | 1 | 7 |
| pGC-36 | pVL1392 | N-term: ASPINATSPINAT (SEQ ID NO: 145) | 2 | 16 |
| pGC-37 | pVL1392 | K194N, K321N | 2 | 13 |
| pGC-38 | pVL1392 | N-term: ASPINAT, K194N, K321N | 3 | 16 |
| pGC-39 | pVL1392 | T132N, K293N, V295T | 2 | 3 |
| pGC-40 | pVL1392 | N-term: ASPINAT, T132N, K293N, V295T | 3 | 3.5 |
| | | N-term: ASPINAT, K194N, E222N, K224T, | | |
| pGC-45 | pVL1392 | K321N | 4 | 13 |
| pGC-47 | pVL1392 | N-term: AGNGTVNGTINGT (SEQ ID NO: 92) | 3 | |
| pGC-48 | pVL1392 | N-term: ASNSTNNGTLNAT (SEQ ID NO: 98) | 3 | |
| pGC-52 | pVL1392 | R495H | | |
| pGC-53 | pVL1392 | Saposin C-(GGGGS)₃ linker-GCB (SEQ ID: 4) | | |
| pGC-54 | pVL1392 | GCB-GGGG linker-Saposin C | | 27 |
| pGC-56 | pVL1392 | N-term: ASPINATSPINAT, K194N, K321N | 4 | |
| pGC-57 | pVL1392 | N-term: ASPINAT, T132N, K194N, K321N | 4 | |
| pGC-58 | pVL1392 | N-term: ASPINAT, T132N, K194N | 3 | |
| pGC-60 | pVL1392 | N-term:ANNTNYTNWT (SEQ ID NO: 140) | 3 | P2: 14 |
| pGC-61 | pVL1392 | N-term: ATNITLNYTANTT (SEQ ID NO: 142) | 3 | P2: 38 |
| pGC-62 | pVL1392 | N-term: AANSTGNITINGT (SEQ ID NO: 143) | 3 | P2: 35 |
| pGC-63 | pVL1392 | N-term: AVNWTSNDTSNST (SEQ ID NO: 144) | 3 | P2: 66 |
| pGC-64 | pVL1392 | GCB-(GGGGS)₃ linker-Saposin C | | 67 |
| pGC-65 | pVL1392 | GCB-GNAT linker-Saposin C | | 54 |
| pGC-66 | pVL1392 | Q166N, A168T | 1 | 79 |
| pGC-67 | pVL1392 | D218N, Y220T | 1 | |
| pGC-68 | pVL1392 | AN N-term extension + R2T | 1 | 37 |
| pGC-69 | pVL1392 | K77N, K79T | 1 | 17 |
| pGC-70 | pVL1392 | T132N, K194N, K293N, V295T, K321N | 4 | |
| pGC-71 | pVL1392 | N-term: ASPINAT, T132N, K194N, K293N, V295T, K321N | 5 | |
| pGC-72 | pVL1392 | P28N, P29L | 1 | 13 |
| pGC-73 | pVL1392 | GCB-Sap C (no linker) | | 16 |

Table 3: The plasmid column shows the number of the GCB polypeptide. The vector column shows the plasmid vector used for expression of the polypeptide. The mutation column shows the amino acid exchanges of the GCB polypeptide. N-terminal extentions are described as N-term followed by the amino acid residues that makes up the extension. Constructs for expression of fusion proteins of Saposin C and GCB are described in the order that they are fused and the amino acid residues making up the linker linking the two polypeptides together. The Activity column gives the units per liter of GCB activity measured by the GCB Activity Assay (PNP-Glu) on the supernatant from Sf9 insect cells infected with one single plaque and grown in 3 ml of media in a 6-well plate. Those labelled with P2 are activity measured of supernatant from virus infection cells grown in 15 ml T75 flasks.

| | Vₘₐₓ | | | K_{M} | | |
|---|---|---|---|---|---|---|
| X Labels | Y | SD | N | Y | SD | N |
| WT | 0.572 | 0.101 | 3 | 87.680 | 23.211 | 3 |
| Cerezyme | 0.518 | 0.144 | 2 | 91.915 | 2.666 | 2 |
| pGC36 | 0.599 | 0.010 | 2 | 70.590 | 22.557 | 2 |
| pGC37 | 0.449 | 0.000 | 1 | 36.300 | 0.000 | 1 |
| pGC38 | 0.478 | 0.000 | 1 | 43.980 | 0.000 | 1 |
| pGC45 | 0.371 | 0.000 | 1 | 27.520 | 0.000 | 1 |
| pGC54 | 0.871 | 0.139 | 3 | 79.073 | 6.450 | 3 |
| pGC56 | 0.392 | 0.000 | 1 | 32.170 | 0.000 | 1 |
| pGC59 | 0.362 | 0.000 | 1 | 30.900 | 0.000 | 1 |
| pGC60 | 0.566 | 0.156 | 2 | 79.133 | 14.030 | 3 |
| pGC61 | 0.738 | 0.105 | 2 | 100.510 | 16.674 | 2 |
| pGC62 | 0.860 | 0.000 | 1 | 110.800 | 0.000 | 1 |
| pGC63 | 0.513 | 0.100 | 2 | 83.105 | 6.456 | 2 |

Table 4: Calculated Vmax and KM for the different GCB polypeptides. Vmax and KM was calculated from dosis-response curves (see FIG. 1).

The uptake and stability of selected GCB polypeptides are shown in FIGS. 1 and 2, respectively.

For the dosis response curves (FIG. 1), a Vₘₐₓ and a K_{M} for uptake was calculated for each of the selected GCB polypeptides (see table 4, wherein Y is the actual value, SD the standard deviation and N the number of assays). As can be seen from table 4, an increase in Vₘₐₓ was observed for the fusion protein (pGC54) and for the N-terminally extended GCB polypeptides (pGC60, pGC61, pGC62, and pGC63) while the KM was unchanged.

Furthermore, the muteins were also tested for their stability in J774E cells (FIG. 2) and a half-life was calculated to be between 50 and 100 sec.

Activation of the different GCB polypeptides by phosphatidyl serine from bovine brain was also tested and a KD was calculated. As can be seen in FIG. 3, the GCB-saposin C fusion protein (pGC54) was far more active compared to Cerezyme and the WT GCB polypetide (a 6.8 and 5.2 fold change in KD, respectively).

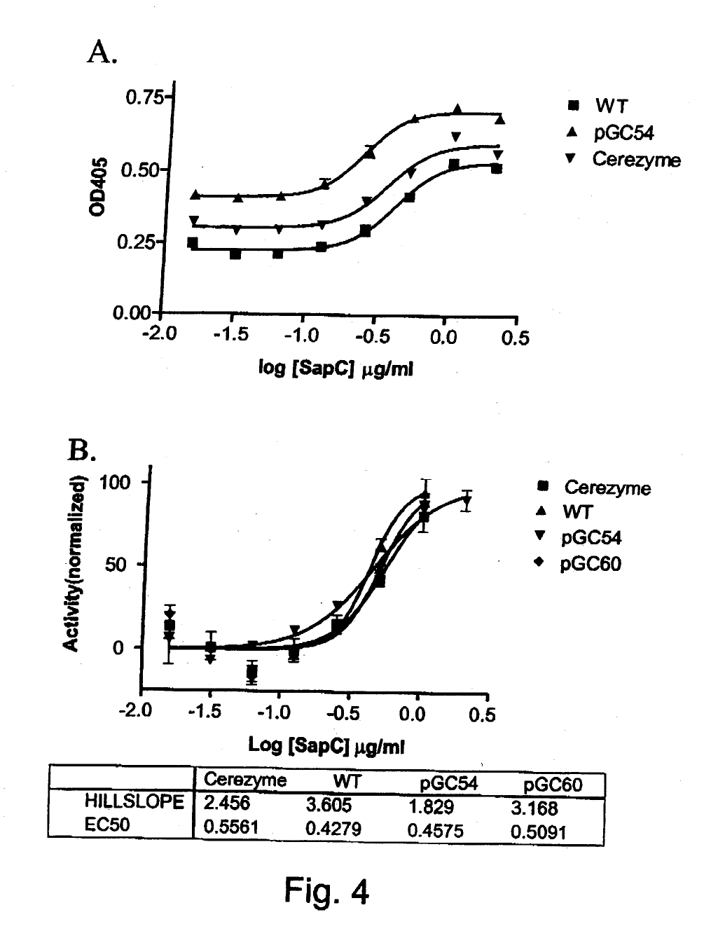

Also, the ability of saposin C to activate a set amount of the different GCB polypeptides was also tested in the presence of 5 μg/ml phosphatidyl serine. As can be seen in FIG. 4A, the basal activity of the fusion protein (pGC54) was higher compared to the WT polypeptide and Cerezyme.

### Example 7

PEGylation of GCB Polypeptides

GCB polypeptides were PEGylated using activated PEG-succinimidyl propionate (SPA-PEG) (Shearwater) in a buffer containing 0.1 M sodium phosphate pH 7.0. PEG was present in 5-120-fold molar excess with respect to the lysines, and protein concentration was 0.8-1.3 mg/ml. The reaction was carried out in 50-120 μl batches at room temperature for 1 hour with agitation, and quenched using a 20-fold excess of glycine. Following the conjugation reaction, excess glycine and PEG were removed by dialysis.

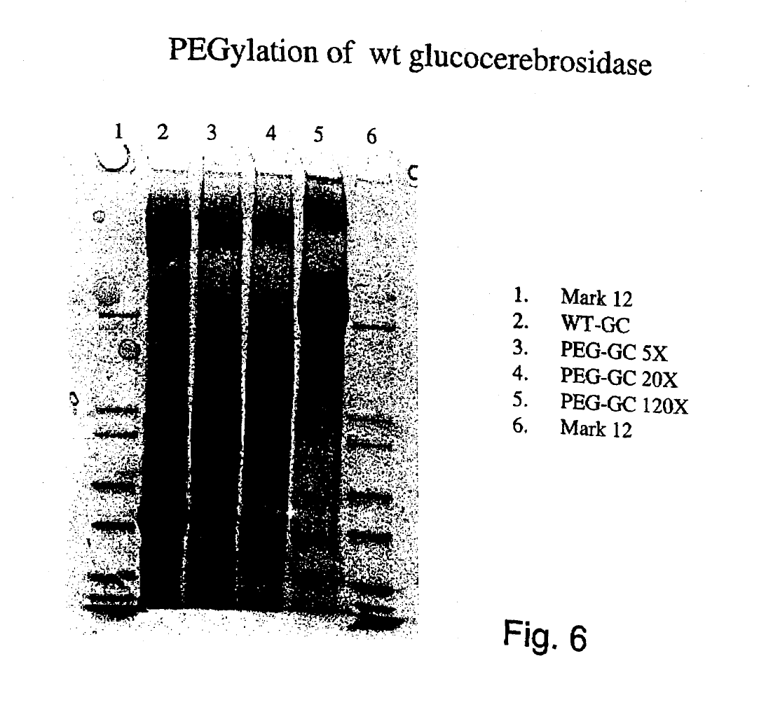

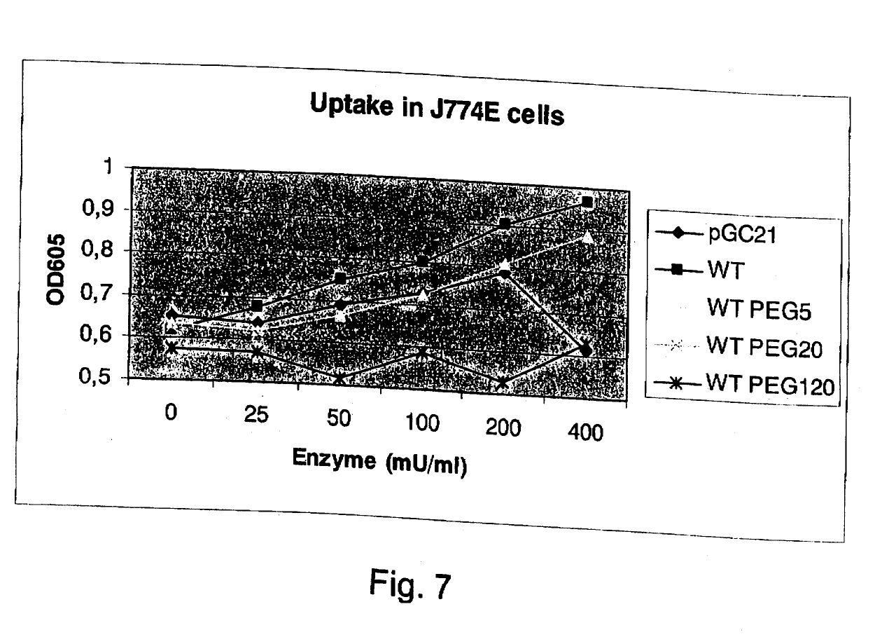

Using the above method rGCB was conjugated with activated SPA-PEG (Mw 5000 Da). rGCB in 50 mM sodium citrate, 0.2 M mannitol, 0.09% tween80, pH 6.1 was dialyzed with 0.1 M sodium phosphate buffer solution, pH 7.0, using a Vivaspin 500 (Vivascience) resulting in a final GCB concentration of 1.7 mg/ml. 25 μl SPA-PEG was solubilized in 0.1 M sodium phosphate buffer solution pH 7.0 to a concentration of 88 mg/ml and immediately added to an equal volume of the enzyme solution, giving a 20 fold excess of PEG with. respect to lysines. The reaction was incubated at room temperature for 1 hour with agitation. The reaction was quenched by adding 20 fold molar excess of glycine. The modification was checked by SDS PAGE and the enzyme activity was measured by using the artificial substrate PNP-glucopyranoside. SDS PAGE showed a number of discrete bands each representing a pegylated GCB species. The major bands corresponded to a GCB molecule with 6-8 conjugated PEG molecules. (FIG. 6). The activity assays revealed that approximately 80% of the GCB activity was retained. The uptake of PEGylated GCB polypeptides was assayed using the J774E in vivo uptake assay. The result is shown in FIG. 7. It is evident that when 1-4 PEG molecules are attached to GCB, uptake is comparable to wildtype.

### Example 8

N-Glycan Structures in WTGCB Expressed in Insect Cells

Approximately 350 μg of purified wtGCB expressed in Sf9 cells were dried in a SpeedVac concentrator, dissolved in 400 μl 6 M guanidinium, 0.3 M Tris-HCl, pH 8.3 and denatured overnight at 37° C. Following denaturation, the disulfide bonds in the protein were reduced by addition of 50 μl 0.1 M DTT in 6 M guanidinium, 0.3 M Tris-HCl, pH 8.3. After 2 h of incubation at ambient temperature the thiol-groups present were alkylated by addition of 50 μl 0.6 M iodoacetamid in 6 M guanidinium, 0.3 M Tris-HCl, pH 8.3. Alkylation took place for 30 min at ambient temperature before the reduced and alkylated protein was buffer changed into 50 mM NH₄HCO₃ using a NAP5 column. The volume of the sample was reduced to approximately 200 μl in a SpeedVac concentrator before addition of 10 μg trypsin. Trypsin degradation was carried out for 16 h at 37° C. The resulting peptides were separated by reversed phase HPLC employing a Phenomenex Jupiter C₁₈ column (0.2*5 cm) eluted with a linear gradient of acetonitrile in 0.1% aqueous TFA. The collected fractions were analysed by MALDI-TOF mass spectrometry before re-purification. Subsequently selected peptides were subjected to N-terminal amino acid sequence analysis.

445 amino acid residues out of 497 (90%) were verified in the GCB sequence either through direct identification using chemical sequencing or through indirect mass identification of peptides using MALDI-TOF mass spectrometry. This is summarised in Table 5.

TABLE 5

| | ⋄ | |
|---|---|---|
| 1 | [symbol] [symbol] [symbol] CDSFDPPT FPALGTFSRY ESTRSGR [symbol] | 50 |
| | ⋄ | |
| 51 | [symbol] [symbol] [symbol] FQKVK [symbol] [symbol] [symbol] | 100 |
| | ⋄ | |
| 101 | [symbol] LLLK [symbol] [symbol] [symbol] [symbol] [symbol] | 150 |
| 151 | EEDTK [symbol] [symbol] [symbol] [symbol] TNGA VNGKGSLK [symbol] | 200 |
| 201 | [symbol] [symbol] YFVK [symbol] [symbol] [symbol] [symbol] | 250 |
| | ⋄ | |
| 251 | [symbol] DFI AR [symbol] [symbol] [symbol] [symbol] | 300 |
| 301 | [symbol] [symbol] [symbol] [symbol] [symbol] | 350 |
| 351 | [symbol] [symbol] [symbol] [symbol] [symbol] | 400 |
| 401 | [symbol] DT FYK [symbol] [symbol] FIPEG SQRVGLVASQ K [symbol] | 450 |
| | ⇓ | |
| 451 | [symbol] [symbol] SSK [symbol] [symbol] [symbol] [symbol] WRRQ 497 | |

The amino acid sequence of wtGCB (SEQ ID NO: 1). Amino acid residues shown in italics are verified through mass identification of a peptide while amino acid residues in bold italics are verified through chemical sequence determination. ⋄ designates the four used N-glycosylation sites while [symbol] designates the potential N-glycosylation site that is not used.

The amino acid sequence of GCB contains five potential N-glycosylation sites at Asn19, Asn59, Asnl46, Asn270, and Asn462. The N-glycosylation site at Asn462 is not used in GCB expressed in CHO cells. Four glycosylated peptides were identified using combined data from MALDI-TOF mass spectrometry and N-terminal amino acid sequencing and purified. Each of these four peptides contains a single N-glycosylation site at Asn19, Asn59, Asn146, and Asn270, respectively. The peptide containing the potential N-glycosylation site at Asn462 was purified and the combined data from MALDI-TOF mass spectrometry and N-terminal amino acid sequencing showed Asn462 to be unoccupied in GCB expressed in Sf9 cells as in CHO cells.

For the peptide containing Asn19 (amino acid residues 8-39) the theoretical mass—including the three S-carboxamido-groups on Cys-residues 4, 16, and 18—is 3608.57 Da. The peptide containing Asn 19-identified through N-terminal amino acid sequence determination—gave experimental masses of 4501.97 Da and 4341.11 Da in MALDI-TOF mass spectrometry. The mass differences between the theoretical mass and the experimental masses are thus 893.40 Da and 732.54 Da. The mass differences correspond to Man₃GlcNAc₂ (892.31 Da) and Man₂GlcNAc₂ (730.26 Da) carbohydrate structures.

Analogously, the peptides containing Asn59 (amino acids 48-74), Asn146 (amino acids 132-155), and Asn270 (amino acids 263-277) were analysed and the attached carbohydrate structures suggested. The results are summarised in Table 6.

TABLE 6
Summary of MALDI-TOF mass spectrometry of the glycosylated wtGCB
peptides. The masses given for the peptide comprising amino acid residues
8-39 includes the mass of the S-carboxamido-groups on Cys-residues 4, 16, and 18.

| Amino | Theoretical | | | |
|---|---|---|---|---|
| acid | peptide | Experimental | Mass | Suggested carbohydrate |
| residue no. | mass | masses | differences | structures and their masses |
| 8-39 | 3608.57 Da | 4501.97 Da | 893.40 Da | Man₃GlcNAc₂; 892.31 Da |
| | | 4341.11 Da | 732.54 Da | Man₂GlcNAc₂; 730.26 Da |
| 48-74 | 2962.54 Da | 4001.24 Da | 1038.70 Da | Man₃GlcNAc₂Fuc; 1038.38 Da |
| | | 3855.97 Da | 893.43 Da | Man₃GlcNAc₂; 892.31 Da |
| 132-155 | 2846.26 Da | 3887.95 Da | 1041.69 Da | Man₃GlcNAc₂Fuc; 1038.38 Da |
| | | 3740.16 Da | 893.90 Da | Man₃GlcNAc₂; 892.31 Da |
| 263-277 | 1630.82 Da | 2666.85 Da | 1036.03 Da | Man₃GlcNAc₂Fuc; 1038.38 Da |
| | | 2504.73 Da | 873.91 Da | Man₂GlcNAc₂Fuc; 876.33 Da |

The different carbohydrate structures were further characterised by subjecting the four peptides carrying carbohydrate to sequential exo-glycosidase treatments in combination with mass determinations.

Below the typical N-glycan structure found on glycoproteins expressed in Sf9 cells is shown. The fucose-residue (Fuc) linkage is normally α1,6, but can also be α1,3 (indicated by “?”)

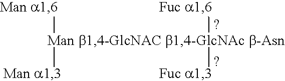

The sequential exo-glycosidase treatments consisted of overnight incubations at 37° C. with the following enzymes—α(1-2,3,4)mannosidase, β(1-4)mannosidase, a(1-6)fucosidase, and N-glycosidase A. Between each enzyme treatment the mass of the peptides was determined using MALDI-TOF mass spectrometry.

Following the treatments with α(1-2,3,4)mannosidase and β(1-4)mannosidase it was still possible to obtain reasonable mass spectra of the peptides. However, the treatment with α(1-6)fucosidase introduced a significant amount of low molecular mass contaminants in the peptide samples and it was only possible to obtain data for the carbohydrate structure on Asn270. The same problem was also observed for the subsequent treatment with N-glycosidase A.

The results are summarised in Table 7.

In general, the results obtained are in accordance with the glycostructure shown above with the following specific positional details.

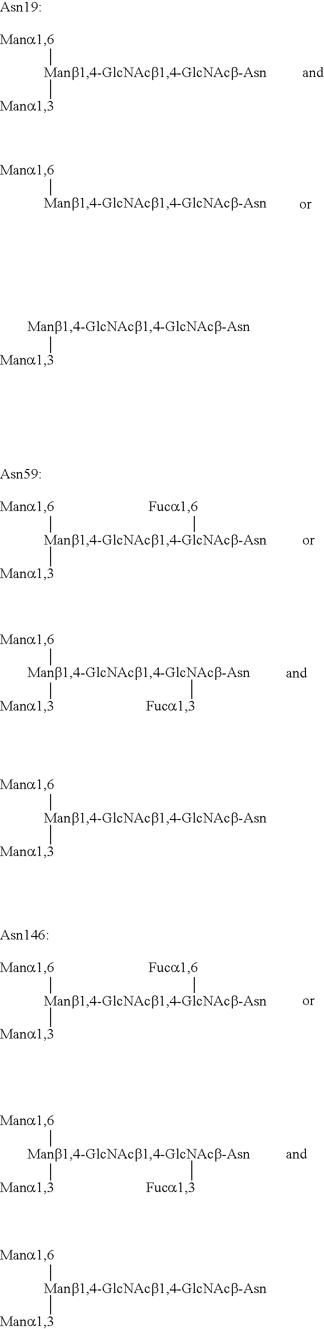

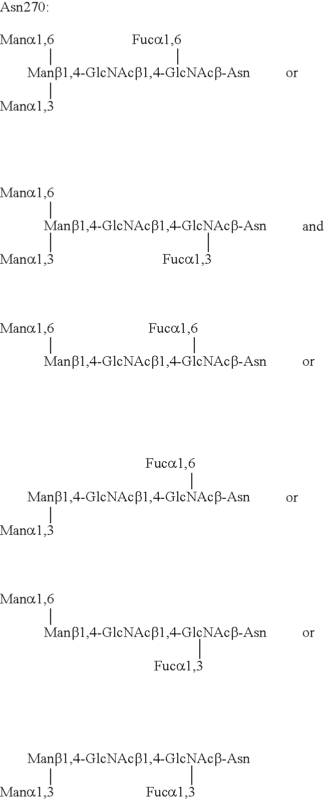

TABLE 7
Summary of the data obtained from exoglycosidase treatments of GCB glycopeptides.

| | | | | | Theoretical (T) |
|---|---|---|---|---|---|
| | | Suggested carbohydrate | Suggested carbohydrate | Suggested carbohydrate | and |
| | Suggested | structures, theoretical (T) | structures, theoretical (T) | structures, theoretical (T) | experimental |
| | carbohydrate | and | and | and | (E) |
| | structures and | experimental (E) | experimental (E) | experimental (E) | peptide mass |
| | theoretical | glycopeptide | glycopeptide | glycopeptide | after treatment |
| | glycopeptide | masses after treatment with | masses after treatment with | masses after treatment with | with |
| Position | masses | α(1-2,3,4) mannosidase | β(1-4) mannosidase | α(1-6) fucosidase | N-glycosidase A |
| Asn19 | Man₃GlcNAc₂ | ManGlcNAc₂ | GlcNAc₂ | GlcNAc₂ | T: 3608.57 Da |
| | 4500.89 Da | T: 4176.79 Da; E: 4176.05 Da | T: 4014.74 Da; E 4019.27 Da | T: 4014.74 Da; E: N.D. | E: N.D. |
| | Man₂GlcNAc₂ | | | | |
| | 4338.84 Da | | | | |
| Asn59 | Man₃GlcNAc₂Fuc | ManGlcNAc₂Fuc | GlcNAc₂Fuc | GlcNAc₂ | T: 2962.54 Da |
| | 4000.93 Da | T: 3676.83 Da; E: 3674.87 Da | T: 3514.78 Da; E: 3511.36 Da | T: 3368.72 Da; E: N.D. | E: N.D. |
| | Man₃GlcNAc₂ | ManGlcNAc₂ | GlcNAc₂ | | |
| | 3854.87 Da | T: 3530.77 Da; E: N.D. | T: 3368.72 Da; E: N.D. | | |
| Asn146 | Man₃GlcNAc₂Fuc | ManGlcNAc₂Fuc | GlcNAc₂Fuc | GlcNAc₂ | T: 2846.26 Da |
| | 3884.64 Da | T: 3560.54 Da; E: 3557.30 Da | T: 3398.49 Da; E: 3396.21 Da | T: 3252.43 Da; E: N.D. | E: N.D. |
| | Man₃GlcNAc₂ | ManGlcNAc₂ | GlcNAc₂ | | |
| | 3738.57 Da | T: 3414.48 Da; E: 3413.26 Da | T: 3252.43 Da; E: 3252.42 Da | | |
| Asn270 | Man₃GlcNAc₂Fuc | ManGlcNAc₂Fuc | GlcNAc₂Fuc | GlcNAc₂ | T: 1630.82 Da |
| | 2669.20 Da | T: 2345.1 Da; E: 2345.80 Da | T: 2183.05 Da, E: 2183.10 Da | T: 2036.99 Da | E: 1631.44 Da |
| | | | | E: 2036.59 Da/2183.64 Da | |
| | Man₂GlcNAc₂Fuc | | | | |
| | 2507.10 Da | | | | |

N.D., not determined.

Glycosylation of GCB Polypeptides of the Invention Expressed in Insect Cells

MALDI-TOF mass spectrometry was used to investigate the amount of carbohydrate attached to GCB polypeptides expressed in Sf9 cells.

The 7 GCB polypeptide variants investigated all contained additional potential N-glycosylation sites compared to wtGCB.

WtGCB contains 5 potential N-glycosylation sites of which only 4 are used.

The 7 GCB polypeptide variants were:

GC-36: ASPINATSPINAT (SEQ ID NO:145)-GCB,
GC-38: ASPINAT(SEQ ID NO:70)-GCB(K194N,K321N),
GC-60: ANNTNYTNWT(SEQ ID NO:140)-GCB,
GC-61: ATNITLNYTANTT(SEQ ID NO:142)-GCB,
GC-62: AANSTGNITINGT(SEQ ID NO:143)-GCB,
GC-63: AVNWTSNDTSNST(SEQ ID NO:145)-GCB,
and
GC-54: GCB-GGGG(SEQ ID NO:146)-Saposin C.

WtGCB:

The theoretical peptide mass of wtGCB is 55 591 Da. WtGCB has 5 potential N-glycosylation sites of which only 4 are used. As the two most common N-glycan structures on recombinant proteins expressed in Sf9 cells are Man₃GlcNAc₂Fuc and Man₃GlcNAc₂ having masses of 1038.38 Da and 892.31 Da, respectively, the expected mass of wtGCB carrying 4 N-glycans is between 59 159 Da and 59 743 Da.

MALDI-TOF mass spectrometry of wtGCB shows the broad peak typical of glycoproteins with a peak mass of 59.3 kDa in accordance with the expected mass of wtGCB carrying 4 N-glycans.

GC-36 (ASPINATSPINAT(SEQ ID NO:145)-GCB):

The theoretical peptide mass of GC-36 is 56 829 Da. The N-terminal extension contains two additional potential glycosylation sites at N5 and N11 compared to wtGCB. Assuming that the wtGCB part of the variant is glycosylated like wtGCB, the variant has 6 potential N-glycosylation sites.

As the two most common N-glycan structures on recombinant proteins expressed in Sf9 cells are Man₃GlcNAc₂Fuc and Man₃GlcNAc₂ having masses of 1038.38 Da and 892.31 Da, respectively, the expected mass of GC-36 carrying 4 N-glycans is between 60 397 Da and 60 981 Da, the expected mass of GC-36 carrying 5 N-glycans is between 61 289 Da and 62 019 Da, and the expected mass of GC-36 carrying 6 N-glycans is between 62 181 Da and 63 057 Da.

MALDI-TOF mass spectrometry of GC-36 shows a rather broad peak with a peak mass between 61.5 kDa and 62.9 kDa in accordance with the expected mass of GC-36 carrying either 5 or 6 N-glycans.

N-terminal amino acid sequence analysis of GC-36 showed that N5 is completely glycosylated while N11 is partially glycosylated in complete agreement with the result obtained using mass spectrometry.

GC-38 (ASPINAT(SEQ ID NO:70)-GCB(K194N,K321N)):

The theoretical peptide mass of GC-38 is 56 217 Da. The N-terminal extension contains one additional potential glycosylation sites at N5 compared to wtGCB. In addition, the substitutions of Lys194 and Lys321 with Asn-residues introduce two additional potential. N-glycosylation sites. Assuming that the wtGCB part of the variant is glycosylated like wtGCB, the variant has 7 potential N-glycosylation sites.

Based on the same considerations as those used for GC-36, the expected mass of GC-38 carrying 4 N-glycans is between 59 785 Da and 60 369 Da, the expected mass of GC-38 carrying 5 N-glycans is between 60 677 Da and 61 407 Da, the expected mass of GC-38 carrying 6 N-glycans is between 61 569 Da and 62 445 Da, and the expected mass of GC-38 carrying 7 N-glycans is between 62 461 Da and 63 483 Da.

MALDI-TOF mass spectrometry of GC-38 shows a major peak with a peak mass of 63.1 kDa in accordance with the expected mass of GC-38 carrying 7 N-glycans. In addition, a minor peak with a peak mass of 62.3 kDa is seen which corresponds to GC-38 carrying 6 N-glycans.

N-terminal amino acid sequence analysis of GC-38 showed that N5 is completely glycosylated.

GC-60 (ANNTNYTNWT(SEQ ID NO:140)-GCB):

The theoretical peptide mass of GC-60 is 56 770 Da. The N-terminal extension contains three additional potential glycosylation sites at N2, N5 and N8 compared to wtGCB. Assuming that the wtGCB part of the variant is glycosylated like wtGCB, the variant has 7 potential N-glycosylation sites.

Based on the same considerations as those used for GC-36 the expected mass of GC-60 carrying 4 N-glycans is between 60 338 Da and 60 922 Da, the expected mass of GC-60 carrying 5 N-glycans is between 61 230 Da and 61 960 Da, the expected mass of GC-60 carrying 6 N-glycans is between 62 122 Da and 62 998 Da, and the expected mass of GC-60 carrying 7 N-glycans is between 63 014 Da and 64 036 Da.

MALDI-TOF mass spectrometry of GC-60 shows two broad peaks with peak masses of 61.9 kDa and 62.8 kDa in accordance with the expected mass of GC-60 carrying either 5 or 6 N-glycans.

N-terminal amino acid sequence analysis of GC-60 showed that N2 is mainly glycosylated, N5 is completely glycosylated while N8 is only seldom glycosylated in acceptable agreement with the result obtained using mass spectrometry.

GC-61 (ATNITLNYTANTT(SEQ ID NO: 142)-GCB):

The theoretical peptide mass of GC-61 is 56 970 Da. The N-terminal extension contains three additional potential glycosylation sites at N3, N7 and N11 compared to wtGCB. Assuming that the wtGCB part of the variant is glycosylated like wtGCB, the variant has 7 potential N-glycosylation sites.

Based on the same considerations as used for GC-36, the expected mass of GC-61 carrying 4 N-glycans is between 60 538 Da and 61 122 Da, the expected mass of GC-61 carrying 5 N-glycans is between 61 430 Da and 62 160 Da, the expected mass of GC-61 carrying 6 N-glycans is between 62 322 Da and 63 198 Da, and the expected mass of GC-61 carrying 7 N-glycans is between 63 214 Da and 64 236 Da.

MALDI-TOF mass spectrometry of GC-61 shows a very broad peak with peak mass between 61.5 kDa and 63.0 kDa in accordance with the expected mass of GC-61 carrying either 5 or 6 N-glycans.

N-terminal amino acid sequence analysis of GC-61 showed that N3 is completely glycosylated while N7 and N11 are partially glycosylated in acceptable agreement with the result obtained using mass spectrometry.

GC-62 (AANSTGNITINGT(SEQ ID NO:143)-GCB):

The theoretical peptide mass of GC-62 is 56 806 Da. The N-terminal extension contains three additional potential glycosylation sites at N3, N7 and N11 compared to wtGCB. Assuming that the wtGCB part of the variant is glycosylated like wtGCB, the variant has 7 potential N-glycosylation sites.

Based on the same considerations as those used for GC-36, the expected mass of GC-62 carrying 4 N-glycans is between 60 374 Da and 60 958 Da, the expected mass of GC-62 carrying 5 N-glycans is between 61 266 Da and 61 996 Da, the expected mass of GC-62 carrying 6 N-glycans is between 62 158 Da and 63 034 Da, and the expected mass of GC-62 carrying 7 N-glycans is between 63 050 Da and 64 072 Da.

MALDI-TOF mass spectrometry of GC-62 shows two broad peaks with peak masses of 61.6 kDa and 62.7 kDa in accordance with the expected mass of GC-62 carrying either 5 or 6 N-glycans.

N-terminal amino acid sequence analysis of GC-62 showed that N3 is completely glycosylated while N7 and N11 are partially glycosylated in acceptable agreement with the result obtained using mass spectrometry.

GC-63 (AVNWTSNDTSNST(SEQ ID NO:145)-GCB):

The theoretical peptide mass of GC-63 is 56 969 Da. The N-terminal extension contains three additional potential glycosylation sites at N3, N7 and N11 compared to wtGCB. Assuming that the wtGCB part of the variant is glycosylated like wtGCB, the variant has 7 potential N-glycosylation sites.

Based on the same considerations as those used for GC-36, the expected mass of GC-63 carrying 4 N-glycans is between 60 537 Da and 61 121 Da, the expected mass of GC-63 carrying 5 N-glycans is between 61 429 Da and 62 159 Da, the expected mass of GC-63 carrying 6 N-glycans is between 62 321 Da and 63 197 Da, and the expected mass of GC-63 carrying 7 N-glycans is between 63 213 Da and 64 235 Da.

MALDI-TOF mass spectrometry of GC-63 shows a major peak with a peak mass of 61.9 kDa in accordance with the expected mass of GC-63 carrying 5 N-glycans. In addition, aminor peak with a peak mass of 62.9 kDa is seen which corresponds to GC-63 carrying 6 N-glycans.

N-terminal amino acid sequence analysis of GC-63 showed that N3 ans N7 are partially glycosylated. It was not possible to evaluate the glycosylation status of N11.

GC-54 (GCB-GGGG(SEQ ID NO:146)-Saposin C):

The theoretical peptide mass of GC-54 is 64 711 Da. The C-terminal saposin C extension contains one additional potential glycosylation sites compared to wtGCB. Assuming that the wtGCB part of the variant is glycosylated like wtGCB, the variant has 5 potential N-glycosylation sites.

Based on the same considerations as those used for GC-36, the expected mass of GC-54 carrying 4 N-glycans is between 68 279 Da and 68 863 Da while the expected mass of GC-54 carrying 5 N-glycans is between 69 171 Da and 69 901 Da.

MALDI-TOF mass spectrometry of GC-54 shows a rather broad peak with a peak mass of 68.4 kDa in accordance with the expected mass of GC-54 carrying 4 N-glycans. Thus, the N-glycosylation site in the saposin C extension is probably not used.

Furthermore, insect cell expressed N-terminally extended glycosylated polypeptide (GC-6 and GC-13) was subjected to N-terminal amino acid sequence analysis (using Procize from PE Biosystems, Foster City, Calif.). The sequencing cycle was blank for the Asn residue in both ANIT and ASPINAT N-terminal peptide additions, demonstrating that the introduced glycosylation site is glycosylated.

When subjecting GC-13 to mass spectrophometry using the MALDI-TOF techniques on the Voyager DERP instrument (from PE-Biosystems, Foster City, Calif.) the following results were obtained:

The wildtype and ASPINAT-extended wildtype expressed in insect cells gave average masses very close to the calculated mass of 59,727 Da and 61,421 Da, respectively, assuming that four glycosylation sites were occupied by the carbohydrates FucGlcNAc₂Man₃.

### Example 9

Expression of GCB in CHO lec1

The wtGCB-cDNA was isolated from pGC12 by digestion with NheI and XbaI, and cloned into pcDNA3.1/Hygro+(Invitrogen, Carlsbad, Calif., USA) digested with NheI and XbaI. The resulting plasmid was then transfected into CHO lec1 cells (Mutant clonal derivative of Chinese hamster ovary CHO clone pro-5) (available from the American Type Culture Collection 10801 University Boulevard, Manassas, Va. 20110-2209, USA Item number CRL-1735) using Lipofectamin 2000 (Cat no. 11668-019 Gibco BRL, Life Technologies). The day after transfection GCB activity in the transfecting medium and the cells were measured, using the PNP GCB Activity Assay, with the following result: Medium: 0.03 U/L; Cells: 2.99 U/L.

The medium was then replaced with a selective medium DMEM/F12 (Cat no. 21041-025 Gibco BRL, Life Technologies)+10%FBS (Fetal Bovine Serum Cat no. 02-701 F Bio-whittaker Europe B-4800 verviers Belgium)+100 U/ml Penicillin/100 μg/ml Streptomycin (Cat no. DE17-602E Bio-whittaker Europe B-4800 verviers Belgium)+400 μg/ml Hygromycin (Hygromycin B in PBS 50 mg/ml Cat no. 10687-010 Gibco BRL, Life Technologies). When cells were 100% confluent in the selective medium, the GCB activity in the medium and the cells were measured as above resulting in the following activities: Medium: 0.05 U/L; Cells: 1.49 U/L.

Independent clones were selected in microtiter plates and 30 clones which grew in the selective medium were measured in the GCB Activity Assay, Three high-producing clones were selected for growth in T flasks. By lowering the pH of the medium to 6.5 and adding DTT to a molar concentration of 0.2 to 1.0 mM a relative high amount of GCB is secreted with an N-glycosylation structure believed to comprise 5 exposed mannose residues (a similar glycosylation structure was described for the G glycoprotein of vesicular stomatitis virus expressed in the same cell line as described in Robertson et al., Cell 13, pp. 515-526, 1978).

While the foregoing invention has been described in some detail for purposes of clarity and understanding, it will be clear to one skilled in the art from a reading of this disclosure that various changes in form and detail can be made without departing from the true scope of the invention. For example, all the techniques, methods, compositions, apparatus and systems described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A polypeptide selected from the group consisting of lysosomal enzymes and lysosomal enzyme activators, comprising at least one introduced glycosylation site as compared to a corresponding parent enzyme or activator.

2. The polypeptide according to claim 1, wherein the glycosylation site is introduced into the amino acid sequence of the mature form of the parent lysosomal enzyme or activator.

3. The polypeptide according to claim 2, wherein the glycosylation site is introduced into a surface exposed position of the parent enzyme or activator.

4. The polypeptide according to claim 1, wherein the glycosylation site is introduced into a position of the parent enzyme or activator that is occupied by a charged amino acid residue selected from the group consisting of E, D, R, K and H, or a position that is located between position −4 and +4 relative to a lysine residue.

5. The polypeptide according to claim 2, comprising at least 2-10 introduced glycosylation sites.

6. The polypeptide according to claim 1, lacking at least one glycosylation site present in the parent enzyme or activator.

7. The polypeptide according to claim 1, wherein the lysosomal enzyme or activator comprises an N-terminal or C-terminal peptide addition as compared to the corresponding parent enzyme or activator, the peptide addition comprising or contributing to at least one glycosylation site.

8. The polypeptide according to claim 7, wherein the peptide addition comprises 1-500 amino acid residues.

9. The polypeptide according to claim 7, wherein the peptide addition comprises 1-20 or 1-10 glycosylation sites.

10. The polypeptide according to claim 1, wherein the glycosylation site is an in vivo glycosylation site or an N-glycosylation site.

11. The polypeptide according to claim 7, wherein the peptide addition comprises a peptide sequence selected from the group consisting of INAT/S, GNIT/S, VNIT/S, SNIT/S, ASNIT/S (SEQ ID NO:7), NIT/S, SPINAT/S (SEQ ID NO:8), ASPINAT/S (SEQ ID NO:9), ANIT/SANIT/SANI (SEQ ID NO:10), ANIT/SGSNIT/SGSNIT/S (SEQ ID NO: 11), ASNST/SNNGT/SLNAT/S (SEQ ID NO: 12), ANHT/SNET/SNAT/S (SEQ ID NO: 13), GSPINAT/S (SEQ ID NO: 14), ASPINAT/SSPINAT/S (SEQ ID NO: 15), ANNT/SNYT/SNWT/S (SEQ ID NO:16), ATNIT/SLNYT/SANT/ST (SEQ ID NO:17), AANST/SGNIT/SINGT/S (SEQ ID NO:18), AVNWT/SSNDT/SSNST/S (SEQ ID NO:19), GNAT/S, AVNWT/SSNDT/SSNST/S (SEQ ID NO:20), ANNT/SNYT/SNST/S (SEQ ID NO:21), and ANNTNYTNWT (SEQ ID NO:22), wherein T/S is either a T or an S residue, preferably a T residue.

12. The polypeptide according to claim 10, wherein the peptide addition has an N residue in position −2 or −1, and the lysosomal enzyme or activator has a T or an S residue in position +1 or +2, respectively, the residue numbering being made relative to the N-terminal amino acid residue of the lysosomal enzyme or activator.

13. A chimeric polypeptide comprising a lysosomal enzyme unit linked to at least one unit of an activator for said enzyme.

14. The polypeptide according to claim 13, wherein the enzyme unit and the activator unit(s) are linked by a peptide bond or peptide linker.

15. A chimeric polypeptide comprising a lysosomal enzyme unit linked to at least one targeting polypeptide unit, the targeting polypeptide being capable of targeting phagocytic cells.

16. The polypeptide according to claim 1, wherein the lysosomal enzyme or activator is one that binds to a mannose receptor.

17. The polypeptide according to claim 1, wherein the lysosomal enzyme is selected from the group consisting of glucocerebrosidase (GCB), α-L-iduronidase, acid α-glucosidase, α-galactosidase, acid sphingomyelinase, galactocerebrosidase, arylsulphatase A, sialidase, and hexosaminidase.

18. The polypeptide according to claim 1, wherein the activator is Saposin A, Sapocin B, Sapocin C, Sapocin D, or GM-2 activator.

19. The polypeptide according to any of claim 1, wherein the lysosomal enzyme is a glucocerebrosidase (GCB) polypeptide.

20. The polypeptide according to claim 19, wherein the glycosylation site is an N-glycosylation site and the polypeptide comprises one or more substitutions, relative to the amino acid sequence shown in SEQ ID NO: 1, selected from the group consisting of K7N+F9T, K7N+*9T, K7N+*9S, K7N+F9S, K74N+Q76T, K74N+Q76S, K77N+K79T, K77N+K79S, K79N+F81T, K79N+F81S, K106N+Y108T, K106N+Y108S, K155N+K157T, K155N+K157S, K157N+P159T, K157N+P159S, K186N+N188T, K186N+N188S, K193N+S195T, K194N, K194T, K198N+Q200T, K198N+Q200S, K215N+L217T, K215N+L217S, E222N+K224T, K224N+Q226T, K224N+Q226S, K293N+L295T, K293N+L295S, K303N+V305T, K303N+V305S, K321N, K321N+T323S, K346N+W348T, K346N+W348S, K408N, K408N+T410S, K413N+P415T, K413N+P415S, K425N+1427T, K425N+1427S, K441N+D443T, K441N+D443S, K466N+V468T, K466N+V468S, K473N+P475T and K473N+P475S.

21. A polypeptide according to claim 19, wherein the glycosylation site is an N-glycosylation site and one or more amino acid residue of the parent GCB polypeptide is selected from the group consisting of P6, G10, Y11, C23, T36, Y40, T43, E50, A95, L105, Y108, M133, D137, P171, L175, W179, K194, L240, A269, E235, F337, V343, E349, L354, Q362, S364, V398, H422, E429, V437, D453, R463, T482, G486, P28, L34, E41, T61, L66, A84, I130, T132, A136, S181, E152, P178, L185, H206, G255, A291, G250, V295, K321, G325, P332, I367, G377, D405, K408, P465, L480 and I489 of the amino acid sequence shown in SEQ ID NO: 1 substituted with an asparagine residue.

22. The polypeptide according to claim 19, wherein the glycosylation site is an in vitro glycosylation site selected from the group consisting of the N-terminal amino acid residue of the polypeptide, the C-terminal residue of the polypeptide, lysine, cysteine, arginine, glutamine, aspartic acid, glutamic acid, serine, tyrosine, histidine, phenylalanine and tryptophan.

23. The polypeptide according to claim 22, wherein the in vitro glycosylation site is a lysine residue.

24. The polypeptide according to claim 23, wherein one or more of the amino acid residues of wtGCB (SEQ ID NO 1) selected from the group consisting of R2, R39, R44, R47, R48, R120, R131, R163, R170, R211, R257, R262, R277, R285, R339, R353, R359, R395, R433, R463, R495, R496, H60, H145, H162, H206, H223, H255, H273, H274, H290, H306, H311, H328, H365, H374, H419, H422, H451, H490, D24, D27, D87, D127, D137, D140, D141, D153, D203, D218, D258, D263, D282, D283, D298, D358, D380, D399, D405, D409, D443, D445, D453, D467, D474, E41, E50, E72, E111, E112, E151, E152, E222, E233, E235, E254, E300, E326, E340, E349, E388, E429, and E481 have been replaced with a lysine residue.

25. The polypeptide according to claim 22, further lacking an in vitro glycosylation site present in wtGCB.

26. The polypeptide according to claim 25, wherein a lysine residue present in wtGCB is substituted with arginine or is deleted from one or more positions selected from the group consisting of K7, K74, K77, K79, K106, K155, K157, K186, K193, K197, K215, K224, K293, K303, K321, K346, K408, K413, K425, K441, K466 and K473 of the amino acid sequence shown in SEQ ID NO: 1.

27. A GCB polypeptide comprising a modification at any of amino acid residues 132-139 relative to SEQ ID NO 1, resulting in reduced susceptibility to proteolytic degradation.

28. The GCB polypeptide according to claim 27, wherein a glycosylation site is introduced into any of positions 132-139.

29. The GCB polypeptide according to claim 27, comprising the mutation A136N, A135P or A136P.

30. A chimeric polypeptide comprising at least one unit of a polypeptide targeting phagocytic cells, macrophages, or macrophage like cells, and a GCB polypeptide unit.

31. A chimeric polypeptide comprising a GCB polypeptide unit and at least one Saposin C polypeptide and/or a Saposin A polypeptide unit.

32. The chimeric polypeptide according to claim 30, wherein the different polypeptide constituents are linked with a peptide bond or a peptide linker.

33. The chimeric polypeptide according to claims 30, wherein the GCB polypeptide is a polypeptide according to claim 19 or a wtGCB with an amino acid sequence included in SEQ ID NO: 1.

34. The polypeptide according to claim 1, which polypeptide is glycosylated.

35. The glycosylated polypeptide according to claim 34, comprising at least one oligosaccharide chain comprising an exposed mannose residue.

36. The polypeptide according to claim 34, which polypeptide has a glycosylation profile characteristic of that provided by expression in an invertebrate cell.

37. The polypeptide according to claim 34, which polypeptided has the glycosylation profiled characteristic of that provided by expression in a yeast, insect, or plant cell.

38. The polypeptide according to claim 37, wherein the insect cell is a Lepidoptora cell line.

39. The polypeptide according to claim 34, wherein at least one oligosaccharide chain has the structure
Asn-N-N-M-M₂
wherein Asn indicates the Asn residue of the polypeptide to which the oligosaccharide chain is attached, N an N-acetylglucosamine residue, and M-M₂ three mannose residues two of which are linked to the same mannose.

40. The polypeptide according to claim 34, which polypeptide is expressed from a mammalian cell line and subsequently modified by sequential treatment with neuramidase, galactosidase and β-N acetylglucosaminidase, thereby providing at least one exposed mannose residue.

41. The polypeptide according to claim 34, comprising comprising 1-10 oligosaccharide moieties.

42. The polypeptide according to claim 36, which polypeptide is expressed from a cell producing a fucose-containing oligosaccharide structure, wherein said polypeptide, subsequent to expression, is treated with a fucosidase.

43. The polypeptide according to claim 1, which has at least one of the following properties:
increased affinity for a mannose receptor or other carbohydrate receptor,
increased serum half-life,
increased functional in vivo half-life,
increased in vivo bioactivity,
reduced immunogenicity,
increased resistance to proteolytic cleavage, or
increased targeting to or uptake in phagocytic cells or a suborganel compartment thereof.

44. The polypeptide according to claim 19, which exhibits increased in vivo activity relative to a wildtype GCB (wtGCB).

45. A nucleotide sequence encoding a polypeptide according to claim 1.

46. An expression vector comprising a nucleotide sequence according to claim 45.

47. A host cell transformed or transfected with a nucleotide sequence according to claim 45, or an expression vector according to claim 46.

48. The host cell according to claim 47, which is an invertebrate cell such as an insect cell, a yeast cell or a plant cell, or a mammalian cell, in particular a glycosylation mutant thereof.

49. The cell line according to claim 48, wherein the GCB polypeptide is a wtGCB or a variant or truncated form thereof or a GCB polypeptide comprising at least one introduced glycosylation site as compound to a wild-type GCB.

50. A CHO lec1 cell line comprising a heterologous nucleotide sequence encoding a lysosomal enzyme or a lysosomal enzyme activator.

51. A method of producing a polypeptide according to claim 1, comprising culturing the host cell according to claim 47 under conditions permitting expression of the polypeptide and recovering the polypeptide from the culture.

52. The method according to claim 51, further comprising subjecting the optionally glycosylated polypeptide to in vitro glycosylation.

53. A method of improving at least one property of a lysosomal enzyme, which method comprises introducing an additional glycosylation site into the lysosomal enzyme to be improved, and producing the modified lysosomal enzyme under conditions ensuring that the enzyme is glycosylated.

54. The method according to claim 53, wherein the lysosomal enzyme is a GCB polypeptide.

55. The method according to claim 53, wherein the improved property is any of those mentioned in claim 43.

56. A pharmaceutical composition comprising a polypeptide according to claim 1 and a pharmaceutically acceptable diluent, carrier or excipient.

57. A method of treating Gaucher's disease, in which an effective amount of a GCB polypeptide according to claim 19, a Saposin C polypeptide or a chimeric polypeptide thereof is administered to a patient in need thereof.

58. The use of a nucleotide sequence according to claim 45 in gene therapy, the nucleotide sequence encoding a lysosomal enzyme or activator thereof with at least one introduced in vivo glycosylation site as compared to a parent, naturally-occurring enzyme or activator.


SEQUENCE LISTING (147 sequences)

SEQ ID NO: 1 - protein, 497 aa, Homo sapiens
  MOD_RES (495): R or H
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWXRQ

SEQ ID NO: 2 - DNA, 1551 bp, Homo sapiens
atggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagcactgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttaattgcccgtgacctaggtcctaccctcgccaacagtactcaccacaatgtccgcctactcatgctggatgaccaacgcttgctgctgccccactgggcaaaggtggtgctgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccatttcagcaagttcattcctgagggctcccagagagtggggctggttgccagtcagaagaacgacctggacgcagtggcattgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 3 - protein, 80 aa, Homo sapiens
SDVYCEVCEFLVKEVTKLIDNNKTEKEILDAFDKMCSKLPKSLSEECQEVVDTYGSSILSILLEEVSPELVCSMLHLCSG

SEQ ID NO: 4 - protein, 592 aa, Artificial Sequence [sequence omitted]

SEQ ID NO: 5 - protein, 6 aa, Artificial Sequence
  Description of Artificial Sequence Synthetic peptide
AATPAP

SEQ ID NO: 6 - protein, 8 aa, Artificial Sequence
  Description of Artificial Sequence Synthetic peptide
TGRGDSPA

SEQ ID NO: 7 - protein, 5 aa, Artificial Sequence
  Description of Artificial Sequence Synthetic peptide
ASNIX

SEQ ID NO: 8 - protein, 6 aa, Artificial Sequence
  Description of Artificial Sequence Synthetic peptide
SPINAX

SEQ ID NO: 9 - protein, 7 aa, Artificial Sequence
  Description of Artificial Sequence Synthetic peptide
ASPINAX

SEQ ID NO: 10 - protein, 11 aa, Artificial Sequence
  Description of Artificial Sequence Synthetic peptide
ANIXANIXANI

SEQ ID NO: 11 - protein, 14 aa, Artificial Sequence
  Description of Artificial Sequence Synthetic peptide
ANIXGSNIXGSNIX

SEQ ID NO: 12 - protein, 13 aa, Artificial Sequence
  Description of Artificial Sequence Synthetic peptide
ASNSXNNGXLNAX

SEQ ID NO: 13 - protein, 10 aa, Artificial Sequence
  Description of Artificial Sequence Synthetic peptide
ANHXNEXNAX

SEQ ID NO: 14 - protein, 7 aa, Artificial Sequence
  Description of Artificial Sequence Synthetic peptide
GSPINAX

SEQ ID NO: 15 - protein, 13 aa, Artificial Sequence
  Description of Artificial Sequence Synthetic peptide
ASPINAXSPINAX

SEQ ID NO: 16 - protein, 10 aa, Artificial Sequence
  Description of Artificial Sequence Synthetic peptide
ANNXNYXNWX

SEQ ID NO: 17 - protein, 13 aa, Artificial Sequence
  Description of Artificial Sequence Synthetic peptide
ATNIXLNYXANXT

SEQ ID NO: 18 - protein, 13 aa, Artificial Sequence
  Description of Artificial Sequence Synthetic peptide
AANSXGNIXINGX

SEQ ID NO: 19 - protein, 13 aa, Artificial Sequence
  Description of Artificial Sequence Synthetic peptide
AVNWXSNDXSNSX

SEQ ID NO: 20 - protein, 13 aa, Artificial Sequence
  Description of Artificial Sequence Synthetic peptide
AVNWXSNDXSNSX

SEQ ID NO: 21 - protein, 10 aa, Artificial Sequence
  Description of Artificial Sequence Synthetic peptide
ANNXNYXNSX

SEQ ID NO: 22 - protein, 10 aa, Artificial Sequence
  Description of Artificial Sequence Synthetic peptide
ANNTNYTNWT

SEQ ID NO: 23 - protein, 15 aa, Artificial Sequence
  Description of Artificial Sequence Linker
GGGGSGGGGSGGGGS

SEQ ID NO: 24 - DNA, 35 bp, Artificial Sequence
  Description of Artificial Sequence Primer
cgcagatctgatggctggcagcctcacaggattgc

SEQ ID NO: 25 - DNA, 37 bp, Artificial Sequence
  Description of Artificial Sequence Primer
ccggaattcccatcactggcgacgccacaggtaggtg

SEQ ID NO: 26 - DNA, 35 bp, Artificial Sequence
  Description of Artificial Sequence Primer
acgcgagctcgcccctgcatccctaaaagcttcgg

SEQ ID NO: 27 - DNA, 54 bp, Artificial Sequence
  Description of Artificial Sequence Primer
gcgttgacggcagtcagagttgacagaagggccagccagcaaaggatagtcatg

SEQ ID NO: 28 - DNA, 62 bp, Artificial Sequence
  Description of Artificial Sequence Primer
ctagcatgactatcctttgctggctggcccttctgtcaactctgactgccgtcaacgcagct

SEQ ID NO: 29 - DNA, 48 bp, Artificial Sequence
  Description of Artificial Sequence Primer
cctgctactgctcccagcagcagtgaaagagtccaaagtggcagcatg

SEQ ID NO: 30 - DNA, 56 bp, Artificial Sequence
  Description of Artificial Sequence Primer
ctagcatgctgccactttggactctttcactgctgctgggagcagtagcaggagct

SEQ ID NO: 31 - DNA, 21 bp, Artificial Sequence
  Description of Artificial Sequence Primer
cagctggccatgggtacccgg

SEQ ID NO: 32 - DNA, 25 bp, Artificial Sequence
  Description of Artificial Sequence Primer
ccctccaaatcccttcactttctgg

SEQ ID NO: 33 - DNA, 24 bp, Artificial Sequence
  Description of Artificial Sequence Primer
gagtttttggttcttgccgggtcc

SEQ ID NO: 34 - DNA, 29 bp, Artificial Sequence
  Description of Artificial Sequence Primer
ccttcactgtctggttcttctgttctggc

SEQ ID NO: 35 - DNA, 29 bp, Artificial Sequence
  Description of Artificial Sequence Primer
ccgtcacgttctggaacttctgttctggc

SEQ ID NO: 36 - DNA, 29 bp, Artificial Sequence
  Description of Artificial Sequence Primer
ccaaaccagaccttccagaaagtgaaggg

SEQ ID NO: 37 - DNA, 29 bp, Artificial Sequence
  Description of Artificial Sequence Primer
ccttcgttttgttgaacttctgttctggc

SEQ ID NO: 38 - DNA, 29 bp, Artificial Sequence
  Description of Artificial Sequence Primer
ccagaaaacaagacccagaaagtgaaggg

SEQ ID NO: 39 - DNA, 32 bp, Artificial Sequence
  Description of Artificial Sequence Primer
ccggttccgttttcagagaagtacgatttaag

SEQ ID NO: 40 - DNA, 29 bp, Artificial Sequence
  Description of Artificial Sequence Primer
ccagaacagaagttccagaaagtgaaggg

SEQ ID NO: 41 - DNA, 29 bp, Artificial Sequence
  Description of Artificial Sequence Primer
attccagtttcattgaagtacgatttaag

SEQ ID NO: 42 - DNA, 29 bp, Artificial Sequence
  Description of Artificial Sequence Primer
ggtaccttcagccgctatgagagtacacg

SEQ ID NO: 43 - DNA, 29 bp, Artificial Sequence
  Description of Artificial Sequence Primer
attccttcggtagagttgtacgatttaag

SEQ ID NO: 44 - DNA, 29 bp, Artificial Sequence
  Description of Artificial Sequence Primer
ggtaacttcagccgctatgagagtacacg

SEQ ID NO: 45 - DNA, 29 bp, Artificial Sequence
  Description of Artificial Sequence Primer
attccttcttcagagaagttcgatttaag

SEQ ID NO: 46 - DNA, 29 bp, Artificial Sequence
  Description of Artificial Sequence Primer
ggtaccaacagcacctatgagagtacacg

SEQ ID NO: 47 - DNA, 29 bp, Artificial Sequence
  Description of Artificial Sequence Primer
ggtgtcttgttcttggtatcttcctctgg

SEQ ID NO: 48 - DNA, 29 bp, Artificial Sequence
  Description of Artificial Sequence Primer
ggtaccttcaaccgcaccgagagtacacg

SEQ ID NO: 49 - DNA, 29 bp, Artificial Sequence
  Description of Artificial Sequence Primer
ggtatcttggtcttgttatcttcctctgg

SEQ ID NO: 50 - DNA, 29 bp, Artificial Sequence
  Description of Artificial Sequence Primer
ggtaccttcagcaactatactagtacacg

SEQ ID NO: 51 - DNA, 29 bp, Artificial Sequence
  Description of Artificial Sequence Primer
ggtatcttgagcgtggtattttcctctgg

SEQ ID NO: 52 - DNA, 29 bp, Artificial Sequence
  Description of Artificial Sequence Primer
ggtaccttcagccgcaatgagagtacacg

SEQ ID NO: 53 - DNA, 29 bp, Artificial Sequence
  Description of Artificial Sequence Primer
ggtatcttgagcttggtatcttcctctgg

SEQ ID NO: 54 - DNA, 29 bp, Artificial Sequence
  Description of Artificial Sequence Primer
ccagagaacgataccaagctcaagatacc

SEQ ID NO: 55 - DNA, 38 bp, Artificial Sequence
  Description of Artificial Sequence Primer
ctgggtgtagttgtccccgggctgtcccttgagtgacc

SEQ ID NO: 56 - DNA, 29 bp, Artificial Sequence
  Description of Artificial Sequence Primer
ccaaacgaaactaccaagctcaagatacc

SEQ ID NO: 57 - DNA, 35 bp, Artificial Sequence
  Description of Artificial Sequence Primer
gtgggtgatgttcccgggctgtcccttgagtgacc

SEQ ID NO: 58 - DNA, 29 bp, Artificial Sequence
  Description of Artificial Sequence Primer
ccagaggaagataccaagctcaagatacc

SEQ ID NO: 59 - DNA, 35 bp, Artificial Sequence
  Description of Artificial Sequence Primer
gtggtagatgtccccgggctgtcccttgagtgacc

SEQ ID NO: 60 - DNA, 35 bp, Artificial Sequence
  Description of Artificial Sequence Primer
ggtcaaacaagacacagcccggggacatctaccac

SEQ ID NO: 61 - DNA, 29 bp, Artificial Sequence
  Description of Artificial Sequence Primer
ctgtcagcaccgtcttgttccagtggggc

SEQ ID NO: 62 - DNA, 35 bp, Artificial Sequence
  Description of Artificial Sequence Primer
ggtcactcaagggacagcccggggacatctaccac

SEQ ID NO: 63 - DNA, 29 bp, Artificial Sequence
  Description of Artificial Sequence Primer
ctgtggtcacgttctttgcccagtggggc

SEQ ID NO: 64 - DNA, 29 bp, Artificial Sequence
  Description of Artificial Sequence Primer
gcccaactggactaaggtggtgctgacag

SEQ ID NO: 65 - DNA, 29 bp, Artificial Sequence
  Description of Artificial Sequence Primer
ctgtcaggttcacctttgcccagtggggc

SEQ ID NO: 66 - DNA, 29 bp, Artificial Sequence
  Description of Artificial Sequence Primer
gccccacaccgcaaccgtggtgctgacag

SEQ ID NO: 67 - DNA, 29 bp, Artificial Sequence
  Description of Artificial Sequence Primer
ctgtcagcaccacctttgcccagtggggc

SEQ ID NO: 68 - DNA, 29 bp, Artificial Sequence
  Description of Artificial Sequence Primer
gccccactgggcaaaggtggtgctgacag

SEQ ID NO: 69 - protein, 4 aa, Artificial Sequence
  Description of Artificial Sequence N-terminal peptide addition
ANIT

SEQ ID NO: 70 - protein, 7 aa, Artificial Sequence
  Description of Artificial Sequence N-terminal peptide addition
ASPINAT

SEQ ID NO: 71 - DNA, 48 bp, Artificial Sequence
  Description of Artificial Sequence Primer
tgggcatcaggtgccaacattacagcccgcccctgcatccctaaaagc

SEQ ID NO: 72 - DNA, 24 bp, Artificial Sequence
  Description of Artificial Sequence Primer
tttactgttttcgtaacagttttg

SEQ ID NO: 73 - DNA, 48 bp, Artificial Sequence
  Description of Artificial Sequence Primer
gcaggggcgggctgtaatgttggcacctgatgcccacgacactgcctg

SEQ ID NO: 74 - protein, 13 aa, Artificial Sequence
  Description of Artificial Sequence Synthetic peptide
AXNXTXNXTXNXT

SEQ ID NO: 75 - protein, 10 aa, Artificial Sequence
  Description of Artificial Sequence Synthetic peptide
ANXTNXTNXT

SEQ ID NO: 76 - DNA, 81 bp, Artificial Sequence
  modified_base (1)..(81): “n” represents a, t, c, g, other or unknown
gtgtcgtgggcatcaggtgccnnsaaydnsachdnsaaydnsachdnsaaydnsachgcccgcccctgcatccctaaaagc

SEQ ID NO: 77 - DNA, 27 bp, Artificial Sequence
  Description of Artificial Sequence Primer
ggcacctgatgcccacgacactgcctg

SEQ ID NO: 78 - DNA, 68 bp, Artificial Sequence
  Description of Artificial Sequence Primer
cgtgggcatcaggtgccaacnnnachaaynnnachaaynnnachgcccgcccctgcatccctaaaagc

SEQ ID NO: 79 - DNA, 30 bp, Artificial Sequence
  Description of Artificial Sequence Primer
gttggcacctgatgcccacgacactgcctg

SEQ ID NO: 80 - protein, 13 aa, Artificial Sequence
  Description of Artificial Sequence Synthetic peptide
AFNXTLNKTWNXT

SEQ ID NO: 81 - protein, 13 aa, Artificial Sequence
  Description of Artificial Sequence Synthetic peptide
TMNNTWNWTWNWT

SEQ ID NO: 82 - protein, 13 aa, Artificial Sequence
  Description of Artificial Sequence Synthetic peptide
ALNSTGNLTVDGT

SEQ ID NO: 83 - protein, 13 aa, Artificial Sequence
  Description of Artificial Sequence Synthetic peptide
ASNSTFNLTENLT

SEQ ID NO: 84 - protein, 12 aa, Artificial Sequence
  Description of Artificial Sequence Synthetic peptide
TRNVTINCTNST

SEQ ID NO: 85 - protein, 13 aa, Artificial Sequence
  Description of Artificial Sequence Synthetic peptide
ALNWTYNGTKNVT

SEQ ID NO: 86 - protein, 13 aa, Artificial Sequence
  Description of Artificial Sequence Synthetic peptide
AANWTVNFTGNFT

SEQ ID NO: 87 - protein, 12 aa, Artificial Sequence
  Description of Artificial Sequence Synthetic peptide
AXNXTVNSTNVT

SEQ ID NO: 88 - protein, 13 aa, Artificial Sequence
  Description of Artificial Sequence Synthetic peptide
ANNFTFNGTLNLT

SEQ ID NO: 89 - protein, 13 aa, Artificial Sequence
  Description of Artificial Sequence Synthetic peptide
AGNWTANVTVNVT

SEQ ID NO: 90 - protein, 13 aa, Artificial Sequence
  Description of Artificial Sequence Synthetic peptide
AGNSTSNVTGNWT

SEQ ID NO: 91 - protein, 13 aa, Artificial Sequence
  Description of Artificial Sequence Synthetic peptide
AVNSTMNIHAIPP

SEQ ID NO: 92 - protein, 13 aa, Artificial Sequence
  Description of Artificial Sequence Synthetic peptide
AGNGTVNGTINGT

SEQ ID NO: 93 - protein, 13 aa, Artificial Sequence
  Description of Artificial Sequence Synthetic peptide
AVNSTGNXTGNWT

SEQ ID NO: 94 - protein, 12 aa, Artificial Sequence
  Description of Artificial Sequence Synthetic peptide
AGNGTNGTSNLT

SEQ ID NO: 95 - protein, 13 aa, Artificial Sequence
  Description of Artificial Sequence Synthetic peptide
AMNSTKNSTLNIT

SEQ ID NO: 96 - protein, 10 aa, Artificial Sequence
  Description of Artificial Sequence Synthetic peptide
AFNYTSKNST

SEQ ID NO: 97 - protein, 13 aa, Artificial Sequence
  Description of Artificial Sequence Synthetic peptide
AVNATMNWTANGT

SEQ ID NO: 98 - protein, 13 aa, Artificial Sequence
  Description of Artificial Sequence Synthetic peptide
ASNSTNNGTLNAT

SEQ ID NO: 99 - protein, 13 aa, Artificial Sequence
  Description of Artificial Sequence Synthetic peptide
ARNKTKNFTINLT

SEQ ID NO: 100 - protein, 12 aa, Artificial Sequence
  Description of Artificial Sequence Synthetic peptide
APNITNDTVNMT

SEQ ID NO: 101 - protein, 13 aa, Artificial Sequence
  Description of Artificial Sequence Synthetic peptide
AQNKTFNFTMNCT

SEQ ID NO: 102 - protein, 13 aa, Artificial Sequence
  Description of Artificial Sequence Synthetic peptide
ALNVTWNCTLNLT

SEQ ID NO: 103 - protein, 10 aa, Artificial Sequence
  Description of Artificial Sequence Synthetic peptide
ALNTTWTNLT

SEQ ID NO: 104 - protein, 10 aa, Artificial Sequence
  Description of Artificial Sequence Synthetic peptide
ANTTNFTNET

SEQ ID NO: 105 - protein, 10 aa, Artificial Sequence
  Description of Artificial Sequence Synthetic peptide
ANWTNRTNCT

SEQ ID NO: 106 - protein, 10 aa, Artificial Sequence
  Description of Artificial Sequence Synthetic peptide
ANWTNFTNWT

SEQ ID NO: 107 - protein, 10 aa, Artificial Sequence
  Description of Artificial Sequence Synthetic peptide
PTGLIGTNFT

SEQ ID NO: 108 - protein, 10 aa, Artificial Sequence
  Description of Artificial Sequence Synthetic peptide
ANWTNKTNFT

SEQ ID NO: 109 - protein, 10 aa, Artificial Sequence
  Description of Artificial Sequence Synthetic peptide
ANNTNLTNAT

SEQ ID NO: 110 - protein, 10 aa, Artificial Sequence
  Description of Artificial Sequence Synthetic peptide
ANYTNWTNFT

SEQ ID NO: 111 - protein, 10 aa, Artificial Sequence
  Description of Artificial Sequence Synthetic peptide
ANTTNQTNDT

SEQ ID NO: 112 - protein, 10 aa, Artificial Sequence
  Description of Artificial Sequence Synthetic peptide
ANRTNWTNTT

SEQ ID NO: 113 - protein, 10 aa, Artificial Sequence
  Description of Artificial Sequence Synthetic peptide
PTATNHTNST

SEQ ID NO: 114 - protein, 10 aa, Artificial Sequence
  Description of Artificial Sequence Synthetic peptide
ANWTNQTNQT

SEQ ID NO: 115 - protein, 10 aa, Artificial Sequence
  Description of Artificial Sequence Synthetic peptide
ANWTNWTNAT

SEQ ID NO: 116 - protein, 10 aa, Artificial Sequence
  Description of Artificial Sequence Synthetic peptide
ANFTNKTNMT

SEQ ID NO: 117 - protein, 10 aa, Artificial Sequence
  Description of Artificial Sequence Synthetic peptide
ANHTNETNAT

SEQ ID NO: 118 - protein, 10 aa, Artificial Sequence
  Description of Artificial Sequence Synthetic peptide
ANXTNFTNET

SEQ ID NO: 119 - protein, 9 aa, Artificial Sequence
  Description of Artificial Sequence Synthetic peptide
ANLDKLHKH

SEQ ID NO: 120 - protein, 11 aa, Artificial Sequence
  Description of Artificial Sequence Synthetic peptide
ANCFTNQTNFT

SEQ ID NO: 121 - protein, 11 aa, Artificial Sequence
  Description of Artificial Sequence Synthetic peptide
ANWTNWTNEWT

SEQ ID NO: 122 - protein, 10 aa, Artificial Sequence
  Description of Artificial Sequence Synthetic peptide
ANCTNWTNCT

SEQ ID NO: 123 - protein, 10 aa, Artificial Sequence
  Description of Artificial Sequence Synthetic peptide
CHPYNWTNWT

SEQ ID NO: 124 - protein, 10 aa, Artificial Sequence
  Description of Artificial Sequence Synthetic peptide
ANETNYTNET

SEQ ID NO: 125 - protein, 7 aa, Artificial Sequence
  Description of Artificial Sequence Synthetic peptide
ANWTNWT

SEQ ID NO: 126 - protein, 10 aa, Artificial Sequence
  Description of Artificial Sequence Synthetic peptide
AKPYKSYKFY

SEQ ID NO: 127 - protein, 10 aa, Artificial Sequence
  Description of Artificial Sequence Synthetic peptide
ANITNKTNWT

SEQ ID NO: 128 - protein, 10 aa, Artificial Sequence
  Description of Artificial Sequence Synthetic peptide
ANWTNMTNIT

SEQ ID NO: 129 - protein, 10 aa, Artificial Sequence
  Description of Artificial Sequence Synthetic peptide
ANNTNRTNFT

SEQ ID NO: 130 - protein, 10 aa, Artificial Sequence
  Description of Artificial Sequence Synthetic peptide
ANWTNWTNWT

SEQ ID NO: 131 - protein, 11 aa, Artificial Sequence
  Description of Artificial Sequence Synthetic peptide
ANWRTNHTNKT

SEQ ID NO: 132 - protein, 10 aa, Artificial Sequence
  Description of Artificial Sequence Synthetic peptide
ANQTNITNWT

SEQ ID NO: 133 - protein, 11 aa, Artificial Sequence
  Description of Artificial Sequence Synthetic peptide
ANFTNVATNQT

SEQ ID NO: 134 - protein, 10 aa, Artificial Sequence
  Description of Artificial Sequence Synthetic peptide
ANTTXLTNKT

SEQ ID NO: 135 - protein, 10 aa, Artificial Sequence
  Description of Artificial Sequence Synthetic peptide
ANKTNXTNIT

SEQ ID NO: 136 - protein, 10 aa, Artificial Sequence
  Description of Artificial Sequence Synthetic peptide
ANWTNCTNIT

SEQ ID NO: 137 - protein, 10 aa, Artificial Sequence
  Description of Artificial Sequence Synthetic peptide
ANWTNXTNWT

SEQ ID NO: 138 - protein, 10 aa, Artificial Sequence
  Description of Artificial Sequence Synthetic peptide
CQLDRSTNET

SEQ ID NO: 139 - protein, 10 aa, Artificial Sequence
  Description of Artificial Sequence Synthetic peptide
ANNTNYTNWT

SEQ ID NO: 140 - protein, 10 aa, Artificial Sequence
  Description of Artificial Sequence Synthetic peptide
ANNTNYTNWT

SEQ ID NO: 141 - protein, 12 aa, Artificial Sequence
  Description of Artificial Sequence Synthetic peptide
AANDTNWTVNCT

SEQ ID NO: 142 - protein, 13 aa, Artificial Sequence
  Description of Artificial Sequence Synthetic peptide
ATNITLNYTANTT

SEQ ID NO: 143 - protein, 13 aa, Artificial Sequence
  Description of Artificial Sequence Synthetic peptide
AANSTGNITINGT

SEQ ID NO: 144 - protein, 13 aa, Artificial Sequence
  Description of Artificial Sequence Synthetic peptide
AVNWTSNDTSNST

SEQ ID NO: 145 - protein, 13 aa, Artificial Sequence
  Description of Artificial Sequence Synthetic peptide
ASPINATSPINAT

SEQ ID NO: 146 - protein, 4 aa, Artificial Sequence
  Description of Artificial Sequence Linker
GGGG

SEQ ID NO: 147 - protein, 4 aa, Artificial Sequence
  Description of Artificial Sequence Linker
GNAT